SEQUENCE LISTING (88 sequences)

SEQ ID NO: 1 - DNA, 5730 bp, synthetic construct
  misc_feature 1..5730: note = LAT-derived sequence
  source 1..5730: mol_type = other DNA
cacggcggccaccgccgccgccgccgccgacaccgcagagccggcgcgcgcacacacaagcggcagaggcagaaaggccccgagtcattgtttatgtggccgcgggccagcagacggcccgcgacacccccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccatgtggtgctgtggcctgtttttgctgcgtcatctgagcctttataaaagcgggggcgcggccgtgccgatcgccggtggtgcgaaagactttccgggcgcgtccgggtgccgcggctctccgggcccccctgcagccggggcggccaaggggcgtcggcgacatcctccccctaagcgccggccggccgctggtctgttttttcgttttccccgtttcgggggtggtgggggttgcggtttctgtttctttaacccgtctggggtgtttttcgttccgtcgccggaatgtttcgttcgtctgtcccctcacggggcgaaggccgcgtacggcccgggacgaggggcccccgaccgcggcggtccgggccccgtccggacccgctcgccggcacgcgacgcgaaaaaggccccccggaggcttttccgggttcccggcccggggcctgagatgaacactcggggttaccgccaacggccggcccccgtggcggcccggcccggggccccggcggacccaaggggccccggcccggggccccacaacggcccggcgcatgcgctgtggtttttttttcctcggtgttctgccgggctccgtcgcctttcctgttctcgcttctcccccccccccttcacccccagtaccctcctccctcccttcctcccccgttatcccactcgtcgagggcgccccggtgtcgttcaacaaagacgccgcgtttccaggtaggttagacacctgcttctccccaatagagggggggggacccaaacgacagggggcgccccagaggctaaggtcggccacgccactcgcgggtgggctcgtgttacagcacaccagcccgttattttccccccctcccacccttagttagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggagtgcgggggagggggggggtgtggaaggaaatgcacggcgcgtgtgtaccccccctaaagtttgtcctaaagcgaggatatggaggagtggcgggtgccgggggaccggggtgatctctggcacgcgggggtgggaagggtcgggggagggggggatggggtaccggcccacctggccggcgcgggtgcgcgtgcctttgcacaccaaccccacgtcccccggcggtctctaagaaacaccgccccccctccttcataccaccgagcatgcctgggtgtgggttggtaaccaacacgcccatcccctcgtctcctgtgattctctggctgcaccgcattcttgttttctaactatgttcctgtttctgtctccccccccacccctccgccccaccccccaacacccacgtctgtgggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggaaaaagcgaagcgcgcggcgggcgggagtcgctgcgcgctgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcggctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttaaattgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccgcgatacatccaacacagacagggaaaagatacaaaagtaaacctttatttcccaacagacagcaaaaatcccctgagttttttttattagggccaacacaaaagacccgctggtgtgtggtgcccgtgtctttcacttttcccctccccgacacggattggctggtgtagtgggcgcggccagagaccacccagcgcccgcaattggatatcggggcccgcggtaccgtcgactgcagaattcgaagcttgagctcgagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttctt

SEQ ID NO: 2 - DNA, 20 bp, synthetic construct
  misc_feature 1..20: note = primer
  source 1..20: mol_type = other DNA
gcggcggaaaatagcctttg

SEQ ID NO: 3 - DNA, 21 bp, synthetic construct
  misc_feature 1..21: note = primer
  source 1..21: mol_type = other DNA
gatcacacgttccacctcatc

SEQ ID NO: 4 - DNA, 24 bp, synthetic construct
  misc_feature 1..24: note = primer
  source 1..24: mol_type = other DNA
atcatggccgacaagcagaagaac

SEQ ID NO: 5 - DNA, 24 bp, synthetic construct
  misc_feature 1..24: note = primer
  source 1..24: mol_type = other DNA
gtacagctcgtccatgccgagagt

SEQ ID NO: 6 - DNA, 20 bp, synthetic construct
  misc_feature 1..20: note = primer
  source 1..20: mol_type = other DNA
cctgtcccctcagttcatgt

SEQ ID NO: 7 - DNA, 20 bp, synthetic construct
  misc_feature 1..20: note = primer
  source 1..20: mol_type = other DNA
gcttcaagtagtcggggatg

SEQ ID NO: 8 - DNA, 19 bp, synthetic construct
  misc_feature 1..19: note = primer
  source 1..19: mol_type = other DNA
accaccatgacgacgactc

SEQ ID NO: 9 - DNA, 18 bp, synthetic construct
  misc_feature 1..18: note = primer
  source 1..18: mol_type = other DNA
agccccgtctcgaacagt

SEQ ID NO: 10 - DNA, 19 bp, synthetic construct
  misc_feature 1..19: note = primer
  source 1..19: mol_type = other DNA
cgtgtgcaagcttccttgt

SEQ ID NO: 11 - DNA, 26 bp, synthetic construct
  misc_feature 1..26: note = primer
  source 1..26: mol_type = other DNA
tctcgagattactaagatcacactcc

SEQ ID NO: 12 - DNA, 26 bp, synthetic construct
  misc_feature 1..26: note = primer
  source 1..26: mol_type = other DNA
tctcgagattactaagatcacactcc

SEQ ID NO: 13 - DNA, 23 bp, synthetic construct
  misc_feature 1..23: note = primer
  source 1..23: mol_type = other DNA
aaaacagggagttgcaataaaaa

SEQ ID NO: 14 - DNA, 18 bp, synthetic construct
  misc_feature 1..18: note = primer
  source 1..18: mol_type = other DNA
atggggtggctccagaac

SEQ ID NO: 15 - DNA, 18 bp, synthetic construct
  misc_feature 1..18: note = primer
  source 1..18: mol_type = other DNA
ctgccggtgatgaaggag

SEQ ID NO: 16 - DNA, 19 bp, synthetic construct
  misc_feature 1..19: note = primer
  source 1..19: mol_type = other DNA
gtaacctccacgcccaact

SEQ ID NO: 17 - DNA, 19 bp, synthetic construct
  misc_feature 1..19: note = primer
  source 1..19: mol_type = other DNA
agcagcagcgaacaagaag

SEQ ID NO: 18 - DNA, 18 bp, synthetic construct
  misc_feature 1..18: note = primer
  source 1..18: mol_type = other DNA
atgggcctctcgttctcc

SEQ ID NO: 19 - DNA, 19 bp, synthetic construct
  misc_feature 1..19: note = primer
  source 1..19: mol_type = other DNA
cggtgatgaggacgttgtt

SEQ ID NO: 20 - DNA, 18 bp, synthetic construct
  misc_feature 1..18: note = primer
  source 1..18: mol_type = other DNA
tcatcttcgaccgccatc

SEQ ID NO: 21 - DNA, 19 bp, synthetic construct
  misc_feature 1..19: note = primer
  source 1..19: mol_type = other DNA
tgctgcccataaggtatcg

SEQ ID NO: 22 - DNA, 20 bp, synthetic construct
  misc_feature 1..20: note = primer
  source 1..20: mol_type = other DNA
gtcagcaccttcatcgacct

SEQ ID NO: 23 - DNA, 18 bp, synthetic construct
  misc_feature 1..18: note = primer
  source 1..18: mol_type = other DNA
cagggggacaaactcgtg

SEQ ID NO: 24 - DNA, 19 bp, synthetic construct
  misc_feature 1..19: note = primer
  source 1..19: mol_type = other DNA
agctgcagatcgaggactg

SEQ ID NO: 25 - DNA, 20 bp, synthetic construct
  misc_feature 1..20: note = primer
  source 1..20: mol_type = other DNA
ccatcatctcctcgcttagg

SEQ ID NO: 26 - DNA, 21 bp, synthetic construct
  misc_feature 1..21: note = primer
  source 1..21: mol_type = other DNA
gtctgctctacgacctgtcca

SEQ ID NO: 27 - DNA, 19 bp, synthetic construct
  misc_feature 1..19: note = primer
  source 1..19: mol_type = other DNA
aaccgagcgaaaacaggag

SEQ ID NO: 28 - DNA, 21 bp, synthetic construct
  misc_feature 1..21: note = primer
  source 1..21: mol_type = other DNA
tccatcccaataacacctacg

SEQ ID NO: 29 - DNA, 18 bp, synthetic construct
  misc_feature 1..18: note = primer
  source 1..18: mol_type = other DNA
ggggtccagtgacattcg

SEQ ID NO: 30 - DNA, 18 bp, synthetic construct
  misc_feature 1..18: note = primer
  source 1..18: mol_type = other DNA
gagggtcagccgttcaag

SEQ ID NO: 31 - DNA, 18 bp, synthetic construct
  misc_feature 1..18: note = primer
  source 1..18: mol_type = other DNA
aactccacggggttacgc

SEQ ID NO: 32 - DNA, 19 bp, synthetic construct
  misc_feature 1..19: note = primer
  source 1..19: mol_type = other DNA
gcgctctctcgtttcttcc

SEQ ID NO: 33 - DNA, 18 bp, synthetic construct
  misc_feature 1..18: note = primer
  source 1..18: mol_type = other DNA
ggccaacacggttcgata

SEQ ID NO: 34 - DNA, 20 bp, synthetic construct
  misc_feature 1..20: note = primer
  source 1..20: mol_type = other DNA
caatctgggaagcgtgaatc

SEQ ID NO: 35 - DNA, 18 bp, synthetic construct
  misc_feature 1..18: note = primer
  source 1..18: mol_type = other DNA
cgcccaaagtcaaacgtc

SEQ ID NO: 36 - DNA, 18 bp, synthetic construct
  misc_feature 1..18: note = primer
  source 1..18: mol_type = other DNA
ccacaggaggcctacacg

SEQ ID NO: 37 - DNA, 20 bp, synthetic construct
  misc_feature 1..20: note = primer
  source 1..20: mol_type = other DNA
ctgaaacttctcggggatca

SEQ ID NO: 38 - DNA, 22 bp, synthetic construct
  misc_feature 1..22: note = primer
  source 1..22: mol_type = other DNA
ccccgctggaactactatgaca

SEQ ID NO: 39 - DNA, 19 bp, synthetic construct
  misc_feature 1..19: note = primer
  source 1..19: mol_type = other DNA
gcatcaggaaccccaggtt

SEQ ID NO: 40 - DNA, 20 bp, synthetic construct
  misc_feature 1..20: note = probe has a 5' "FAM" and a 3' "TAMRA"
  source 1..20: mol_type = other DNA
ttcagcgccgtcagcgagga

SEQ ID NO: 41 - DNA, 90 bp, synthetic construct
  misc_feature 1..90: note = primer
  source 1..90: mol_type = other DNA
cccgatatccaattgcgggcgctgggtggtctctggccgcgcccactacaccagccaatccgtgtaggatgacgacgataagtagggata

SEQ ID NO: 42 - DNA, 67 bp, synthetic construct
  misc_feature 1..67: note = primer
  source 1..67: mol_type = other DNA
gatctcgagctcaagcttcgaattctgcagtcgacggtaccgcgggccccgatatccaattgcgggc

SEQ ID NO: 43 - DNA, 75 bp, synthetic construct
  misc_feature 1..75: note = primer
  source 1..75: mol_type = other DNA
acacggattggctggtgtagtgggcgcggccagagaccacccagcgcccgcaaccaattaaccaattctgattag

SEQ ID NO: 44 - DNA, 84 bp, synthetic construct
  misc_feature 1..84: note = primer
  source 1..84: mol_type = other DNA
tatggatcccggacctggttaaccacccgccggtcctacgcgaactggaggataagcgcaggatgacgacgataagtagggata

SEQ ID NO: 45 - DNA, 84 bp, synthetic construct
  misc_feature 1..84: note = primer
  source 1..84: mol_type = other DNA
caggaattcgcgcttatcctccagttcgcgtaggaccggcgggtggttaaccaggtccgcaaccaattaaccaattctgattag

SEQ ID NO: 46 - DNA, 81 bp, synthetic construct
  misc_feature 1..81: note = primer
  source 1..81: mol_type = other DNA
gggccctggaaatggcggacaccttcctggacaccatgcgggttgggcccaggatgacgacgataagtagggataacaggg

SEQ ID NO: 47 - DNA, 75 bp, synthetic construct
  misc_feature 1..75: note = primer
  source 1..75: mol_type = other DNA
gggcccaacccgcatggtgtccaggaaggtgtccgccatttccagggccccaaccaattaaccaattctgattag

SEQ ID NO: 48 - DNA, 73 bp, synthetic construct
  misc_feature 1..73: note = primer
  source 1..73: mol_type = other DNA
tttataaccccgggggtcattcccaacgatcacatgcaatctaactggctgggccctggaaatggcggacacc

SEQ ID NO: 49 - DNA, 75 bp, synthetic construct
  misc_feature 1..75: note = primer
  source 1..75: mol_type = other DNA
tatggatccccgcggatggtgagcaagggcgaggaggataacatggccatcataggatgacgacgataagtaggg

SEQ ID NO: 50 - DNA, 43 bp, synthetic construct
  misc_feature 1..43: note = primer
  source 1..43: mol_type = other DNA
aatctgcagggatccctacaaccaattaaccaattctgattag

SEQ ID NO: 51 - DNA, 75 bp, synthetic construct
  misc_feature 1..75: note = primer
  source 1..75: mol_type = other DNA
cttggggcggtcccgcccgccggccaatgggggggcggcaaggcgggcggtggcggccgctctagaagatctggc

SEQ ID NO: 52 - DNA, 75 bp, synthetic construct
  misc_feature 1..75: note = primer
  source 1..75: mol_type = other DNA
ccgcggggggcccgggctgccacaggtgaaaccaacagagcacggcgcacgctgggtaccgggccccccctcgag

SEQ ID NO: 53 - DNA, 75 bp, synthetic construct
  misc_feature 1..75: note = primer
  source 1..75: mol_type = other DNA
cctcactgcccgtcgcgcgtgtttgatgttaataaataacacataaattttggcggccgctctagaagatctggc

SEQ ID NO: 54 - DNA, 75 bp, synthetic construct
  misc_feature 1..75: note = primer
  source 1..75: mol_type = other DNA
ccgacactgaaatgcccccccccccttgcgggcggtccattaaagacaacgctgggtaccgggccccccctcgag

SEQ ID NO: 55 - DNA, 81 bp, synthetic construct
  misc_feature 1..81: note = primer
  source 1..81: mol_type = other DNA
tattacgtattagtcatcgctattaccatggtcgaggtgagccccacgttctgcttaggatgacgacgataagtagggata

SEQ ID NO: 56 - DNA, 43 bp, synthetic construct
  misc_feature 1..43: note = primer
  source 1..43: mol_type = other DNA
aatctgcagtacgtactacaaccaattaaccaattctgattag

SEQ ID NO: 57 - DNA, 36 bp, synthetic construct
  misc_feature 1..36: note = primer
  source 1..36: mol_type = other DNA
gcaattggctctgccccgccgtccccgtgttcgtcc

SEQ ID NO: 58 - DNA, 75 bp, synthetic construct
  misc_feature 1..75: note = primer
  source 1..75: mol_type = other DNA
tttttgcaaaagcctaggcctccaataactagtcaataatcaatgtcgacttatttattaacatcaaacacgcgc

SEQ ID NO: 59 - DNA, 36 bp, synthetic construct
  misc_feature 1..36: note = primer
  source 1..36: mol_type = other DNA
gcacgcgtagaggtgctgcgggagattcaactgagc

SEQ ID NO: 60 - DNA, 75 bp, synthetic construct
  misc_feature 1..75: note = primer
  source 1..75: mol_type = other DNA
tattgactagttattggaggcctaggcttttgcaaaaaagcttataatgggtctttaatggaccgcccgcaaggg

SEQ ID NO: 61 - DNA, 75 bp, synthetic construct
  misc_feature 1..75: note = primer
  source 1..75: mol_type = other DNA
tattacgtacaaccacatacagcgccatgtcaacgatatgttgggccgcgttgaggatgacgacgataagtaggg

SEQ ID NO: 62 - DNA, 43 bp, synthetic construct; identical to SEQ ID NO: 56
  misc_feature 1..43: note = primer
  source 1..43: mol_type = other DNA

SEQ ID NO: 63 - DNA, 80 bp, synthetic construct
  misc_feature 1..80: note = primer
  source 1..80: mol_type = other DNA
tatacccgtgacacccgacgctggggggcgtggctgccgggaggggccgcgtatgaggatgacgacgataagtagggata

SEQ ID NO: 64 - DNA, 70 bp, synthetic construct
  misc_feature 1..70: note = primer
  source 1..70: mol_type = other DNA
ccacacaagccccgtatccccgttcccgcgcttttcgttggtttatatacccgtgacacccgacgctggg

SEQ ID NO: 65 - DNA, 75 bp, synthetic construct
  misc_feature 1..75: note = primer
  source 1..75: mol_type = other DNA
catacgcggcccctcccggcagccacgccccccagcgtcgggtgtcacggcaaccaattaaccaattctgattag

SEQ ID NO: 66 - DNA, 75 bp, synthetic construct; identical to SEQ ID NO: 65
  misc_feature 1..75: note = primer
  source 1..75: mol_type = other DNA

SEQ ID NO: 67 - DNA, 70 bp, synthetic construct
  misc_feature 1..70: note = primer
  source 1..70: mol_type = other DNA
gtcaggcagcccgggccgcggctctgtggttaacaccagagcctgcccaatccaacacagacagggaaaa

SEQ ID NO: 68 - DNA, 75 bp, synthetic construct
  misc_feature 1..75: note = primer
  source 1..75: mol_type = other DNA
tgttgggaaataaaggtttacttttgtatcttttccctgtctgtgttggacaaccaattaaccaattctgattag

SEQ ID NO: 69 - DNA, 80 bp, synthetic construct
  misc_feature 1..80: note = primer
  source 1..80: mol_type = other DNA
cagatagtaatcaattacggggtcattagttcatagcccatatatggagttccaggatgacgacgataagtagggataac

SEQ ID NO: 70 - DNA, 70 bp, synthetic construct
  misc_feature 1..70: note = primer
  source 1..70: mol_type = other DNA
agactttccgggcgcgtccgggtgccgcggctctccgggcccccctgcagatagtaatcaattacggggt

SEQ ID NO: 71 - DNA, 75 bp, synthetic construct
  misc_feature 1..75: note = primer
  source 1..75: mol_type = other DNA
ggaactccatatatgggctatgaactaatgaccccgtaattgattactatcaaccaattaaccaattctgattag

SEQ ID NO: 72 - DNA, 43 bp, synthetic construct
  misc_feature 1..43: note = primer
  source 1..43: mol_type = other DNA
tgacaattgactagttatacaagtttgtacaaaaaagctgaac

SEQ ID NO: 73 - DNA, 46 bp, synthetic construct
  misc_feature 1..46: note = primer
  source 1..46: mol_type = other DNA
ccattataacaattgagatctccactttgtacaagaaagctgaacg

SEQ ID NO: 74 - DNA, 75 bp, synthetic construct
  misc_feature 1..75: note = primer
  source 1..75: mol_type = other DNA
cggacccaaaataataaacacacaatcacgtgcgataaaaagaacacgcgacaagtttgtacaaaaaagctgaac

SEQ ID NO: 75 - DNA, 75 bp, synthetic construct
  misc_feature 1..75: note = primer
  source 1..75: mol_type = other DNA
cctgtttgtcgacgagatttaataaaaataaccaaaaacaccacaggggaccactttgtacaagaaagctgaacg

SEQ ID NO: 76 - DNA, 75 bp, synthetic construct
  misc_feature 1..75: note = primer
  source 1..75: mol_type = other DNA
aaacgtttgtatctcatctttcctgtgtgtagttgtttctgttggatgccatatcacaagtttgtacaaaaaagc

SEQ ID NO: 77 - DNA, 75 bp, synthetic construct
  misc_feature 1..75: note = primer
  source 1..75: mol_type = other DNA
tgcgtgttttcatccaacccgtgtgttctgtgtttgtgggatggaggggccgagaaacgtaaaatgatataaata

SEQ ID NO: 78 - DNA, 75 bp, synthetic construct
  misc_feature 1..75: note = primer
  source 1..75: mol_type = other DNA
ccggggacaccgccagcaaacgcgagcaacgggccacggggatgaagcagaggatgacgacgataagtagggata

SEQ ID NO: 79 - DNA, 75 bp, synthetic construct
  misc_feature 1..75: note = primer
  source 1..75: mol_type = other DNA
ccacacaagccccgtatccccgttcccgcgcttttcgttggtttatatacccggggacaccgccagcaaacgcga

SEQ ID NO: 80 - DNA, 75 bp, synthetic construct
  misc_feature 1..75: note = primer
  source 1..75: mol_type = other DNA
ctgcttcatccccgtggcccgttgctcgcgtttgctggcggtgtccccggcaaccaattaaccaattctgattag

SEQ ID NO: 81 - DNA, 31 bp, synthetic construct
  misc_feature 1..31: note = primer
  source 1..31: mol_type = other DNA
cccggccaaccagcgtccgccgagtcttcgg

SEQ ID NO: 82 - DNA, 30 bp, synthetic construct
  misc_feature 1..30: note = primer
  source 1..30: mol_type = other DNA
gccggttccagtgtaagggtcgggggtccc

SEQ ID NO: 83 - DNA, 20 bp, synthetic construct
  misc_feature 1..20: note = primer
  source 1..20: mol_type = other DNA
cctgacagagctgtattgta

SEQ ID NO: 84 - DNA, 21 bp, synthetic construct
  misc_feature 1..21: note = primer
  source 1..21: mol_type = other DNA
agatcgctgtcggagaggtcc

SEQ ID NO: 85 - DNA, 75 bp, synthetic construct
  misc_feature 1..75: note = primer
  source 1..75: mol_type = other DNA
tatagatctcgagctcaagcttcgaattctgcagtcgacggtaccgcgggcccaggatgacgacgataagtaggg

SEQ ID NO: 86 - DNA, 43 bp, synthetic construct
  misc_feature 1..43: note = primer
  source 1..43: mol_type = other DNA
aatctgcagagatctctacaaccaattaaccaattctgattag

SEQ ID NO: 87 - DNA, 75 bp, synthetic construct
  misc_feature 1..75: note = primer
  source 1..75: mol_type = other DNA
tatgttaacaacaacaattgcattcattttatgtttcaggttcagggggaggtaggatgacgacgataagtaggg

SEQ ID NO: 88 - DNA, 43 bp, synthetic construct
  misc_feature 1..43: note = primer
  source 1..43: mol_type = other DNA
aatctgcaggttaacctacaaccaattaaccaattctgattag

(19) United States
(12) Patent Application Publication
(10) Pub. No.: US 2025/0109411 A1
(43) Pub. Date: Apr. 3, 2025

(54) NON-TOXIC HSV VECTORS FOR EFFICIENT GENE DELIVERY APPLICATIONS AND COMPLEMENTING CELLS FOR THEIR PRODUCTION

(71) Applicant: University of Pittsburgh - of the Commonwealth System of Higher Education, Pittsburgh, PA (US)

(72) Inventors: Joseph C. Glorioso, III, Pittsburgh, PA (US); Justus Cohen, Allison Park, PA (US); Yoshitaka Miyagawa, Pittsburgh, PA (US); David Krisky, Sewickley, PA (US); James Wechuck, Glenshaw, PA (US); Darren Wolfe, Pittsburgh, PA (US)

(73) Assignee: University of Pittsburgh - of the Commonwealth System of Higher Education, Pittsburgh, PA (US)

(21) Appl. No.: 18/921,539

(22) Filed: Oct. 21, 2024

Related U.S. Application Data
(63) Continuation of application No. 17/703,762, filed on Mar. 24, 2022, now abandoned.
Application No. 17/703,762 is a continuation of application No. 16/199,715, filed on Nov. 26, 2018, now abandoned.
Application No. 16/199,715 is a continuation of application No. 14/905,708, filed on Jan. 15, 2016, now Pat. No. 10,174,341.
(60) Provisional application No. 61/847,405, filed on Jul. 17, 2013.

(51) Int. Cl. C12N 15/86; A61K 35/763; A61K 48/00; C12N 7/00

(52) U.S. Cl. CPC C12N 15/86; A61K 35/763; A61K 48/0066; C12N 7/00; C12N 2710/16621; C12N 2710/16643; C12N 2710/16671

(57) ABSTRACT

Disclosed is a method for administering a transgene into a fibroblast in a subject comprising: a) providing a herpes simplex virus (HSV) comprising a recombinant herpes simplex virus genome, wherein said recombinant herpes simplex virus genome comprises one or more transgenes encoding a polypeptide to be expressed in said fibroblast; and b) providing a pharmaceutically acceptable carrier, wherein said HSV has reduced cytotoxicity as compared to a wild-type herpes simplex virus.

### CROSS-REFERENCE TO RELATED APPLICATIONS

This application is a continuation of U.S. patent application Ser. No. 17/703,762, filed Mar. 24, 2022, which is a continuation of Ser. No. 16/199,715, filed Nov. 26, 2018, now abandoned, which is a continuation of U.S. patent application Ser. No. 14/905,708, filed Jan. 15, 2016, now U.S. Pat. No. 10,174,341, which is the U.S. National Phase of International Patent Application No. PCT/US2014/047068, filed Jul. 17, 2014, which claims the benefit of U.S. Provisional Patent Application No. 61/847,405, filed Jul. 17, 2013, each of which is incorporated by reference in its entirety herein.

### STATEMENT REGARDING FEDERALLY SPONSORED RESEARCH AND DEVELOPMENT

This invention was made with Government support under Grant Numbers PO1DK044935 and 5RO1NS064988 awarded by the National Institutes of Health. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 119,247 Byte XML file named “772030_ST26.XML,” dated Oct. 21, 2024.

### BACKGROUND OF THE INVENTION

Among many viral and non-viral genetic vector systems, Herpes Simplex Virus (HSV)-based vectors have been investigated for use as gene transfer vectors, including for possible therapeutic use in human patients. HSV is a complex, non-integrating DNA virus capable of infecting a very wide range of human and animal cells. The viral genome contains more than 80 genes and is composed of two unique segments, UL and Us, each flanked by inverted repeats that encode critical diploid genes. An important feature of HSV replication is the expression of its genes in waves referred to as cascade regulation (Rajcani, Virus Genes, 28:293-310 (2004)). Removal of the essential immediate-early (IE) genes ICP27 and ICP4 renders the virus completely defective and incapable of expression of early (E) genes involved in viral genome replication and late (L) genes functioning in progeny virion assembly. These replication-defective viruses can be grown on complementing cells that express (complement) the missing ICP4 and ICP27 gene products and can then be used to infect non-complementing cells where the viral genome takes residence as a stable nuclear episome. However, vectors that preserve the IC0 and ICP22 IE genes are toxic to cells, but inactivation or deletion of these genes, the ICP0 gene in particular, hampers transgene expression.

Accordingly, there remains a need for an HSV vector capable of expressing a transgene in any tissue or cell, in vitro or in vivo, without harming the cell or tissue and a system for propagating such vectors.

### BRIEF SUMMARY OF THE INVENTION

The present invention relates to a breakthrough in HSV vector engineering that has provided the opportunity to express transgenes in a wide variety of tissues or cells (particularly mammalian), in vitro or in vivo, without expression of any harmful viral genes. The inventive HSV vector does not express any toxic viral genes in non-complementing cells, yet is capable of vigorous, persistent (e.g., for at least 14 days, such as at least 28 days, and preferably for at least 60 days) transgene expression. As such, it fills an extremely important niche in vector technology since HSV is the only vector that combines the capability of carrying large single-or multi-transgene expression cassettes controlled by general or cell-specific promoters with the property of highly efficient infection without vector integration. The inventive vector will allow efficient gene delivery to tissues, such as liver, for which no effective vector is currently available.

In one embodiment, the invention provides a herpes simplex virus (HSV) vector that does not express toxic HSV genes in non-complementing cells and which comprises a genome comprising one or more transgenes, wherein the vector is capable of expression of a transgene for at least 28 days in non-complementing cells. The inventive vctor can comprise a transgene inserted in operable connection with one or more insulator sequences within the genome, wherein the vector does not express ICP0, ICP4, ICP22, ICP27, and ICP47 as immediate early genes. Depending on the activity of the promoter controlling the transgene, the inventive vector can express the transgene in any type of mammalian (especially human) cell that it can infect without the cytotoxicity associated with viral gene expression.

In another aspect, the invention provides a complementing cell for the production of the inventive vector. The inventive cell line is derived from U2OS cells, which have been engineered to express ICP4 and ICP27 when the cell is infected with HSV.

### BRIEF DESCRIPTION OF THE DRAWINGS

FIG. 1A is a vector schematic showing reporter gene expression. The top line represents the full-length wild-type HSV-1 strain KOS BAC clone (Gierash, J. Virol. Meth., 135: 197-206 (2006)) used to generate the JΔNI vectors of the invention. The second line represents JΔNI5, i.e. the backbone of JΔNI7-GFP. The third line is a blow-up of the LAT to UL4 region of JΔNI5. The left side of the 4th line (LAT:CAG-GFP) shows the position of the reporter expression cassette in JΔNI7-GFP. The right side of the 4th line (UL3/4:CAG-GFP) shows the position of the same expression cassette in a JΔNI5-derived control vector (JΔNI6-CAGGFP). FIG. 1A key: TR, terminal repeats: IR, internal repeats: UL, unique long region: US, unique short: numbers identify the location of the different IE genes: Δ, deletion; β, early promoter: CTRL, insulator: LAT P2, long-term expression element; CAG, CMV/actin/globin enhancer/promoter/intron cassette. FIG. 1B is a set of photographs showing reporter gene expression. FIG. 1B key: HDF, human dermal fibroblasts: hpi, hours post-infection: dpi, days post-infection: MOI, multiplicity of infection.

FIGS. 2A-2B is a set of photographs comparing transgene expression in JΔNI6-CAGGFP- and JΔANI7-GFP-infected cells. FIG. 2A shows GFP (“EGFP”) and mCherry expression in HDF and U2OS cells infected at the indicated MOIs. Fluorescence microscopy images were acquired 3 days after infection. FIG. 2B shows the duration of transgene expression in infected HDF (MOI-0.5). Images were acquired at 1-14 days post-infection.

FIG. 3 is a set of photographs showing expansion of JΔNI7-miR302GFP virus on complementing (U2OS-ICP4/ICP27) cells. Photographs were taken at 3 days post-infection.

FIG. 4 is a schematic of the construction of a targeting plasmid for insertion of a tetracycline-inducible promoter and Gateway recombination cassette into the LAT locus of a HSV vector. FIG. 4 key: Zeo, zeomycin-resistance gene: Cm, chloramphenicol-resistance gene: ccdB, toxin gene for negative selection: LATP2, LAT long-term expression element: CTRL2, chromatin boundary/insulator element 2 of the LAT locus.

FIG. 5 is a schematic of the construction of a lentivirus plasmid for expressing ICP27 in a U2OS-ICP4 cell line. FIG. 5 key: p, promoter: bla, blasticidin resistance gene.

FIG. 6A is a set of photographs showing ICP27 immunofluorescence staining of U2OS-ICP4 cells and different clonal U2OS-ICP4/ICP27 cells infected with QOZHG virus (ICP4-null: ICP27-null). FIG. 6B is a set of photographs showing the growth of the QOZHG virus (GFP expression from an HCMV promoter-GFP cassette in the viral genome) in U2OS-ICP4 and U2OS-ICP4/ICP27 cells.

FIG. 7 is a schematic of a HSV vector of the present invention (JΔNI7-GFP, center), the LAT region of JΔNI7-GFP (top), and the LAT region of a wild-type HSV (bottom).

FIGS. 8A-8E show the sequence of the LAT region of a JΔNI7-GFP HSV vector of the present invention. This sequence is SEQ ID NO: 1.

FIGS. 9A-9C. Vector genome structures and complementing cells for virus production. (FIG. 9A) Schematic representations of the wild-type HSV-1 KOS genome in KOS-37 BAC (24) and the genomes of the IE gene-deleted derivatives JΔNI2, JΔANI3 and JΔNI5. UL, unique long segment: US, unique short segment. Open boxes: terminal and internal inverted repeats. The BAC elements, including a chloramphenicol-resistance gene and β-galactosidase expression cassette, are located between loxP sites in the UL37-UL38 intergenic region (Gierasch et al., J. Virol. Methods 135, 197-206 (2006)). The US region in KOS-37 BAC and its derivatives is inverted compared to the standard representation of the HSV genome. Deletions in the JΔNI constructs are indicated by black boxes and the Δ symbol: the ICP47 promoter and translation initiation codon are removed as part of the joint deletion. IE genes converted to early expression kinetics by promoter replacement (ICP0, ICP27) or TAATGARAT deletion (ICP22) are represented by hatched boxes and the β symbol in front of the ICP number. All JΔNI recombinants contain the hyper-activating N/T mutations in the gB gene (Uchida et al., J. Virol. 84, 12200-09 (2010)) and a ubiquitin C promoter (UbCp)-mCherry cassette in the ICP4 locus: the SV40 poly A region of the mCherry cassette is represented by a small patterned box. (FIG. 9B) Western blot analysis of complementing cells. Uninfected cells and cells infected with QOZHG (left) or JΔNI5 virus (right) at an MOI of 1 were harvested at 24 hpi and extracts were prepared for gel electrophoresis. Blots were probed with antibodies for ICP4, ICP27, or a-tubulin as a loading control. (FIG. 9C) JΔNI2 and JΔNI5 virus growth in U2OS, U2OS-ICP4, U2OS-ICP4/27, and Vero-7b cells. Cells were infected at an MOI of 0.001 and extracellular virus was harvested daily from triplicate wells and titered on U2OS-ICP4/27 cells.

FIGS. 10A-10B. Relative nuclear viral DNA levels after infection with equal gc or PFU. HDFs were infected with the indicated JΔNI vectors at 5,000 gc/cell (FIG. 10A) or 1 PFU/cell (FIG. 10B). At 2 hpi, nuclear DNA was isolated and relative viral gc numbers were determined by qPCR for the gD gene normalized to the cellular 18S rRNA genes.

FIGS. 11A-11D. JΔANI cytotoxicity and viral gene expression in non-complementing cells. (FIG. 11A) In vitro cytotoxicity assay. HDFs and Vero cells were infected at 25,000 gc/cell and cell viability in triplicate wells was measured at 5 dpi by MTT assay. Plotted values represent the mean ratio of virus-infected to mock-infected cells. Brackets with asterisks indicate statistically significant differences (p<0.05) between JΔNI2- and JΔNI3-infected Vero cells and between JΔNI3- and JΔNI5-infected Vero cells. (FIG. 11B). Immunoblot analysis of IE gene products in HDFs. Cells were infected with KOS, QOZHG or JΔNI viruses at 1 PFU/cell and extracts were prepared at 24 hpi. Blots were probed with antibodies for the indicated IE gene products or α-tubulin as a loading control. (FIG. 11C) JΔNI IE gene expression measured by qRT-PCR. HDFs were infected with the indicated viruses at 1,000 gc/cell. mRNA was isolated at 12 hpi and reverse-transcribed for qPCR determination of the cDNA levels for the genes listed at the top. Expression was normalized to 18S rRNA levels and is shown relative to JΔNI2-infected cells. (FIG. 11D) qRT-PCR analysis for expression of early (upper panels) and late genes (lower panels). HDFs were infected and processed as in (FIG. 11C). ICP6 may be considered a delayed IE gene and is grouped with the early genes here as its expression is reportedly more dependent on ICP0 than on VP16 or ICP4 (Desai et al., J. Virol. 67, 6125-35 (1993): Sze et al., Virus Res. 26, 141-52 (1992): Harkness et al., J. Virol. 88 (12) 6847-61 (2014)).

FIGS. 12A-12D. Reporter gene expression in JΔNI-infected HDFs. (FIG. 12A) mCherry fluorescence. Cells were infected at the indicated gc/cell and photographed at 24 hpi. (FIG. 12B) Relative mCherry mRNA levels. HDFs were infected at 5,000 gc/cell and harvested at 6 hpi for mRNA isolation, reverse transcription and qPCR as in FIG. 3C. (FIG. 12C) mCherry fluorescence in U2OS cells. Cells were infected at 1,000 gc/cell and photographed at 24 hpi. (FIG. 12D) Induction of mCherry expression in JΔNI5-infected HDFs. Cells were infected at the indicted gc/cell, super-infected at 24 h with QOZHG at 5,000 gc/cell, and photographed 24 h later.

FIGS. 13A-13D. JΔNI6GFP and JΔNI7GFP genome structures and reporter gene expression. (FIG. 13A) JΔNI7GFP contains a CAG promoter-EGFP expression cassette in the 2-kb LAT intron region between the LATP2 long-term expression/enhancer region and a downstream CTCF-binding motif (CTRL2) in the intron. LATP2 extends from the LAT transcription initiation site to within the 2-kb intron. JΔNI6GFP contains the same CAG promoter-EGFP expression cassette between the UL3 and UL4 genes. The rabbit β-globin poly A region of the CAGp-EGFP cassette is represented by a small patterned box. (B-D) EGFP and mCherry expression in infected HDFs. (FIG. 13B) Cells were infected with JΔNI6GFP or JΔNI7GFP virus at different gc/cell and fluorescence was visualized at 3 dpi. (FIG. 13C) HDFs were infected with JΔNI6GFP or JΔNI7GFP vector at 12,500 gc/cell and harvested 3 or 5 d later for mRNA extraction and qRT-PCR analysis for the 2 reporter genes. Expression normalized to 18S rRNA is shown relative to JΔNI6GFP-infected cells on day 3. (FIG. 13D) HDFs were infected with JΔNI6GFP or JΔNI7GFP virus at 25,000 gc/cell and EGFP fluorescence was photographed at 7, 14 and 28 dpi.

FIGS. 14A-14C. The effect of LAT locus elements on EGFP expression from JΔNI7GFP. (FIG. 14A) Genome representations of JΔNI7GFP and derivatives deleted for CTRL1 (ΔC1), CTRL2 (JΔC2) or LATP2 (ΔLP2) individually or in combinations. The deletion of positions 8978-9161 in JQ673480 encompassed CTRL1, and a deletion of positions 5694-5857 in JQ673480 encompassed CTRL2. These deletions encompass a few bases beyond the CTCF binding motifs. (FIG. 14B) Reporter gene expressin in infected HDFs. Cells were infected with the indicated viruses at 12,500 gc/cell and fluorescence was recorded at 3 dpi. (FIG. 14C) Relative EGFP mRNA levels in HDFs infected with JΔNI7GFP, derivatives deleted for LAT elements, or JΔNI6GFP: viruses are identified by abbreviated names. Cells were infected at 12,500 gc/cell and processed at 3 dpi for qRT-PCR analysis. Expression levels were normalized to 18S rRNA and are presented relative to the level in JΔNI7GFP-infected cells.

FIGS. 15A-15D. Anti-silencing activity of LAT sequences positioned elsewhere in the viral genome. (FIG. 15A) Construction of JΔNI9 and JΔNI10 vectors. An Xhol fragment encompassing CTRL1, LATP2 and CTRL2 was removed from the JΔNI5 genome and a GW recombination cassette was introduced between UL45 and UL46 to generate JΔNI9GW or between UL50 and UL51 to produce JΔANI10GW (upper). The same XhoI sites were used to isolate a CAGp-GFP-containing LAT fragment from JΔNI7GFP (lower left). The XhoI fragment was cloned into pENTRIA (lower right) and transferred into JΔNI9GW or JΔNI10GW by attL/attR recombination with the respective GW cassettes (LR reaction) to produce JΔNI9LAT-GFP and JΔNI10LAT-GFP, respectively. As controls, the CAGp-GFP cassette without LAT sequences was recombined via a pENTRIA intermediate into the GW locus of JΔNI9GW or JΔNI1OGW, producing JΔNI9GFP and JΔNI10GFP. (FIG. 15B) Reporter gene expression in HDFs infected with JΔNI9 or JΔNI10 viruses. HDFs were infected with the indicated viruses at 12,500 gc/cell. EGFP and mCherry fluorescence were recorded at 3 dpi. (FIG. 15C) EGFP mRNA levels in infected HDFs determined by qRT-PCR as in earlier figures. Levels are shown relative to JΔNI9GFP-or JΔNI10GFP-infected cells. JΔANI10ΔC12LP2-GFP was constructed by transfer of the XhoI LAT fragment from JΔNI7AC12LP2-GFP into the GW site of JΔNI10GW similar to the construction of JΔNI10LAT-GFP above. (FIG. 15D) Effect of the deletion of both CTRLs and LATP2 from JΔNI10LAT-GFP on transgene expression. HDFs were infected at 12,500 gc/cell and EGFP and mCherry fluorescence were recorded at 3 dpi.

FIGS. 16A-16D. Reporter gene expression from JΔNI vectors in other non-complementing cells. (FIG. 16A) The cells listed at the top were infected with JΔNI6GFP or JΔNI7GFP at the gc/cell indicated below the panels. EGFP and mCherry fluorescence were recorded at 3 dpi. (FIG. 16B) EGFP gene expression in cells infected as in (FIG. 16A) was measured at 3 dpi by qRT-PCR analysis. Results normalized to 18S rRNA are shown relative to JΔNI6GFP-infected cells. (FIG. 16C) hMDSCs were infected with JΔNI6GFP or JΔNI7GFP virus at 50,000 gc/cell and EGFP fluorescence was photographed at 14 and 28 dpi. (FIG. 16D) qRT-PCR determination of EGFP mRNA levels in hEK, hPAD and hHEP cells 3 d after infection with JΔNI10GFP or JΔNI10: LAT-GFP at 12,500 gc/cell. Normalized expression is shown relative JΔNI10 GFP-infected cells.

FIG. 17 graphically depicts the construction of JΔNI8 from JΔNI5.

FIG. 18 presents data concerning the growth of JΔNI8 relative to JΔNI5 in complementing cells (U2OS-ICP4/27) after infection at 1 genome copy (gc)/cell. The upper panel depicts reporter gene expression (mCherry), whereas the lower left panel reports virus yields in plaque forming units (PFU), and the lower right panel reports virus yields in genome copies (gc).

FIG. 19 depicts data demonstrating that viral genes are expressed earlier from JΔNI8 than from JΔNI5 in complementing cells. In each panel, the lower line represents data for JΔNI5, whereas the upper line represents data for JΔAN18. Data were collected by qRT-PCR and is expressed as the fold difference relative to the JΔNI5 data point at 6 hours post-infection (hpi).

FIG. 20 graphically depicts the genomic structure of JDNI7GFP (also referred to as JΔNI7GFP) and JDNI8GFP (also referred to as JΔNI8GFP).

FIG. 21 presents data demonstrating that infection of human dermal fibroblast (HDF) cells with equal amounts of JΔNI8 and JΔNI5 (expressed as gc) results in equal amounts of viral DNA in the nucleus. Data are representative of two hours post infection (hpi).

FIG. 22 presents data comparing the cell viability (MTT) of HDF infected with various HSV vectors at two M.O.I. The lower-X-line in each panel represents the results for JΔNI5, while the upper-X-line in each graph represents the results for JΔNI8.

FIG. 23 presents data comparing the cell viability analysis (MTT) of HDF infected with various HSV vectors at different numbers of viral genomic copies per cell. For the left panel (gc 12500/cell), the M.O.I. of JΔNI5 was 5 PFU/cell, whereas the M.O.I. for JΔNI8 was 18 PFU/cell. For the right panel (gc 25000), the M.O.I. of JΔNI5 was 11, whereas the M.O.I. for JΔNI8 was 33. The lower-X-line in each panel represents the results for JΔNI5, while the upper-X-line in each graph represents the results for JΔNI8.

FIG. 24 presents data comparing the viability of cells infected with KOS, JΔNI5, and JΔNI8 in six cell types (HDF, human neonatal keratinocytes, human neural stem cells, Vero, human preadipocytes, and human hepatocytes). The MTT assay was conducted using 25,000 gc/cell, with data reported at 5 days post infection (dpi).

FIG. 25 presents dose-response data at three days post infection comparing reporter gene expression (mCherry or Enhanced Green Fluorescent Protein (EGFP)) between human dermal fibroblasts (HDF) infected at the indicated gc/cell with either JΔNI7GFP and JΔNI8GFP.

FIG. 26 presents time-course data comparing reporter gene expression (mCherry or EGFP) between human dermal fibroblasts (HDF) infected at 25,000 gc/cell with either JΔNI7GFP and JΔNI8GFP.

FIG. 27 presents the results of experiments comparing reporter gene expression (mCherry or EGFP) for human neonatal keratinocytes three days post infection with either 12,500 gc/cell or 25,000 gc/cell of either JΔI7GFP or JΔNI8GFP.

FIG. 28 presents the results of experiments comparing reporter gene expression (mCherry or EGFP) for rat dorsal root ganglion (DRG) neurons three days post infection with 6250 gc/cell of either JΔNI7GFP or JΔNI8GFP.

FIG. 29 presents the results of experiments investigating transgene expression (mCherry or EGFP) in neural cells infected with JΔNI7GFP. Top, separate (left: 40×) or merged (center, right: 20×) images of EGFP and mCherry fluorescence at the indicated days post infection (dpi: bottom, separate and merged images at 15 dpi (10×).

FIG. 30 is a schematic diagram of pCX4Hyg-Cre.

FIG. 31 is a schematic representation of the generation of pCX4Hyg-Cre.

FIG. 32 presents data comparing EGFP mRNA levels between JΔNI7GFP-and JΔNI6GFP-infected HDF cells as determined by quantitative reverse transcription (RT)-PCR.

### DETAILED DESCRIPTION OF THE INVENTION

The following patents and publications, which relate to various HSV vector technologies, are herein incorporated by reference. U.S. Pat. No. 5,658,724 relates to HSV strains deficient for ICP4 and ICP27 and methods for their production, growth, and use. U.S. Pat. No. 5,804,413 relates to a cell line comprising DNA encoding ICP4, ICP27, and ICP0. U.S. Pat. No. 5,849,571 relates to latency active herpes virus promoters and their use. U.S. Pat. No. 5,849,572 relates to a HSV-1 vector containing a LAT promoter. U.S. Pat. No. 5,879,934 relates to a recombinant HSV vector comprising genomic mutations within the ICP4 and ICP27 genes such that the ICP4 and ICP27 gene products are defective. U.S. Pat. No. 5,998,174 relates to methods of preparing a HSV vector. U.S. Pat. No. 6,261,552 relates to a HSV vector comprising a HSV genome having a deletion or mutation within a native TAATGARAT sequence, whereby said deletion or mutation causes the kinetics of expression of a native immediate early gene within said genome to be delayed when said genome is within a cell that contains HSV ICP4 gene products. U.S. Pat. No. 7,078,029 relates to HSV having a genome with a mutation of a TAATGARAT sequence such that, in the presence of a ICP4 gene product, a native immediate early gene is expressed from the genome with delayed kinetics. U.S. Pat. No. 7,531,167 relates to a HSV vector comprising deletions in only the ICP4, ICP27, and UL55 genes. U.S. Patent Application Publication No. 2013/0096186 relates to a HSV vector comprising a mutant gB and/or a mutant gH glycoprotein. International Patent Application Publication No. WO 1999/06583 relates to a HSV comprising an envelope including a non-native ligand.

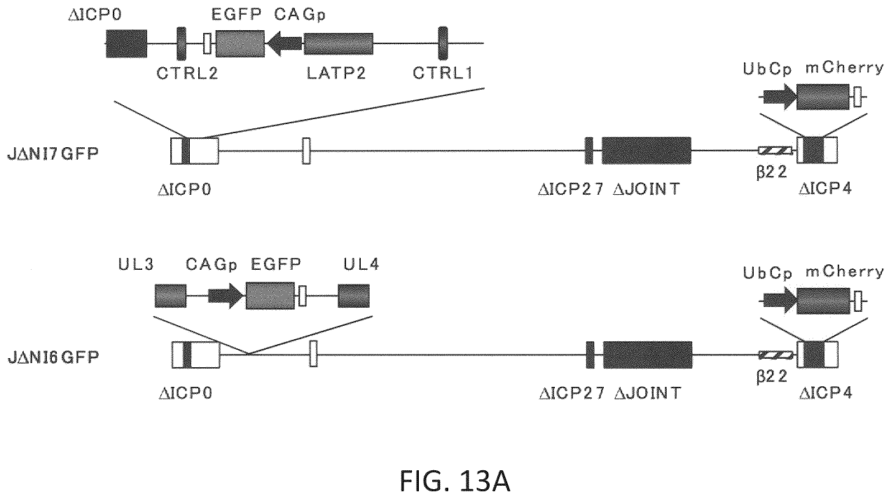

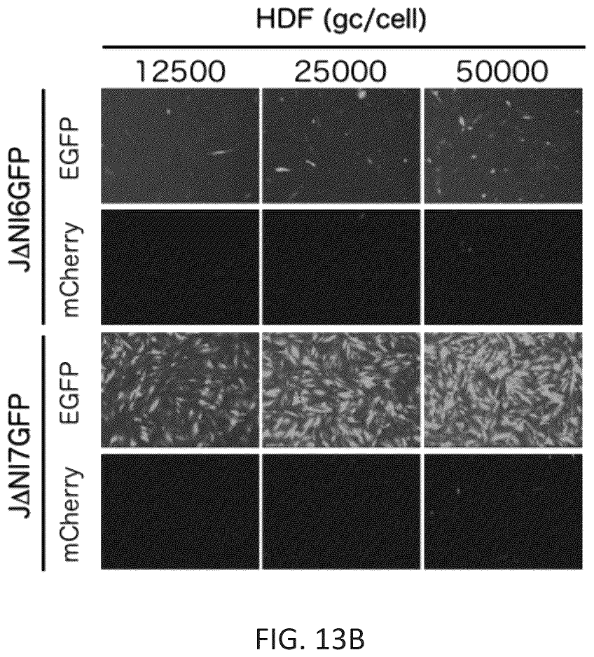

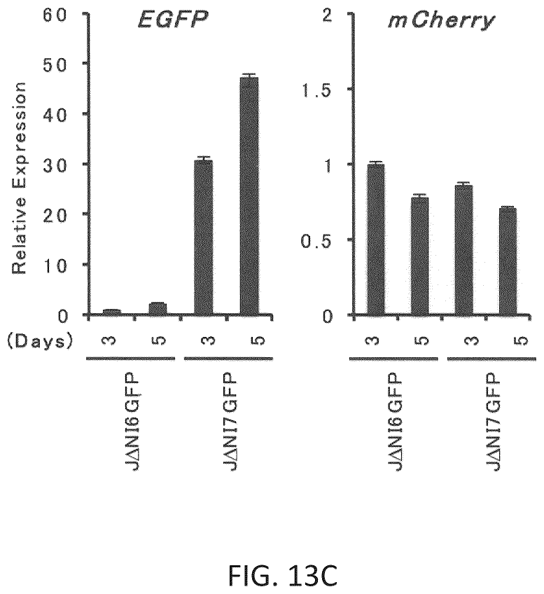

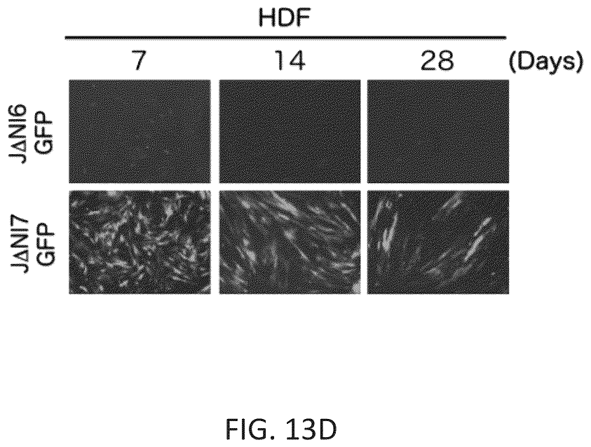

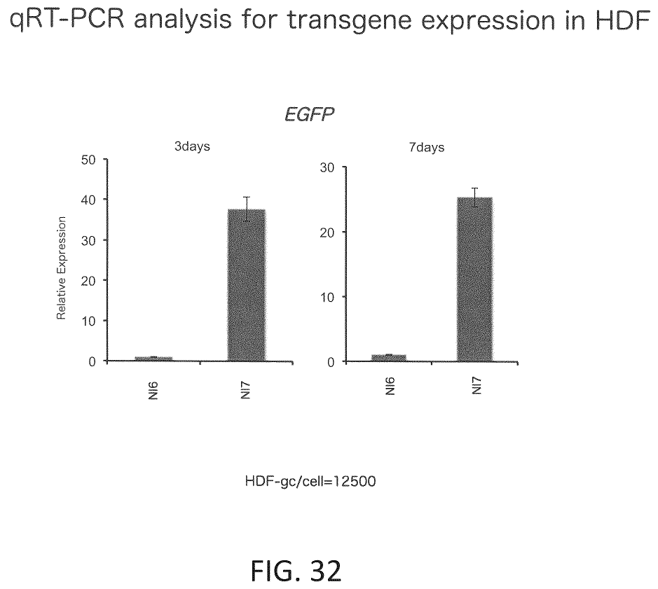

In one embodiment, the invention provides a herpes simplex virus (HSV) vector that does not express toxic native HSV genes in non-complementing cells and is capable of persistent expression of a transgene. For example, the inventive HSV vector can express a transgene for at least 14 days, such as at least 28 days, and preferably for at least 60 days, in cultured human dermal fibroblast (HDF) cells. Desirably, such expression of a transgene from the inventive vector occurs in the absence of measurable ICPO gene product within such cells, such as the absence of any ICP0 gene product within such cells. In a particular embodiment, a vector according to the present invention having a transgene (e.g., encoding Enhanced Green Fluorescent Peptide (EGFP) or another marker) inserted into the LAT region between the LAT P2 element and the CTRL2 element, and which has both CTRLI and CTRL2, can express the transgene at least 20 (or at least about 20) times the level as can a vector having otherwise identical genetic mutations but in which such a transgene is inserted between U1.3 and U1.4, as determined by quantitative RT-PCR seven days post infection and at least 30 (or at least about 30) times the level as determined by quantitative RT-PCR three days post infection. See FIGS. 13 and 32. Such vector desirably expresses such transgene at such levels and for such period of time wherein the transgene is under the operable control of a CMV enhancer/chicken beta-actin promoter/chimeric intron. That such cells can be cultured for such periods having been infected with the inventive HSV vector also is evidence that the vector is not toxic to such cells (that is, not expressing toxic native HSV genes within the cells). Of course, the non-complementing cell(s) can be another cell type as well, and can be a cell in vivo, which is of interest for therapeutic applications.

For example, the inventive HSV vector can comprise genome comprising a transgene inserted (a) within a latency-associated transcript (LAT) gene region, (b) within an ICP4 locus (and preferably only one, where the joint is deleted, as discussed herein) and/or (c) within the genome of the vector in operable connection with one or more insulator sequences within the genome. Preferably, the vector does not express ICP0, ICP4, ICP22, ICP27, and ICP47 as immediate early genes (although in some embodiments, expression of ICP47 may be desired). Without wishing to be bound by theory, it is believed that, depending on the activity of the promoter within the transgene, the inventive vector can express the transgene in any type of mammalian (especially human) cell that it can infect without the cytotoxicity associated with viral gene expression. The inventive vector can be present as isolated DNA, DNA within a cell, or packaged in a viral envelope.

Any suitable method can be employed to render the inventive vector incapable of expressing ICP0, ICP4, ICP22, ICP27, and ICP47 as immediate early genes within non-complementing cells. For example, the genome of the inventive vector can be engineered to comprise inactivating mutations (e.g., deletions) of one or all of these genes (e.g., deletions within or of the entire coding sequence of one or more of the ICP0, ICP4, ICP22, ICP27, and ICP47 genes (preferably comprising inactivating deletions of at least ICP0, ICP4, and ICP27, more preferably comprising inactivating deletions of ICP0, ICP4, ICP27 and ICP47), or also alternatively including the promoter or other regulatory sequences of such gene(s)). Alternatively, one or more of these HSV genes can be engineered to be expressed as an early or late gene. For example, the genome of certain embodiments of the inventive vector can be engineered to retain the coding sequence of one of more of these genes, but replace its promoter with one rendering the gene expressed as an early (beta) or late (gamma), but not immediate early (alpha) gene. For example, such gene can be placed under the control of a promoter responsive to ICP4 (an approach preferred at least with respect to ICP22, so as to express ICP22 as an early gene, rather than an immediate early gene). A suitable promoter for expressing such gene with early (beta) kinetics is the HSV tk promoter. The ICP22 promoter may be converted to early kinetics by truncation, i.e. deletion of regulatory sequences including TAATGARATs. The entire ICP47 promoter and initiation codon can be deleted. Alternatively, in some embodiments, the ICP47 gene can be expressed as an immediate early gene to protect infected cells against immune recognition (Hill er al, Nature 1995, 375 (6530): 411-415: Goldsmith et al, J Exp Med. 1998: 187 (3): 341-348).

In addition to the perturbation of ICP0, ICP4, ICP22, ICP27, and ICP47 expression, desirably the inventive vector also does not express UL41 (i.e., the host shut-off (vhs) gene). UL41 is an RNAse that degrades many host and viral mRNAs, causes rapid shutoff of host cell protein synthesis, and enters cells as a virion tegument component. Thus, for example, the gene encoding UL41 can be deleted from the genome of the inventive vector. Without wishing to be bound by theory, it is believed that such manipulation additionally enhances the ability of the inventive vector to grow in complementing cells by sparing the complementing ICP4 and ICP27 mRNAs, and enhances the expression of transgenes in non-complementing cells by sparing the transgene mRNAs.

It should be recognized that the genome sequences of several HSV strains are known to persons of ordinary skill (e.g., MacDonald, J. Virol. 86(11): 6371 (2012); McGeoch, J. Gen. Virol. 69:1531-1574 (1988); GenBank Accession No. JQ673480: NCBI Reference Sequence: NC_001806.1; MacDonald, J. Virol. 86 (17): 9540 (2012): GenBank Accession No. JX142173, which are incorporated herein by reference). Accordingly, manipulation of the sequence of HSV genes and loci is within the level of ordinary skill. It should also be noted that these published sequences are merely exemplary and that other strains or variants of HSV can be employed as a source genome in engineering the inventive vector.

Additionally, the genome of the inventive vector can comprise a bacterial artificial chromosome (BAC) cassette. The inclusion of such BAC cassette facilitates propagation and manipulation of the inventive vector's genome within bacteria. The BAC cassette can include bacterially-expressed sequences that assist in the use of bacterial strains, e.g., selectable genes, such as genes conferring bacterial resistance to antibiotics or toxins (e.g., preferably chloramphenicol, but other resistance genes (e.g., for tetracycline, ampicillin, zeocin, etc.) can also be employed). The BAC cassette can further include reporter genes (e.g., LacZ (encoding beta-galactosidase), or a fluorescent protein-encoding gene (e.g., gfp (encoding green fluorescent protein), yfp (encoding yellow fluorescent protein), rfp (encoding red fluorescent protein), and analogues thereof (e.g., encoding iRFP, EGFP, and the like)) under the control of a eukaryotic promoter, such as a constitutive mammalian promoter (e.g., an SV40, RSV, CMV, ubiquitin C (UbC), CAG, or β-actin promoter, etc.).

The BAC cassette can be placed into the genome of the inventive vector in any suitable location, such as the UL37-UL38 intergenic region within the vector genome (e.g., Gierash, J. Virol. Meth. 135: 197-206 (2006) and Morimoto, Microbiol. Immunol. 53:155-161 (2009)). Also, desirably, the BAC cassette is flanked by sequences facilitating removal of the BAC cassette, such as by site-specific recombinase recognition sites/consensus sequences (e.g., those recognized by enzymes such as cre, dre, flp, KD, B2, B3, R, etc.). The inclusion of such sites facilitates excision of the BAC cassette, if desired, since BAC sequences have been shown to reduce virus growth in cultured cells (e.g., Gierash, J. Virol. Meth. 135: 197-206 (2006)). Also, excision of the BAC cassette can increase the capacity for the vector to incorporate one or more transgenes, since BAC cassettes are on the order of about 11 kb. It will be appreciated that, the inventive vector also can have a consensus sequence for a recombinase enzyme (e.g., loxP), particularly one not native to the HSV genome, for example as a result of removal of a BAC cassette using a cell line that expresses an appropriate site-specific recombinase for excising the BAC cassette, as such leaves a single copy of the consensus sequences for a recombinase enzyme within the HSV genome (e.g., within the UL37-UL38 intergenic region, if sucyh was the location of a BAC cassette insert).

As noted above, the inventive vector can include at least one transgene inserted in operable connection with one or more insulator sequences within the HSV vector genome. By “operably connected.” it is to be understood that the one or more insulator sequences permit the transgene to be expressed in a cellular environment in which genetic elements (i.e., “genes”) otherwise present within the HSV genome are transcriptionally silent. Without wishing to be bound by any particular theory, it is believed that such insulator sequences prevent the formation of heterochromatin at the sites of such insulator sequences and within from about 1 kb to about 5 kb thereof, which, if formed, silences gene expression. Thus, an insulator sequence for use in the inventive vector typically is a sequence that interferes with the binding or formation of heterochromatin, which would otherwise silence expression of the transgene. Non-limiting examples of suitable insulator sequences include HSV chromatin boundary (CTRL/CTCF-binding/insulator) elements CTRL1 and CTRL2 (which can be native to the LAT locus, as described herein, or moved to an ectopic location within the genome), chicken hypersensitive site 4 insulator (cHS4), human HNRPA2B1-CBX3 ubiquitous chromatin opening element (UCOE), and the scaffold/matrix attachment region (S/MAR) from the human interferon beta gene (IFNB1) (Emery, Hum. Gene Ther. 22, 761-74 (2011): Antoniou et al., Hum. Gene Ther. 24. 363-74 (2013)).

Aside from a transgene being inserted near insulator sequences native to the HSV genome (such as the CTRL1 and CTRL2 sequences within the LAT region), insulator sequences can be inserted into the vector genome at any suitable site. These insulator/boundary elements can be introduced into the genome of the inventive vectors by standard methods, and may be included in the same cassette as a transgene, or introduced separately into the genome so as to flank or otherwise be in operable connection with a given a transgene cassette. Thus, it is possible to insert a genetic cassette including, for example, one or moer ectopic insulator sequences functionally similar to those found natively in the LAT region flanking, or otherwise in operable connection to, a transgene. It will be understood that the inventive vector can include multiple transgenes, operably linked to multiple insulator sequences, including at sites ectopic to LAT. In an embodiment in which one or more transgenes is inserted at a site other than LAT and is operably linked to CTRL1 and/or CTRL2, it is desired to delete from LAT or mutate the CTRL1 and/or CTRL2 sequences within LAT to minimize or eliminate recombination events between the native sequences within LAT and those engineered to be operably linked to a transgene within an ectopic (non-LAT) site of the inventive vector. Whether CTRL1 and CTRL2 remain within LAT or are moved ectopically, a preferred site for a transgene to be inserted into the genome of the inventive vector is between CTRL1 and CTRL2 (e.g., within about 1-4 kb of each of CTRL1 and CTRL2, wherein the CTRL1 and CTRL2 flank the transgene).

Within the inventive vector, one or more of such insulator sequences are operably linked with a transgene such that the transgene is insulated from gene silencing and is expressed. Generally, the transgene and the insulator sequence(s) should be in close proximity within the genome, such as separated by less than about 5 kb, or less than about 4 kb, or less than about 3 kb, or less than about 2 kb, or less than about 1 kb. It also can be desirable for an expression cassette (including a transgene) to be functionally between two insulator sequences such that the insulator sequences flank the transgene(s) of interest (See, e.g., Emery, Hum. Gene Ther. 22, 761-74 (2011): Antoniou et al., Hum. Gene Ther. 24, 363-74 (2013)).

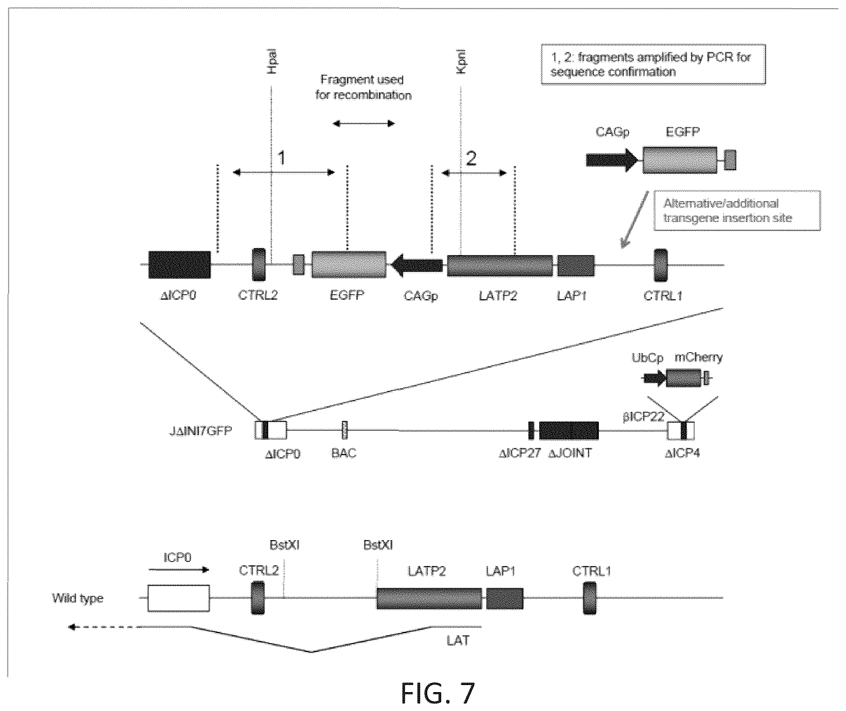

One preferred site for insertion of a transgene (e.g., a first transgene) in the inventive vectors is between insulator sequences within the LAT gene region of the vector genome-specifically inserted between chromatin boundary (CTRL/CTCF-binding/insulator) elements located upstream of the LAT promoter LAPI (CTRL1) and within the LAT 2-kb intron (CTRL2), respectively (Amelio et al, J Virol 2006, 80 (5): 2358-2368; Bloom, Biochim. Biophys. Acta. 1799:246-256 (2010)). This region is referred to herein as the LAT (gene) region or locus. Thus, desirably, the genome of the inventive vector comprises (e.g., retains) CTRL1 and CTRL2 (see FIG. 7, top). Without wishing to be bound by theory, it is believed that the presence of CTRL1 and CTRL2 protects the region against heterochromatin formation and, thus, contributes to the LAT gene region as being a privileged site for the expression of transgenes. Thus, the vector expresses the transgene inserted in the LAT gene region in non-complementing cells. In a preferred embodiment, the vector comprises a plurality of transgene cassettes within the LAT gene region, each comprising a separate promoter and coding region, and wherein each can be mono- or polycistronic.

Also, it is preferred for the LAT region of the inventive vector (which comprises at least one transgene, as noted herein) also to comprise (e.g., retain) a LATP2 or LAP2 enhancer element. Again without wishing to be bound by theory, it is believed that the presence of a LATP2 or LAP2 enhancer element contributes to the ability of the transgene within the LAT gene region to express the coding sequence(s) long-term (Goins, J. Virol., 73: 519-532 (1999): Lilley, J. Virol., 75: 4343-4356 (2001)). In a particularly preferred embodiment, the transgene(s) within the LAT gene region is inserted downstream of the LATP2 or LAP2 enhancer element. However, the invention contemplates embodiments in which the transgene(s) is inserted upstream (relative to the direction of LAT transcription) of the LATP2 or LAP2 enhancer element. Desirably, the transgene points away from the LATP2 or LAP2 elements within the LAT gene region (see, e.g., FIG. 7, top).

Another preferred site for insertion of a transgene (e.g., a second transgene) in the inventive vectors is within an ICP4 gene locus. For example, a UbC promoter-controlled transgene inserted in this locus within the inventive vector can prodice a long-term signal in hippocampal neurons and is active at least short-term in DRGs.

Transgenes can also be inserted in operable connection with (e.g., near (<5 kb)) other insulator sequences within the genome of the inventive vectors. Aside from being inserted near insulator sequences native to the HSV genome, insulator sequences can be inserted into the vector genome at any suitable site. Thus, it is possible to insert a genetic cassette including, for example, ectopic insulator sequences functionally similar to those found natively in the LAT region flanking a transgene. It will be understood that the inventive vector can include multiple transgenes.

Within the transgene(s) inserted into the inventive vector, there are at least a promoter sequence and a transcribed sequence such that the transcribed sequence(s) is controlled by the promoter. The promoter within the transgene can be any promoter desired to control/regulate the expression of the transcribed sequence(s). For example, the promoter can be a cell-specific or tissue-specific promoter (e.g., EOS, OCT4, Nanog (for ESC/iPSC), SOX2 (for neural stem cells), αMHC, Brachyury, Tau, GFAP, NSE, Synapsin I (for neurons), Apo A-I, Albumin, ApoE (for liver), MCK, SMC α-Actin, Myosin heavy chain, Myosin light chain (for muscle), etc.), such as a promoter that specifically or preferentially expresses genes in a defined cell type (e.g., within a liver cell, lung cell, epithelial cell, cardiac cell, neural cell, skeletal muscle cell, embryonic, induced pluripotent, or other stem cell, cancer cell, etc.). Preferred promoters for use in sensory neurons include TRPV1, CGRP. and NF200. In other embodiments, the promoter within a transgene inserted into the inventive vector can be an inducible promoter (e.g., TRE3G combined with rtTA3G expression from a separate promoter in LAT or other inducible promoters as are known in the art). Of course, the promoter within a transgene expression cassette inserted into the inventive vector can be a constitutive mammalian promoter, such as are known in the art (e.g., SV40, CMV, CAG, EF1α, UbC, RSV, β-actin, PGK, and the like).

In addition to the promoter(s) and coding sequence(s), the transgene(s) inserted into the genome of the inventive vector also can comprise additional regulatory element(s). For example, the transgene(s) can include one or more sites for binding of microRNA. In preferred embodiments, the transgene(s) comprise tandem binding sites for such microRNAs, such as 2, 3, 4, 5, or 6 tandem sites (four being typical). The presence of such sites, particularly tandem binding sites for such microRNAs, facilitates down-regulation of the transgene expression in certain cell types. Thus, for example, a vector comprising a transgene desired to be expressed in a cancer or tumor cell (which may be toxic to many cell types) can comprise binding sites for microRNAs of “normal” (i.e., non-malignant) cells, so that the expression of the transgene is suppressed in non-malignant cells.

It should be noted that the transgene(s) within the inventive vector can be monocistronic (i.e., encoding a single protein or polypeptide) or polycistronic (i.e., encoding multiple proteins or polypeptides). Moreover all or part of the transcribed portion of the transgene also can encode non-translated RNA, such as siRNA or miRNA, for example. Also the inventive vector can comprise multiple separate monocistronic or polycistronic transgene units (preferably two separate transgene units but possibly more (e.g., three, four, five, or more separate units)), each with its own respective promoter, translated sequence(s) or non-translated RNA sequence(s), and other regulatory elements.

As noted, the transgene(s) include(s) one or more transcribed sequence(s), which are expressed under the control of the promoter and optionally other regulatory elements within the transgene (including the operable connection to the insulator sequence(s)). A transcribed sequence can be any sequence desired to be expressed within a given cell into which the vector is to be introduced. Non-limiting examples of transcribed sequences that can be present in a transgene within the inventive vector include Oct4, Klf4, Sox2, c-Myc, L-myc, dominant-negative p53, Nanog, Glis1, Lin28, TFIID, GATA4, Nkx2.5, Tbx5, Mef2C, Myocd, Hand2, SRF, Mesp1, SMARCD3, SERCA2a, Pax3, MyoD, Lhx2, FoxG1, FoxP2, Isl1, Ctip2, Tbr1, Ebf1, Gsx2, Srebp2, Factor VIII, Factor IX, Dystrophin, CFTR, GlyRα1, enkephalin, GAD67 (or other GAD isoforms, e.g., GAD 65), TNFα, IL-4, a neurotrophic factor (e.g., NGF, BDNF, GDNF, NT-3), Ascl1, Nurr1, Lmx1A, Bmn2, Myt1, NeuroD1, FoxA2, Hnf4α, Foxa1, Foxa2 or Foxa3, any microRNA or combination of miRNAs (e.g., hsa-mir-302/367 gene cluster: hsa-miR200c; hsa-miR369; hsa-mir-124) and/or one or more other non-coding RNAs (“ncRNA(s)”) or a reporter gene for expression in mammalian cells, such as LacZ (encoding beta-galactosidase), CAT (encoding chloramphenicol acetyltransferase), or a fluorescent protein-encoding gene (e.g., gfp, yfp, rfp, and analogues thereof such as iRFP, EGFP, and the like).

In addition to the foregoing, the inventive vector optionally also can comprise an expression cassette inserted in a site other than the LAT region or other than in the vicinity of known insulator sequences. A preferred site for such expression cassette is ICP4. For example, when the vector comprises a complete or inactivating deletion of the ICP4 gene, the expression cassette can be inserted into the site of the ICP4 deletion. Desirably, the coding sequence of the expression cassette inserted into a site other than the LAT region is controlled by a constitutive mammalian promoter (e.g., SV40, CMV, CAG, EF1α UbC, RSV, β-actin, PGK, and the like), but other promoters (as discussed herein and otherwise known in the art) can be used, if desired. One exemplary expression cassette includes UbCp driving expression of mCherry, engineered into a deleted ICP4 locus. Of course, such a transgene can encode a factor of therapeutic interest as well.

In addition to the foregoing, the inventive HSV vector also desirably comprises a deletion of the internal repeat (Joint) region, comprising IRₛ and IR_{L}. Deleting this region can contribute to the stability of the vector genome, and deleting this sequence of HSV DNA also allows for the vector to accommodate large transgenes (at least 15 kb) and still be packaged correctly into mature virions. Deletion of the Joint eliminates one copy each of the IE genes ICP0 and ICP4 such that the remaining copies can be easily manipulated. It also deletes the promoter for the ICP22 or ICP47 immediate early gene. If desired, expression of the ICP47 gene can be restored by insertion of an immediate early promoter, preferably the ICP0 promoter or the HCMV major IE promoter, to minimize immune recognition of infected cells (Hill er al, Nature 1995, 375(6530): 411-415; Goldsmith et al. J Exp Med. 1998: 187(3): 341-348).

HSV is able to infect a wide variety of mammalian cells: thus, the inventive vector has broad applicability. However, to enhance infectivity, desirably the envelope of the inventive vector can further include a mutant glycoprotein that enhances infection and/or lateral spread relative to a wild-type glycoprotein. Alternatively or in addition, the envelope of the inventive vector can further include a mutant glycoprotein that directs HSV entry into cells through non-canonical receptors. For example, such mutant glycoprotein(s) can be gB, gC, gD. gH, or gK: of course, the vector can have more than one such mutant (enhanced-penetration or-spread) glycoproteins (e.g., a combination of two, more, or even all thereof). Furthermore, technology for mutating such glycoproteins to enhance HSV infection and/or lateral spread is known, and any such technology can be employed in the context of the present invention (see, e.g., U.S Patent Application Publication No. 2013-0096186 A1; International Patent Application Publication No. WO/1999/006583, Uchida, J. Virol., 84: 12200-12209 (2010), Uchida et al., J. Virol., 87(3). 1430-42 (2013), and Uchida, Mol. Ther., 21: 561-569 (2013), which are incorporated herein by reference). Moreover, the genome of the inventive vector can comprise a mutant gene encoding such mutant glycoprotein(s).

Exemplary vector “backbones” embodying the invention are described herein as “JΔI5” and “JΔI8” with the understanding that, as “backbones,” it is contemplated that transgenes with or without extraneous control elements can be inserted into the LAT region of these specific vectors (see, e.g., FIG. 7, top).

One application of the inventive vectors is the reprogramming of a variety of cell types to produce pluripotent stem cells. Stem cells have been at the forefront of biomedical research in recent years and hold a great deal of promise for understanding human development, genetic disease and the creation of new therapies in regenerative medicine. In 2006, Yamanaka and colleagues discovered a means of creating embryonic-like stem cells by regrogramming adult fibroblasts (Takahashi and Yamanaka, Cell 126: 663-76, 2006). These novel cells, designated induced pluripotent stem (iPS) cells, are functionally similar to ES cells (Wernig et al, Nature 448: 318-24, 2007) and when derived from human somatic cells (Takahashi et al, Cell 131: 861-72, 2007: Yu et al, Science 318: 1917-20, 2007), sidestep the ethical concerns involving the use of human ES cells. Four reprogramming genes were originally employed for iPS cell production (Takahashi and Yamanaka, Cell 126: 663-76, 2006; Takahashi et al, Cell 131: 861-72, 2007, but the efficiency of reprogramming with these or other genes has remained problematic due to inefficient gene transfer methods. The inventive vector system addresses this issue by combining high transduction efficiency for many cell types with the capability of expressing multiple transgenes simultaneously from a single vector. For example, the JΔI7 and JΔI8vectors described herein are replication-defective and non-toxic due to deletion or altered expression kinetics of the five viral IE genes. Since they do not integrate into the cellular genome, such vectors are diluted during cell division, providing a hit-and-run gene delivery system.

To facilitate growing, producing, and propagating the inventive vectors and producing stocks thereof, an aspect of the invention provides a complementing cell line, which complements ICP0 and ICP4, desirably ICP0, ICP4 and ICP27. Preferably, the ICPO complementation is achieved without expressing HSV ICP0, to reduce toxicity within the complementing cell. Thus, a preferred complementing cell according to the present invention is derived from a cell type that naturally complements HSV ICP0 function, such as U2OS cells (Yao, J. Virol., 69: 6249-6258 (1995), which is incorporated herein by reference). Such cells can be engineered to express ICP4 or ICP4 and ICP27 by methods known in the art (e.g., by introducing ICP4 or ICP4 and ICP27 expression cassettes within the cells so that they express ICP4 and ICP27, respectively, from genetic constructs other than the HSV genome, such as the cellular chromosomes). Desirably, the cell line expresses ICP4 and ICP27, respectively, in trans. Desirably, the introduced ICP4 or ICP4/ICP27 coding sequences are under the control of their cognate viral promoters. Also, one or both of the ICP4 and ICP27 complementing coding sequences can be inducibly expressed within such cells upon infection with HSV.

As noted above, embodiments of the inventive vector comprise a BAC flanked by sequences facilitating removal of the BAC cassette by a site-specific recombinase recognition. Accordingly, the inventive complementing cell can be engineered to further express a gene encoding a site-specific recombinase appropriate to the recognition sequences within the vector, thereby producing the recombinase protein. Thus, the inventive complementing cell can express and produce cre, dre, flp, KD, or B2, B3, R, and the like, or a mutant derivative thereof, as appropriate. Passage through such a cell line, thus, recovers room for transgenes (about 11 kb) and can improve virus growth by over 10 fold, such as over 25 fold, or over 50 fold, such as about 100 fold. The improved viral growth can be assayed by standard procedures (to produce growth curves) in comparison with cells that lack the recombinase. This involves infection of replicate wells of cells at low MOI, virus collection at different times post infection, and titration of the yields typically by plaque assay.

Additionally, the complementing cell line can be engineered to express a gene encoding a selectable marker, such as markers typically employed in engineering packaging cells or cells expressing any other foreign gene. Suitable selectable genes include those conferring resistance to neomycin/G418, hygromycin, blasticidin, puromycin, zeocin, and the like.

It will be understood that methods for engineering a source cell type (e.g., U2OS cells) to contain expression constructs encoding the HSV ICP4 and ICP27 proteins, as well as other proteins (such as the recombinase and/or the selectable gene product) are known to persons of ordinary skill. For example, the gene of interest with a selectable marker can be subcloned into lentiviral vectors, the source cell infected with the lentiviral vectors, selected for expression of the marker (e.g., blasticidin resistance), and then expression of the transgene of interest (e.g., HSV ICP27) confirmed.

Of course, the inventive complementing cell can be propagated and cloned. Thus, the invention provides a clonal population, i.e., a cell line, comprising or consisting of or essentially of the complementing cell line as described herein.

Using the inventive complementing cells of the invention, the inventive HSV vector can be propagated. Accordingly, the invention provides a method of propagating the HSV vector of the present invention. In accordance with the inventive method, the complementing cell line is transfected with vector DNA and then cultured until plaques form. The viral DNA, as noted, can have a BAC, and, if so, then the inventive cell can express the recombinase appropriate for excising the BAC from the viral genome, if it is desired not to include the BAC in the packaged vector. The viral population is amplified by repeated transfer of infectious particles to increasingly large, fresh populations of the complementing cells. For these repeated transfers, multiplicity of infection (MOI) can be between about 0.001 pfu/cell and about 0.03 pfu/cell. Ultimately, the inventive vectors (as packaged viruses) are purified from the cells at 90% cytopathic effect.

Generally, the inventive HSV vector is most useful when enough of the virus can be delivered to a cell population to ensure that the cells are confronted with a suitable number of viruses. Thus, the present invention provides a stock, preferably a homogeneous stock, comprising the inventive HSV vector. The preparation and analysis of HSV stocks is well known in the art. For example, a viral stock can be manufactured in roller bottles containing cells transduced with the HSV vector. The viral stock can then be purified on a continuous nycodenze gradient, and aliquotted and stored until needed. Viral stocks vary considerably in titer, depending largely on viral genotype and the protocol and cell lines used to prepare them. Preferably, such a stock has a viral titer of about 10⁶ pfu/ml or even more preferably about 10⁷ pfu/ml (or at least about such values). In still more preferred embodiments, the titer can be about 10⁸ pfu/ml, or about 10⁹ pfu/ml (or at least about such values), and high titer stocks of about 10¹⁰ pfu/ml or about 10¹¹ pfu/ml or even about 10¹² pfu/ml (or at least about such values) are most preferred. Thus, the titer of an HSV stock according to the present invention can vary from about 10⁶ pfu/ml to about 10¹² pfu/ml (preferably between about 10⁹ to about 10¹¹ pfu/ml). Genome copy (gc) numbers provide a cell line-independent measure of the number of virus particles, but include defective particles. Typically gc values for wild-type HSV-1 are several to 20×, up to 100×, higher than the pfu values of the same stocks. For mutant viruses, especially defective viruses grown on complementing cells, this can increase to 10,000× higher or even more. Gc and pfu values increase proportionally with the size of the stock.

The invention additionally provides a composition comprising the HSV vector and a carrier, preferably a physiologically-acceptable carrier. The carrier of the composition can be any suitable carrier for the vector. The carrier typically will be liquid, but also can be solid, or a combination of liquid and solid components. The carrier desirably is a pharmaceutically acceptable (e.g., a physiologically or pharmacologically acceptable) carrier (e.g., excipient or diluent). Pharmaceutically acceptable carriers are well known and are readily available. The choice of carrier will be determined, at least in part, by the particular vector and the particular method used to administer the composition. The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use.

Extemporaneous injection solutions and suspensions can be prepared from sterile powders, granules, and tablets of the kind previously described.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the vector and physiological distress. Immune system suppressors can be administered with the composition method to reduce any immune response to the vector itself or associated with a disorder. Alternatively, immune enhancers can be included in the composition to up-regulate the body's natural defenses against disease. Antibiotics, i.e., microbicides and fungicides, can be present to reduce the risk of infection associated with gene transfer procedures and other disorders.

Using the inventive vector (and stocks and compositions comprising the vector), the invention provides a method of expressing a transgene within a nucleated cell, especially a non-complementing cell. In accordance with the method, the inventive vector is exposed to the cell under conditions suitable for the vector to infect the cell. Once the cell is infected, the transgene inserted within the vector's LAT region will be transcribed (expressed) within the cell, provided the promoter within the transgene is one which is active in the cell and that the transgene is not suppressed by another regulatory mechanism (e.g., the microRNAs discussed herein). In other words, the inventive vectors serve as gene transfer and expression vectors within mammalian cells.

The inventive method can be employed to express transgene(s) within cells either in vivo or in vitro, as desired. For use in vivo, the cell can be any type of desired cell, such as exocrine secretory cells (e.g., glandular cells, such as salivary gland cells, mammary gland cells, sweat gland cells, digestive gland cells, etc.), hormone secreting gland cells (e.g., pituitary cells, thyroid cells, parathyroid cells, adrenal cells, etc.), ectoderm-derived cells (e.g., keratinizing epithelial cells (e.g., making up the skin and hair), wet stratified barrier epithelial cells (e.g., of the cornea, tongue, oral cavity, gastrointestinal tract, urethra, vagina, etc.), cells of the nervous system (e.g., peripheral and central neurons, glia, etc.)), mesoderm-derived cells, cells of many internal organs (such as kidney, liver, pancreas, heart, lung) bone marrow cells, and cancerous cells either within tumors or otherwise. Preferred, and non-limiting examples of cells suitable for infection by the inventive vectors include liver cells, lung cells, epithelial cells, cardiac cells, muscle cells, stem cells, and cancer cells.

It will be observed that, when used in vivo, the inventive method can treat a disease or a condition within a subject, when the transgene within the vector encodes one or more prophylactically-or therapeutically-active proteins, polypeptides, or other factor (e.g., non-coding RNA (ncRNA) such as siRNA or miRNA). Thus, the invention provides a method of treating a disease or condition in a subject, comprising administering the vector of the present invention to the subject, in an amount and at a location sufficient to infect cells of the subject such that the transgene is expressed within the cells of the subject, and wherein the transgene encodes one or more prophylactically or therapeutically active proteins, polypeptides or ncRNA. For example, the disease or condition can be a type of cancer, in which the transgene can encode an agent that enhances tumor killing activity (such as TRAIL or tumor necrosis factor (TNF)). As additional non-limiting example, the transgene can encode an agent suitable for the treatment of conditions such as muscular dystrophy (a suitable transgene encodes Dystrophin), cardiovascular disease (suitable transgenes include, e.g., SERCA2a, GATA4, Tbx5, Mef2C, Hand2, Myocd, etc.), neurodegenerative disease (suitable transgenes include, e.g., NGF, BDNF, GDNF, NT-3, etc.), chronic pain (suitable transgenes encode GlyRα1, an enkephalin, or a glutamate decarboxylase (e.g., GAD65, GAD67, or another isoform), lung disease (e.g., CFTR), or hemophilia (suitable transgenes encode, e.g., Factor VIII or Factor IX).

In other embodiments, the inventive method can be used in vitro to cause expression of the transgene within cells in culture. Again, any type of cells can be infected in vitro with the inventive method, such as stem cells and fibroblasts, such as a human dermal fibroblast (HDF) or a human lung fibroblast (HLF). Other preferred types of cells for use in vitro include keratinocytes, peripheral blood mononuclear cells, hematopoietic stem cells (CD34+), or mesenchymal stem/progenitor cells. In one embodiment, the transgene(s) encode one or more factors that can effect the differentiation of the cell. For example. expression of one or more of Oct4, Klf4, Sox2, c-Myc, L-Myc, dominant-negative p53, Nanog, Glis1, Lin28, TFIID, mir-302/367, or other miRNAs can cause the cell to become an induced pluripotent stem (iPS) cell. See also, Takahashi and Yamanaka, Cell, 126:663-676(2006): Takahashi, Cell, 131:861-872 (2007): Wernig, Nature. 448:318-324 (2007): and Yu, Science. 318: 1917-1920 (2007), the disclosures of which are incorporated herein by reference. Alternatively, the transgene(s) within the inventive vectors can encode a factor for transdifferentiating the cells (e.g., one or more of GATA4, Tbx5, Mef2C, Myocd, Hand2, SRF, Mesp1, SMARCD3 (for cardiomyocytes); Ascl1, Nurr1, Lmx1A, Br2, Myt1l, NeuroD1, FoxA2 (for neural cells), Hnfα, Foxa1, Foxa2 or Foxa3 (for hepatic cells)

In practicing the inventive method involving infecting a cell in vivo or in vitro with the vector, composition, or stock of the invention, the cell can be any mammalian nucleated cell for which it is desired to express the transgene. HSV has broad infectivity and. as noted herein, the inventive vector can be engineered to alter its natural tropism and to enhance infectivity by mutating viral envelope glycoproteins. Thus, the vector can be employed to infect cells of many mammalian species. It is believed that the inventive methods can be applied in agriculture, such as to express exogenous genes or supplement for deficient genes in animals such as cattle, horses, sheep, goats, swine, and the like. Similarly, the inventive method can be employed in a veterinary context for companion animals, such as cats, dogs, and the like.

Of course, the inventive method can be used in vivo in humans as well, to provide for the expression of a prophylactically-or therapeutically-active agent, or factor, in a medical setting. The factor (supplied by expression of one or more of the transgenes within the inventive vectors) can be exogenous, or one that complements a genetic deficiency.

The following examples further illustrate the invention but, of course, should not be construed as in any way limiting its scope.

### EXAMPLE 1

This example describes the development of a complementing cell line for replicating and producing the inventive HSV vectors.

Several of the immediate early (IE) genes of HSV are essential for virus replication, but these and other IE genes have toxic effects in a variety of cell types. While the removal of these genes prevents vector toxicity, the essential products must be provided in order to produce infectious viral particles. A novel cell line, based on U2OS human osteosarcoma cells, was engineered to conditionally express the essential IE genes ICP4 and ICP27. These genes were introduced under the control of their cognate viral promoters by retrovirus-mediated insertion. As such these genes may remain silent until HSV infection delivers the HSV tegument protein VP16 to the nucleus where it promotes high-level expression of the integrated ICP4 and ICP27 genes by activation of their promoters. Thus, prior to HSV infection, these genes can be stably maintained in the engineered U2OS cells without unsuitable toxicity. HSV growth also relies on the expression of ICP0, but this protein inhibits cell replication, causing cell cycle arrest and programmed cell death. Remarkably, U2OS cells naturally complement ICP0 functions and thus the introduction of ICP4 and ICP27 is sufficient to provide a cellular environment for efficient production of vectors deleted for all three IE genes. This novel engineered cell line is stable, grows well in culture and can be used for clinical production of non-toxic HSV vectors.

U2OS-ICP4/27 cells and 7b (Vero-ICP4/ICP27) cells were infected with E1G6 (ΔICP4::HCMVp-eGFP/ΔICP27/β22/β47) or JDQOZEH1

(ΔICP4/ΔICP27/ΔICP22/AICP0::HCMV-eGFP) viruses. After 3 days, virus in the cell supernatants were titered on U2OS-ICP4/27 cells.

| | Production | Titer | Fold Difference |
|---|---|---|---|
| Virus | Cell Line | (PFU/ml) | (U2OS-ICP4/27 vs 7b) |
| E1G6 | U2OS-ICP4/277b | 2.9 × 10⁵ | 328 × decrease |
| | | 9.5 × 10⁷ | |
| JDQOZEH1 | U2OS-ICP4/277b | 2.6 × 10⁵ | 2600 × increase |
| | | 1.0 × 10² | |

The data show that E1G6 (ICP4- and ICP27-deleted; no expression of ICP22 and ICP47) grew on both cell lines whereas JDQOZEH1 (deleted for ICP0, ICP4, ICP27 and ICP22) was only able to grow on U2OS-ICP4/27 cells. The JDQOZEHI titer in 7b supernatant likely represents residual input from the 7b infection.

### EXAMPLE 2

This example describes an embodiment of a HSV vector comprising a genome comprising a transgene inserted within a LAT gene region, wherein the vector does not express ICP0, ICP4, ICP22, ICP27, and ICP47 as immediate early genes.

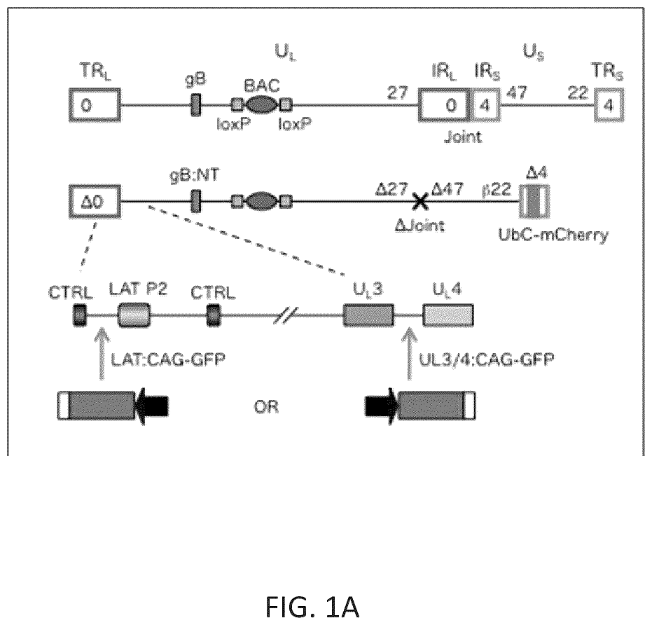

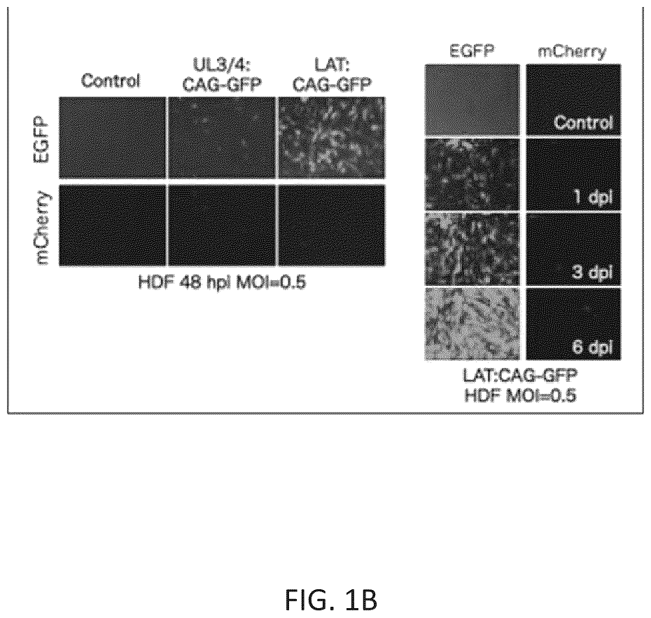

The vector genome (FIG. 1A) contains a Cre-removable bacterial artificial chromosome (BAC) cassette in the UL37-UL38 intergenic region allowing propagation and manipulation in bacteria. It is deleted for the non-essential internal repeat region (Joint, 14 kb) separating the U_{L} and Uₛ segments to provide space for transgene insertion and increase vector stability. The vector is replication defective due to deletion of the ICP4 and ICP27 genes, and is additionally deleted for the toxic IE gene ICP0 and the promoter and start codon of the ICP47 IE gene; the remaining toxic IE gene, ICP22, is controlled by an ICP4-dependent (early, β) promoter, so as to express ICP22 during the “early,” rather than the “immediate early,” phase of HSV gene expression in ICP4-complementing cells. Because U2OS cells naturally complement ICP0, the vector was grown in a U2OS-based virus producer cell line, U2OS-ICP4/ICP27, that complements the functions of all of these IE genes except that of the dispensable ICP47 gene. The vector genome also contains a pair of mutations in the gB gene that enhance virus entry into cells and a ubiquitin (UbC) promoter-mCherry reporter gene expression cassette at the position of the ICP4 deletion. In non-complementin cells, the latency-associated transcript (LAT) promoter region, which is located between insulator (CTRL) elements that protect the region against heterochromatin formation remains active. This region in addition contains an enhancer element, LAT P2 or LAP2, that promotes long-term gene expression. A CAG promoter-GFP expression cassette was inserted between LATP2 and CTRL2 and robust GFP expression was observed in infected human dermal fibroblast (HDF) cells whereas minimal expression was observed from the same GFP cassette inserted at other locations in the genome or from the mCherry cassette (FIG. 1B). Thus, in the complete absence of IE gene expression, the LAT locus is a privileged site for transgene expression.

FIG. 1A shows structures of the complete HSV-1 genome with the BAC sequences in UL (top), and the base vector construct. The LAT and UL3-UL4 regions are enlarged underneath and the alternative positions of CAG-GFP insertion are indicated. FIG. 1B shows GFP expression from alternative positions in the vector genome in infected HDFs. The GFP gene within this vector encodes EGFP.

### EXAMPLE 3

This example lists the structure and properties of various HSV vector constructs.

| | | | | | | | | | HDF | mCherry | GFP |
|---|---|---|---|---|---|---|---|---|---|---|---|
| vector feature −> | ICP 0 | ICP 4* | ICP 22 | ICP 27 | ICP 47 | LAT | UL₄₁ | UL₃₋₄ | toxicity | expression** | expression** |
| Virus | | | | | | | | | | | |
| JΔβββ3 | β | Δ | β | β | Δp | wt | Wt | wt | ++++ | + | n/a |
| JΔNI3 | Δ | Δ | β | β | Δp | wt | Wt | wt | +++ | − | n/a |
| JΔβββ4 | β | Δ | β | β | Δp | wt | Wt | EF1α-GW | ++++ | + | n/a |
| JΔNI4 | Δ | Δ | β | β | Δp | wt | Wt | EF1α-GW | +++ | − | n/a |
| JΔβββ4-OKS | β | Δ | β | β | Δp | wt | ¹OKS | EF1α-GW | | + | n/a |
| JΔβββ4-OKSM | β | Δ | β | β | Δp | wt | ¹OKS | EF1α-MYC | ++ | + | n/a |
| JΔNI4-OKS | Δ | Δ | β | β | Δp | wt | ¹OKS | EF1α-GW | | − | n/a |
| JΔNI4-OKSM | Δ | Δ | β | | Δp | wt | ¹OKS | EF1α-MYC | + | − | n/a |
| JΔNI5 | Δ | Δ | β | Δ | Δp | wt | Wt | wt | + | − | n/a |
| JΔNI6 | Δ | Δ | β | Δ | Δp | wt | Wt | EF1α-GW | = | − | n/a |
| JΔNI6-OKSM*** | Δ | Δ | β | Δ | Δp | wt | ¹OKS | EF1α-MYC | ? | ? | n/a |
| JΔNI6-EF1αGFP | Δ | Δ | β | Δ | Δp | wt | Wt | EF1α-GFP | + | − | − |
| JΔNI6-CAGGFP | Δ | Δ | β | Δ | Δp | wt | Wt | CAG-GFP | + | − | − |
| JΔNI7-GFP | Δ | Δ | β | Δ | Δp | CAG-GFP | Wt | wt | + | − | +++ |
| JΔNI7-OKSM | Δ | Δ | β | Δ | Δp | CAG-OKSM | Wt | wt | ? | ? | n/a |
| JΔNI7-EF1α2GFP | Δ | Δ | β | Δ | Δp | ²EF1α2-GFP | Wt | wt | + | − | + |
| JΔNI7- | Δ | Δ | β | Δ | Δp | ²EF1α2- | Wt | wt | ? | ? | ? |
| miR302GFP | | | | | | miR302/367- | | | | | |
| | | | | | | GFP | | | | | |
| JΔNI8 | Δ | Δ | β | Δ | Δp | wt | Δ | wt | − | − | n/a |
| JΔNI8-GFP | Δ | Δ | β | Δ | Δp | CAG-GFP | Δ | wt | − | + | +++ |

^{□}Δ = deletion;
β = converted to early expression kinetics;
Δp = promoter and start codon deleted
¹CAG promoter
²Contains EF1α first intron following the EF1α promoter
*All have the same mCherry expression construct in the deleted ICP4 locus
**In HDF (human dermal fibroblasts) at 3 dpi

### EXAMPLE 4

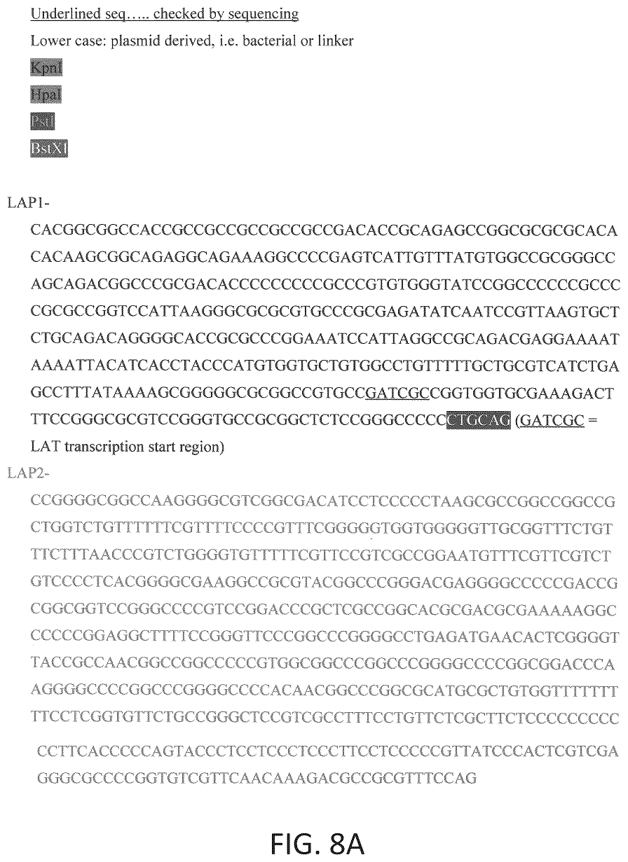

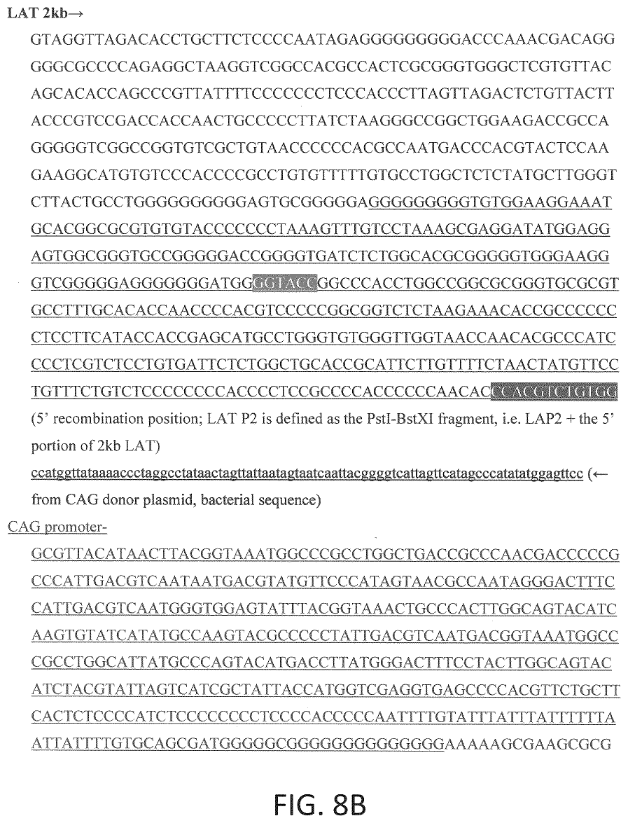

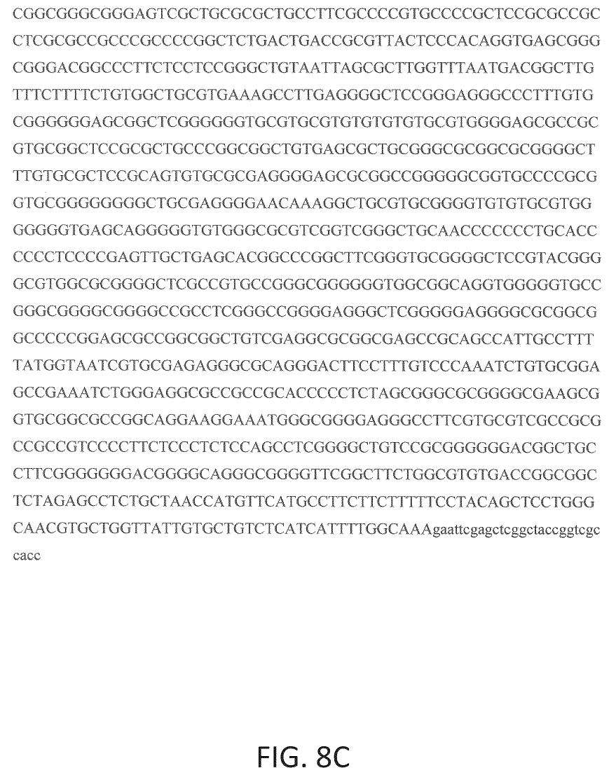

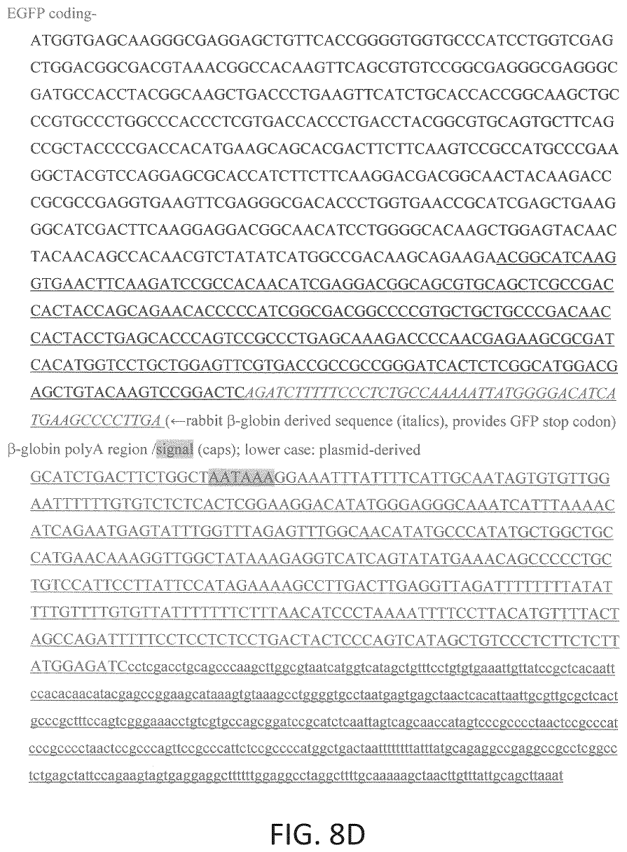

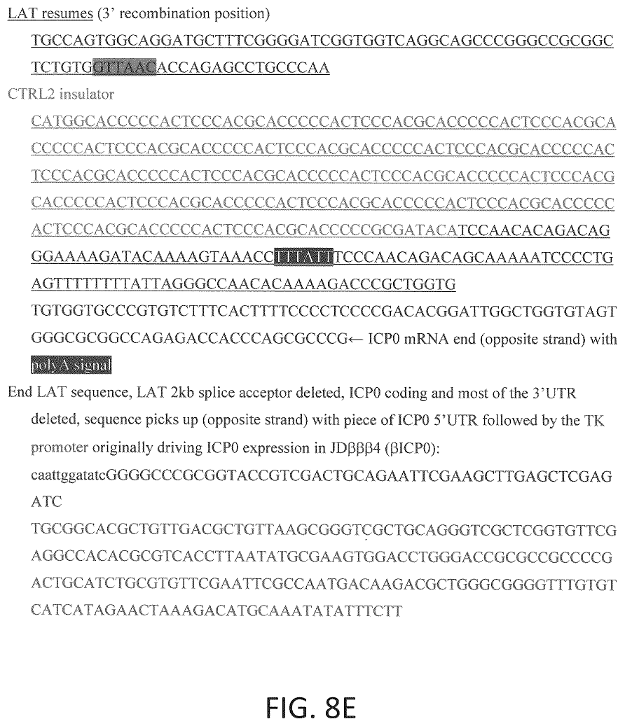

This example demonstrates the transgene expression in JΔNI7-GFP-and JΔNI6-CAGGFP-infected cells. The location of CAG-GFP in JΔNI7-GFP is shown in FIG. 1 as LAT:CAG-GFP: its location in JΔNI6-CAGGFP is shown in FIG. 1 as UL3/4:CAG-GFP. Furthermore, the sequence of the LAT region of JΔNI7-GFP HSV is set forth in FIGS. 8A-8E (SEQ ID NO:1), including the sequences of various genetic elements within the LAT region. It will be noted that the GFP within these vectors encodes EGFP.

The biological titer of the virus stock was determined on U2OS-ICP4/ICP27 cells and the genome copy (gc) titer was determined by quantitative real-time PCR for the viral glycoprotein D gene. The particle (gc)-to-plaque forming units (PFU) ratios for JΔNI6-CAGGFP and JΔNI7-GFP were comparable. See Example 8, table 2.

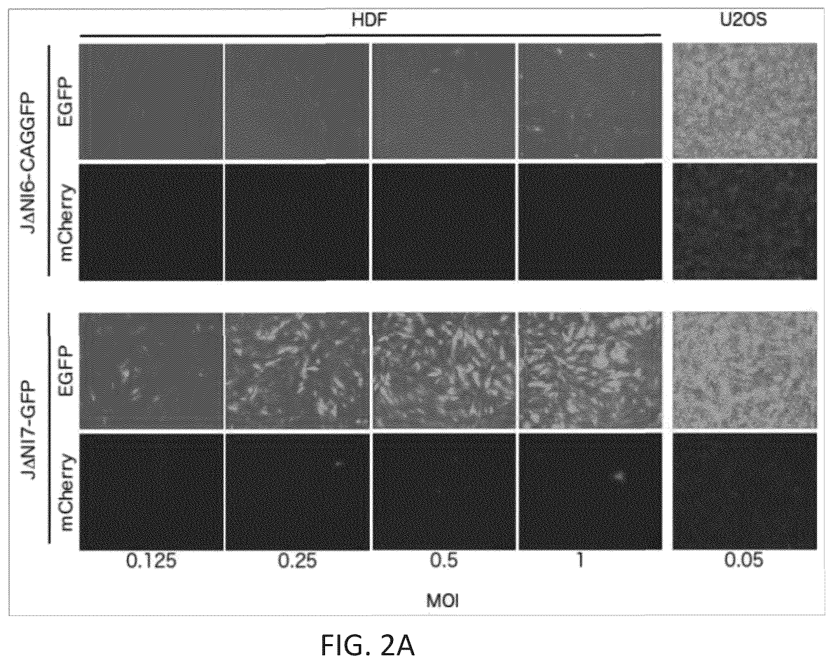

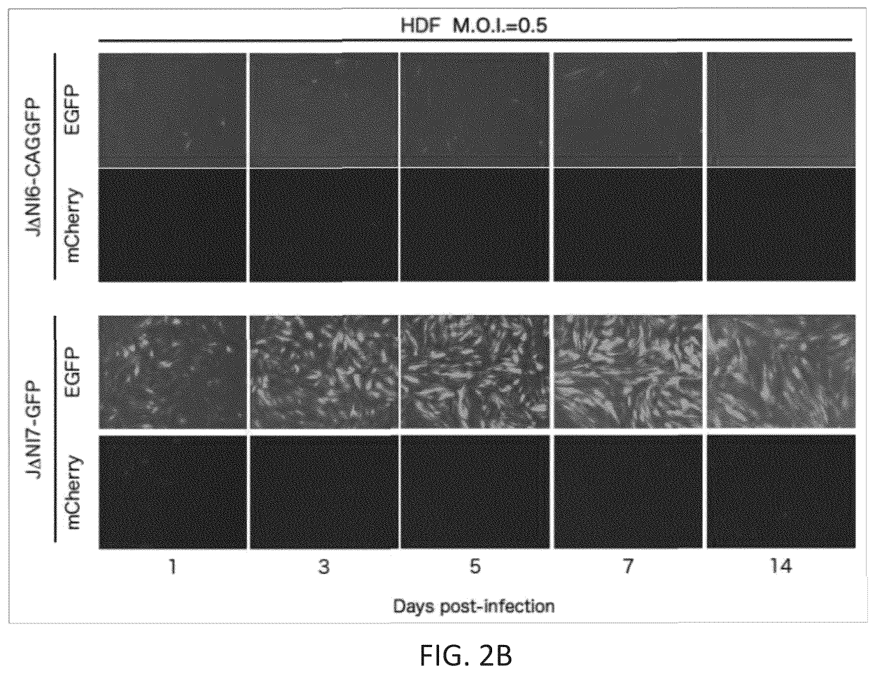

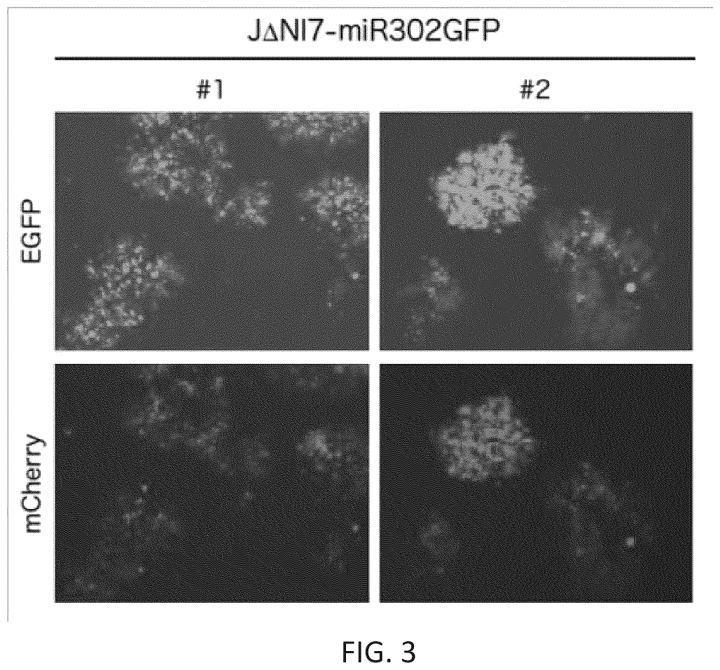

Non-complementing human dermal fibroblasts (HDF) and ICPO-complementing U2OS cells were infected with each virus in order to compare their transgene expression. JΔNI7-GFP, containing the CAG-GFP cassette in the LAT locus, showed strong, viral dose-dependent GFP expression in HDF whereas the highest dose of JΔNI6-CAGGFP yielded only minimal GFP expression (FIG. 2A, left panels, EGFP). GFP expression in JΔNI7-GFP-infected HDF remained detectable 2 weeks after infection (FIG. 3B, EGFP). However, little or no mCherry expression was observed from either virus in HDF (FIG. 2A, 2B, mCherry), suggesting that genes outside the LAT locus are silenced in HDF. In contrast, abundant GFP and mCherry expression was observed for both viruses at low MOI in U2OS cells (FIG. 2A, right column), consistent with the interpretation that the IC0O-like activity of these cells prevents the silencing of non-LAT loci occurring in HDF. Together, these results strongly indicated that the LAT locus is a preferred site for transgene expression from replication-defective, ICP0-deficient vectors.

### EXAMPLE 5

This example demonstrates the production of two isolates of JΔNI7-miR302GFP BAC in U2OS-ICP4/ICP27 cells.

The U2OS-ICP4/ICP27 -miR302GFP BAC construct carries in the LAT locus an expression cassette for the miR302s/367 cluster (Anokye-Danso, Cell Stem Cell, 8: 376-388 (2011)) as an alternative for the typical Yamanaka somatic cell reprogramming gene cocktail (OKSM: Oct4, Klf4, Sox2, c-Myc) (Takahashi and Yamanaka, Cell, 126: 663-676 (2006): Takahashi, Cell, 131: 861-872 (2007). The miR302s/367 gene cluster is located in an intron connecting the EF1α promoter to the GFP coding sequence. Two isolates of JΔNI7-miR302GFP BAC DNA were purified and introduced into U2OS-ICP4/ICP27 cells to produce virus particles for examination of virus growth and transgene expression. On observation of 90% cytopathic effect in the cultures, virus was collected from cells and supernatants and used to infect fresh U2OS-ICP4/ICP27 cells. Transgene expression and virus spread were then monitored daily. As shown in FIG. 3, at three days post infection, both BAC isolates produced plaque-forming virus and both viruses expressed EGFP and mCherry.

### EXAMPLE 6

This example describes the construction of a targeting plasmid for insertion of a tetracycline-inducible promoter and Gateway recombination cassette into the LAT locus of a HSV vector.

Different strategies may be used to prevent genetic rearrangement and inactivation of an OKSM expression cassette during virus expansion, where the cassette has been inserted into the LAT locus of JΔNI5 or JΔNI8. One of these strategies is to replace the constitutively active CAG promoter of the OKSM cassette with a tetracycline-inducible promoter. Because the tetracycline-inducible promoter is only active in the presence of both its transactivator (rtTA) and tetracycline/doxyxycline, transgene expression can be tightly regulated (repressed) during virus expansion.

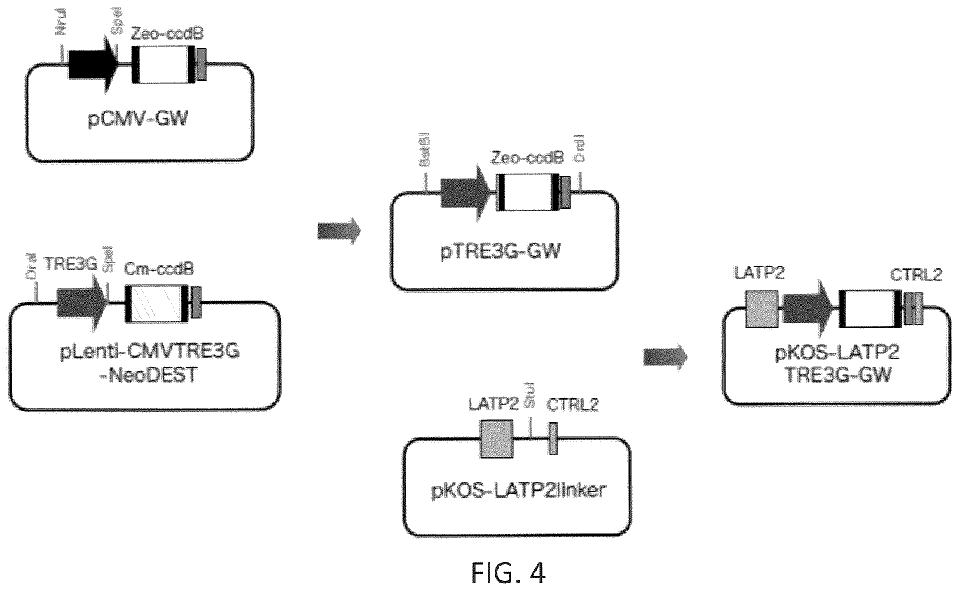

A targeting plasmid for insertion of the tetracycline-inducible promoter into the LAT locus was developed (FIG. 4). A lentivirus construct (pLenti-CMVTRE3G-NeoDEST, Addgene) carrying the tetracycline-inducible TRE3G promoter was used as a starting construct. The TRE3G promoter was isolated from pLenti-CMVTRE3G-NeoDEST as a Dral-Spel fragment and inserted between the Nrul and Spel sites of plasmid pCMV-GW, replacing the resident CMV promoter. Plasmid pCMV-GW contained a zeocin (Zeo)-selectable gene in the Gateway (GW) cassette instead of the chloramphenicol (Cm)-selectable gene in GW of the lentiviral plasmid. The TRE3G-GW (Zeo) expression cassette was isolated and cloned between LAT sequences in a plasmid containing a portion of the LAT locus to add “homology arms” to the TRE3G-GW (Zeo) cassette for recombination into BAC DNA.

### EXAMPLE 7

This example demonstrates immunofluorescence staining and a complementation assay for ICP27.

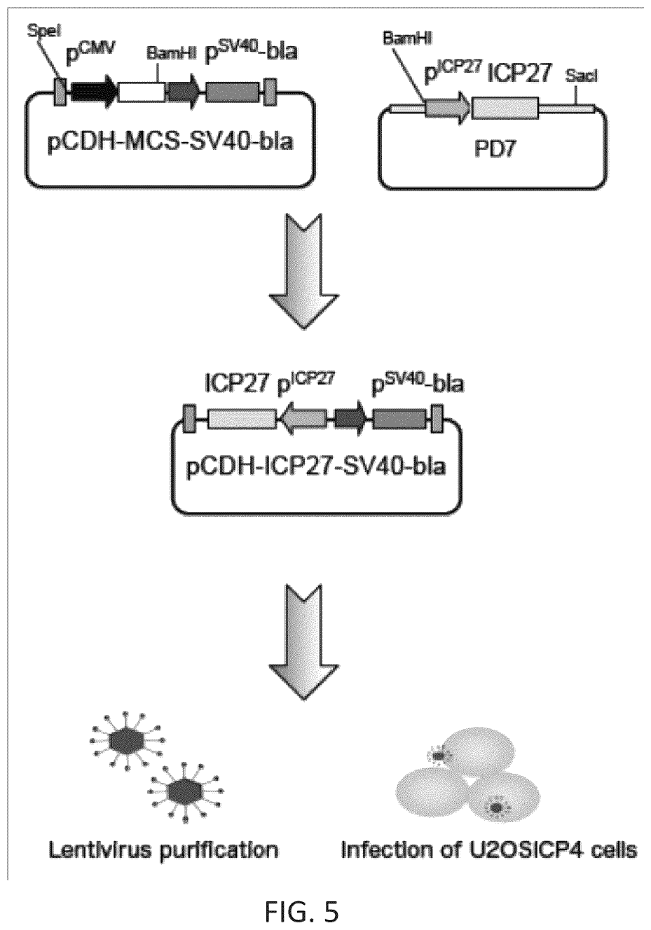

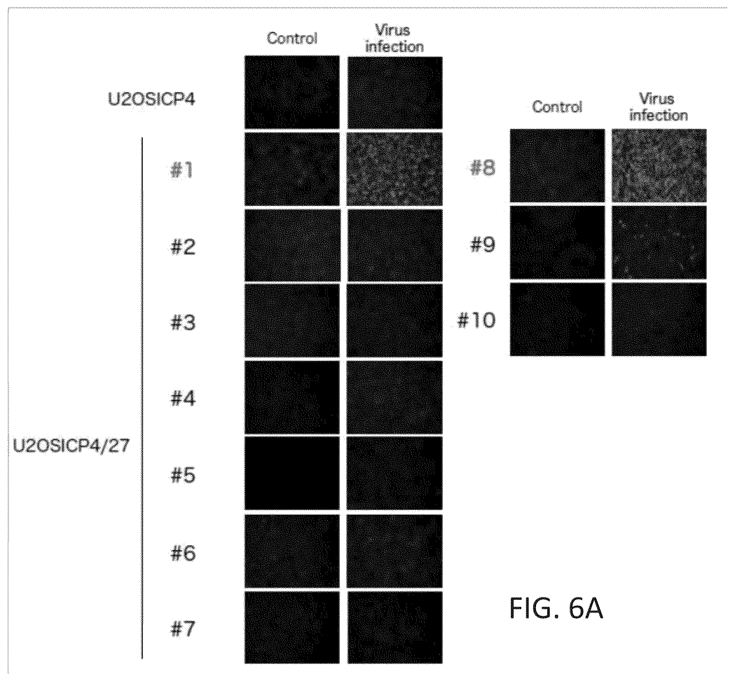

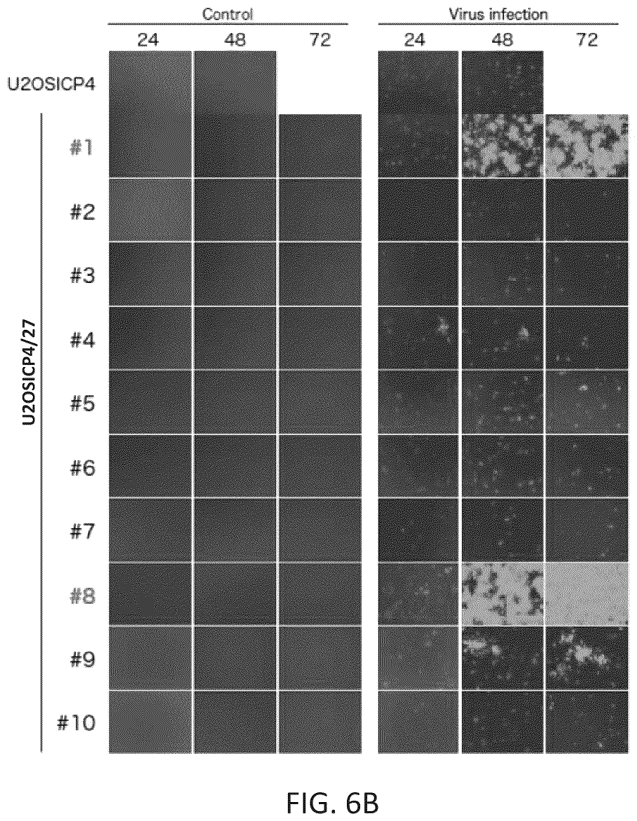

U2OS-ICP4 cells and individual clones of ICP27 lentivirus-infected U2OS-ICP4 cells selected for acquisition of resistance to blasticidin were infected with QOZHG virus (ΔICP4, ΔICP27::HCMV IEp-GFP, β-ICP22, β-ICP47, βUL41::ICP0placZ: Chen, J. Virol., 74: 10132-10141 (2000)) at MOI of 0.5 for ICP27 immunofluorescence staining and 0.01 for the complementation assay. For ICP27 immunofluorescence staining, the cells were fixed and stained 24 hours post-infection (FIG. 6A). Clones #1 and #8 showed robust induction of ICP27 expression by ICP27-deficient HSV (QOZHG) infection. For the complementation assay, virus growth was monitored, and photographs were taken at 24, 48, and 72 hours post-infection (FIG. 6B). U2OS-ICP4/27 clones #1 and #8 showed and the strongest ability to support QOZHG virus growth.

### EXAMPLE 8

This example demonstrates the construction and testing of several HSV vectors.

### Materials And Methods

### Cells

Human osteosarcoma U2OS cells (ATCC, Manassas, VA, USA) were grown in DMEM with 10% FBS and penicillin-streptomycin (P/S). Human neonatal dermal fibroblasts (HDFs) (ATCC, PCS-201-010) and BJ human foreskin fibroblasts (ATCC, CRL-2522) were grown in DMEM with 10% Embryonic Stem Cell Qualified FBS (Invitrogen) and P/S. Vero, Vero-7b (Krisky et al., Gene Ther. 5, 517-30 (1998)) and 293T cells were cultured in DMEM containing 5% FBS and P/S. Human hepatocytes (hHEP) were isolated and cultured as described (Ueki et al., Hepatology 54, 216-28 (2011): Yoshida et al., Hepatology 58, 163-75(2013)). Human subcutaneous preadipocytes (hPAD) (PT-5020, Lonza) were maintained with PBM™-2 Basal Medium (Lonza). Human muscle-derived stem/progenitor cells (hMDSCs) were cultured as described (Gao et al., Cell Transplant 22, 2393-408 (2013)). Human neonatal keratinocytes (hEK) (Invitrogen) were cultured in EpiLife®: Medium supplemented with Human Keratinocyte Growth Supplement (both from Invitrogen). Dorsal root ganglia (rDRGs) were micro-dissected from day 15 rat embryos, dissociated with 3 mg/ml type-I collagenase (Sigma, St. Louis, MO) in Leibovitz's L-15 media for 30 min at 37° C. with constant shaking, and plated on poly-D-lysine (Sigma)-coated coverslips at approximately 10⁵ cells/well in 24-well plates in 500 μl defined Neurobasal medium with B27 supplement, Glutamax-I, Albumax-II, and P/S (Gibco/Invitrogen, Grand Island, NY), supplemented with 100 ng/ml 7.0S NGF (Sigma). At 1-3 d post plating, cultures were treated with 10 μM uridine and 10 μM fluorodeoxyuridine (Sigma) in the above media for 1-2 d to limit the expansion of dividing cells such as fibroblasts and glia. Cells were then washed with PBS and incubated with NGF-supplemented Neurobasal medium as above. Virus infections were performed at 10-15 d after plating.

U2OS-ICP4 cells were generated by infection of U2OS cells with purified ICP4 lentivirus (see below), isolation of puromycin-resistant clones (2 mg/ml), and immunofluorescence screening for ICP4 expression after infection with ICP4-deficient virus (QOZHG) at a multiplicity (MOI) of 0.5. U2OS-ICP4/27 cells were similarly generated by infection of U2OS-ICP4 cells with purified ICP27 lentivirus, selection for resistance to puromycin and blasticidin (10 mg/ml), and screening of QOZHG-infected clones for ICP27 immunofluorescence and virus growth (FIG. 6).

### Lentiviruses

Lentiviral ICP4 expression plasmid pCDH-ICP4-puro was constructed by replacing the complete HCMV IE promoter of plasmid pCDH-CMV-MCS-EF1-Puro (Systembio) with the ICP4 promoter and coding region from plasmid S3 which consists of the SphI fragment from pICP4-lox-pac (Rasty et al., J. Neurovirol. 3. 247-64 (1997)) (GenBank JQ673480.1 HSV-1 KOS map positions 131,587-124,379; D. Krisky and J.C.G., unpublished) inserted in the SphI site of pUC19. Lentiviral ICP27 expression plasmid pCDH-ICP27-SV40-bla was constructed, first, by replacement of the puromycin-resistance cassette of pCDH-CMV-MCS-EF1-Puro with the blasticidin-resistance cassette of pcDNA6/BioEas™-DEST (Invitrogen) to create pCDH-CMV-MCS-SV40-bla (FIG. 5). The ICP27 promoter and coding region were then isolated by digestion of plasmid PD7 containing the ICP27 gene and flanking sequences between EcoRV and Sacl sites (GenBank JQ673480.1 map positions 110,580-115,666; D. Krisky and J.C.G., unpublished) with BamHI (map position 113,244) and SacI, and the isolated fragment was used to replace the CMV promoter in pCDH-CMV-MCS-SV40-bla. The resulting plasmid, pCDH-ICP27-SV40-bla, is depicted in FIG. 5.

Lentiviruses were produced using the ViraPower™ Lentiviral Packaging Mix (Invitrogen) according to the manufacturer's instructions. Briefly, 293T cells were transfected with pCDH-ICP4-puro or pCDH-ICP27-SV40-bla in ViraPowerTM mix. Supernatants were harvested 2 d later, clarified, filtered through a 0.45 μm filter, and concentrated by centrifugation. Lentiviruses produced from pCDH-ICP4-puro are referred to herein as ICP4 lentivirus: lentiviruses produced from pCDH-ICP27-SV40-bla are referred to as ICP27 lentivirus.

### HSV-BAC Engineering

All HSV-BAC constructs generated in this study and converted to virus particles are listed in Table 2 and were derived from KOS-37 BAC (24), a kind gift from D. Leib (Dartmouth Medical School, NH). All BAC engineering was performed by scarless Red recombination with pRed/ET (Gene Bridges, Heidelberg, Germany) and either pBAD-I-scel plasmid (kindly provided by N. Osterrieder, Free University of Berlin, Germany) or in E. coli strain GS1783 (from G. Smith, Northwestern University, Chicago, IL), as described (Tischer et al., Methods Mol. Biol. 634, 421-30 (2010): Tischer et al., Biotechniques. 40, 191-97 (2006)), or by in vitro Gateway (GW) recombination according to the Gateway Technology Manual (Invitrogen) (tools.lifetechnologies.com/content/sfs/manuals/gatewayman.pdf). All constructs were confirmed by PCR analysis, FIGE analysis of restriction enzyme digests, and targeted DNA sequencing. Targeting plasmids for Red recombination were constructed as described (Tischer et al., Biotechniques. 40, 191-97 (2006)). The kanamycin-resistance gene flanked by an I-SceI restriction site (I-SceI-aphAI fragment) was amplified from pEPkan-S2 (from N. Osterrieder) (Tischer et al., Biotechniques, 40, 191-97 (2006)) by PCR with the different targeting primers specified below and listed in Table 1. Primer portions targeting the viral genome were designed based on the sequence of HSV-1 strain-17 (GenBank JN555585.1). All targeting fragments for Red recombination were purified by Qiagen gel extraction kit (Qiagen) or SpinSmart Nucleic Acid Prep & Purification Columns (Denville Scientific). Nucleotide positions provided below refer to the GenBank JQ673480 sequence of HSV-1 strain KOS.

To construct the JΔNI vectors, the previously described hyperactive N/T double mutation was introduced into the gB gene (Uchida et al., J. Virol., 84, 12200-09 (2010)) of KOS-37 BAC and the internal repeat (joint) region was deleted. The I-SceI-aphAI fragment was cloned into the SnaBI site of plasmid pgB1:D285N/A549T (Uchida et al., J. Virol., 84, 12200-09 (2010)). The resulting plasmid, pgB: N/T-kan, was used as template for amplification with primers R21 and R22 (Table 1), and the product was recombined with the native gB gene of KOS-37 BAC followed by I-SceI-enhanced deletion of the aphAI gene in pBAD-I-sceI plasmid-transformed bacteria. Next, I-SceI-aphAI was amplified with nested forward primers 46, 48 and reverse primer 47 for Red-mediated deletion of the joint region (GenBank JQ673480 positions 117,080-132,466) along with the first 14 nucleotides (nts) of the adjacent unique short (Uₛ) segment of the genome, followed by removal of the aphAI gene. Uₛ in KOS-37 BAC is in the reverse orientation relative to the typical representation of the HSV genome, placing the Uₛ12 (ICP47) gene directly adjacent to the joint and the Uₛ1 gene adjacent to the Us terminal repeat. The 14-nt deletion beyond the joint (GenBank JQ673480 positions 145,377-145,390) thereby removed the ICP47 translation initiation codon.

Replacement of the ICP4 locus in the joint-deleted gB: N/T BAC with an mCherry expression cassette was achieved as follows. Plasmid pUbC-mCherry-SV40pA was constructed by cloning of the human ubiquitin C promoter (UbCp) from pBluescriptUB-Flag-mArt (a gift from H. Nakai, Oregon Health & Science University, Portland, OR), the mCherry gene from pEP-miR (Cell Biolabs, San Diego, CA), and the SV40 polyadenylation (polyA or pA) region from pEP4-EO2SCK2M-EN2L (Addgene plasmid 20924) (Yu et al., Science 324, 797-801 (2009)) into pBluescript KS+ (Stratagene). The I-Scel-aphAI fragment was then cloned into the BamHI site of pUbC-mCherry-SV40pA at the boundary between UbCp and mCherry to generate pUbC-mCherry-SV40pA-KAN. The insert was PCR amplified with primers 51 and 52 (Table 1) for Red-mediated recombination with the ICP4 target locus. The resulting construct was deleted for HSV-1 KOS positions 146,113-151,581 of the GenBank JQ673480 sequence, including the TAATGARAT motifs of the ICP22 promoter.

Replacement of the ICP0 and ICP27 IE promoters with the early (B) HSV-1thymidine kinase (TK) promoter to generate JΔNI2 was performed by PCR through the TK promoter in front of both the ICP0 and the ICP27 coding region in the JΔββ virus genome (Craft et al., Stem Cells 26, 3119-29 (2008)) with nested primer pairs 81/82 and 83/84, respectively, cloning of the product of each reaction into pCRblunt (Invitrogen) to produce pCRBlunt-β0) and pCRBlunt-B27, respectively, insertion of I-SceI-aphAI into the BglII site of pCRBlunt-β0) and the Hpal site of pCRBlunt-β27 yielding pCRBlunt-β0-KAN and pCRBlunt-β27-KAN, and PCR amplification of the inserts with primer pairs 85/86 and 87/88, respectively, for Red recombination with the joint-deleted UbCp-mCherry gB: N/T BAC. JΔNI3 was then derived from JΔNI2 by Red-mediated clean deletion of the ICP0 coding sequence using nested targeting forward primers 41. 42 and reverse primer 43 for I-SceI-aphAI amplification. JΔNI5 was likewise derived from JΔNI3 by clean deletion of the ICP27 coding sequence using primer pair 44/45 to produce the targeting I-SceI-aphAI fragment.

JΔNI6-CAGGFP was generated by insertion of an EGFP expression cassette between the UL3 and UL4 genes of JΔNI5. First. plasmid pCAG-GFP was constructed by replacing the gH gene between the CAG promoter (CMV enhancer/chicken beta-actin promoter/chimeric intron) and rabbit β-globin poly A region in plasmid pPEP100 (a gift from P. Spear, Northwestern University) (Pertel et al., Virology 279(1), 313-24 (2001)) with the EGFP gene from pEGFP-C1 (Clontech). I-SceI-aphAI was then inserted into the SnaBI site of pCAG-GFP to create plasmid pCAG-GFPKAN. Separately. multi-step PCR using KOS-37 BAC DNA as initial template was performed to generate a fragment that contained novel cloning sites (MCS) between the UL3 and UL4 poly A regions, as follows. First, extension PCR was performed to amplify the 3′ UTRs of UL3 and UL4 with primer pairs 57/58 and 59/60, respectively. that added an overlapping MCS region to each 3′UTR fragment. The 2 PCR products were gel-purified and 100 ng of each was used for overlapping PCR with primers 57 and 59 to create a continuous fragment. This product was cloned into pCRBlunt (Invitrogen). yielding plasmid pCRBluntUL3-4linker. The insert of pCAG-GFPKAN was then cloned between the AccI and PsiI sites in the MCS of pCRBluntUL3-4linker. The resulting plasmid was digested with MfeI and PpuMI and the UL3-CAG-GFPKAN-UL4 fragment was isolated for recombination with the UL3-UL4 intergenic region of JΔNI5 followed by aphAl gene removal.

To create JΔNI7GFP. an Xhol fragment (˜6.2 kb) containing the 2 CTRLs. LAP1 and LATP2 of the HSV-1 LAT locus was isolated from KOS-37 BAC DNA and cloned into pBluescript KS+. An internal KpnI-SalI fragment extending from near the end of LATP2 to ˜250) bp downstream of CTRL2 was isolated from this recombinant and cloned between the KpnI and SalI sites of pSP72 (Promega) to produce pSP72KOS-LAT. A multicloning site was then introduced between 2 BstXI sites located ˜240 and ˜430 bp downstream of the KpnI site. yielding pSP72KOS-LATlinker. Separately. plasmid pCAG-GW was constructed by replacing the gH gene of pPEP100 (Pertel et al., Virology 279(1). 313-24 (2001)) with a PCR-amplified modified GW recombination cassette [GW-Zeo; zeocin resistance instead of chloramphenicol resistance (Wolfe et al., J. Virol. 84, 7360-68 (2010))]. The insert of pCAG-GW was then cloned into the MCS of pSP72KOS-LATlinker to produce pSP72KOS-LATlinker-GW. The plasmid was digested with KpnI and HpaI to isolate the CAG-GW region with flanking LAT sequences for Red-mediated recombination with the LAT locus of JΔNI5 in cedB-resistant HerpesHogs bacteria (Wolfe et al., J. Virol. 84, 7360-68 (2010)). Finally. the GW cassette in the resulting JΔNI5 recombinant BAC was replaced with the EGFP gene by Red-mediated recombination with an AatII-PsiI fragment, including CAG and poly A sequences, of plasmid pCAG-GFPKAN. JΔNI77GFP derivatives deleted for spedific LAT region elements outside the EGFP cassette (CTRL1, CTRL2, LATP2) were generated by Red-mediated recombination of JΔNI7GFP DNA with targeted I-SceI-aphAI cassettes produced by PCR with the respective F1, F2 and R primers listed in Table 1 (63-65, 66 -68, and 69-71, respectively).

For the construction of the different JΔNI19 and JΔNI10 vectors, the ˜6.2-kb LAT XhoI fragment described above was deleted from the JΔNI5 genome by recombination with LAT-targeted I-SceI-aphAI generated by PCR with primers 78, 79 and 80, producing JΔNI5ΔL. GW-Zeo was then amplified with targeting primers for the intergenic region between UL45 and UL46 (74/75) or UL50 and UL51 (76/77) and the product of each reaction was recombined with JΔNI5ΔL BAC DNA to create JΔNI9GW and JΔNI10GW. JΔNI7 GFP BAC DNA was digested with Xhol and the ˜7.2-kb CAG-GFP-containing fragment from the LAT locus was isolated and cloned into pENTRIA (pENTR-LAT-XhoI). The corresponding ˜5.3-kb Xhol fragment of JΔNI7GFPΔC12LP2 was likewise isolated and cloned into pENTRIA (pENTR-LATA-Xhol) and lastly, the insert of pCAG-GFP (see above) was transferred into pENTRIA (pENTR-CAG-GFP). In vitro L R Clonase (Invitrogen) reactions were then performed to recombine the different pENTR constucts with JΔNI10GW BAC DNA, producing JΔNI10LAT-GFP, JΔNI10 C12LP2-GFP and JΔNI10OGFP, respectively, and pENTR-LAT-XhoI and pENTR-CAG-GFP were recombined with JΔNI9GW BAC DNA to generate JΔNI9LAT-GFP and JΔNI9GFP.

KNTc was constructed by introduction of the gB: N/T mutations into KOS-37 BAC as described above and the UbCp-mCherry cassette into the intergenic region between UL3 and UL4 locus of KOS-BAC by Red recombination with I-SceI-aphAI targeted by amplification with primers 53 and 54.

### Viruses

JΔNI BAC DNAs were converted to infectious viruses by transfection of U2OS-based complementing cells. DNA in 500 μl OptiMEM (Invitrogen) was incubated with 1 μl Lipofectamine Plus Reagent (Invitrogen) for 5 min at room temperature, 6.25 μl Lipofectamine LTX (Invitrogen) was added, the mixture was incubated for 30 min at room temperature and added to cells. After incubation at 37° C. for 6 h, the transfection mix was removed and the cells were cultured overnight at 37° C. with serum-free DMEM, transferred to a 33° C. incubator, and monitored for 100% cytopathic effect (CPE). Supernatants were titered and then amplified by infection of sequentially larger cultures at a multiplicity (MOI) of 0.001 PFU/cell. KNTc infectious virus was produced by transfection of Vero cells using 3 μl Lipofectamine Plus Reagent and 9 μl Lipofectamine LTX for 4 h at 37° C.; an MOI of 0.01 PFU/cell was used for KNTc virus amplification on Vero cells. Complementing cells for transfections and/or virus growth were as follows: U2OS-ICP4 (JΔNI2, JΔNI3), U2OS-ICP4/27 (JΔNI5 and derivatives), and Vero-7b [QOZHG virus (AICP4, ΔICP27::HCMV IEp-GFP β-ICP22, β-ICP47 and ΔUL41::ICP0p-lacZ) (Chen et al., J. Virol. 74, 10132-41 (2000))]. All virus stocks were titered on U2OS-ICP4/27 cells (Table 2). Physical titers [genome copies (gc)/ml] were determined by quantitative real-time PCR as described below. Fluorescent images of infected cells were obtained with a Nikon Diaphot fluorescence microscope (Nikon, Melville, PA) at 40× magnification.

### Virus Growth Curves

Replicate wells of Vero-7b, U2OS, U2OS-ICP4 and U2OS-ICP4/27 cells in a 24-well plate were infected at a multiplicity (MOI) of 0.001 for 2 h, treated with 0.1 M glycine (pH 3.0) for 1 min to inactivate extracellular virus, and incubated at 37° C. and 5% CO₂. Media were harvested daily and titered by standard plaque assay on U2OS-ICP4/27 cells.

### Cytotoxicity Assay

5×10³ HDF and Vero cells were seeded in a 96-well plate and infected with KOS, QOZHG or JΔNI viruses at 25,000 gc/cell. Cell viability was determined 5 d later by MTT assay essentially as described (Uchida et al., J. Virol. 87, 1430-42 (2013)).

### Western Blotting and Immunofluorescence

Cell lysate preparation and Western blotting were performed as described (Miyagawa et al., PLoS One 4, e4634 (2009)). Polyclonal rabbit anti-ICP0 antibodies were produced in our laboratory, anti-ICP27 (10-H44) was from Fitzgerald Industries International (Concord, MA), anti-ICP22 was a gift from John Blaho (Mt Sinai School of Medicine, NY), anti-ICP4 (10F1) was from Santa Cruz Biotechnology, and anti-α-tubulin (T6793) was from Sigma. Immunofluorescence using the sameJ. Virol. 83, 2951-61 (2009)) and was examined under a Nikon Fluorescence microscope.

Quantitative Reverse Transcription-PCR (qRT-PCR) and Genome PCR

For qRT-PCR, total RNA was typically extracted by RNeasy kit (Qiagen). cDNA was synthesized with a RETROscriptR Kit (Ambion). Real-time PCR was carried out by the StepOnePlus Real-Time PCR System (Applied Biosystems). For small numbers of cells, a Cells-to-cDNA™ II Kit (Ambion) was used for cell lysis and reverse transcription. Results for 18S ribosomal (r) RNA were used to normalize the data. All qRT-PCR primers used in this study are listed in Table 1.

For the determination of physical (genome copy) titers of virus stocks, 5 l of virus was incubated with 300 U/ml of Benzonase nuclease (Sigma) for 1 h at 25° C. in the presence of 2 mM MgCl₂ and viral DNA was extracted by DNeasy Blood & Tissue Kit (Qiagen). gc titers were determined by qPCR for the glycoprotein D (gD) gene with the gD primers (38/39) and probe (40) listed in Table 1. Amounts of nuclear viral DNA were determined by rinsing the cells at 2 hpi, isolation of nuclei as described (Dignam et al., Nucleic Acids Res. 11, 1475-89 (1983): Suzuki et al., BMC Res. Notes 3, 294 (2010)), DNA extraction with the DNeasy Blood & Tissue Kit, and qPCR for the gD gene as above. Cellular 18S ribosomal DNA levels were measured with the use of TaqMan® Ribosomal RNA Control Reagents (Invitrogen) and were used to normalize viral DNA amounts.

### Statistics

All values are presented as the mean+/−SD. Differences between pairs were analyzed by Student's/test using Microsoft Excel 14.4.1. P values below 0.05 (P<0.05) were considered statistically significant.

### Results

### Vector Engineering and Virus Growth

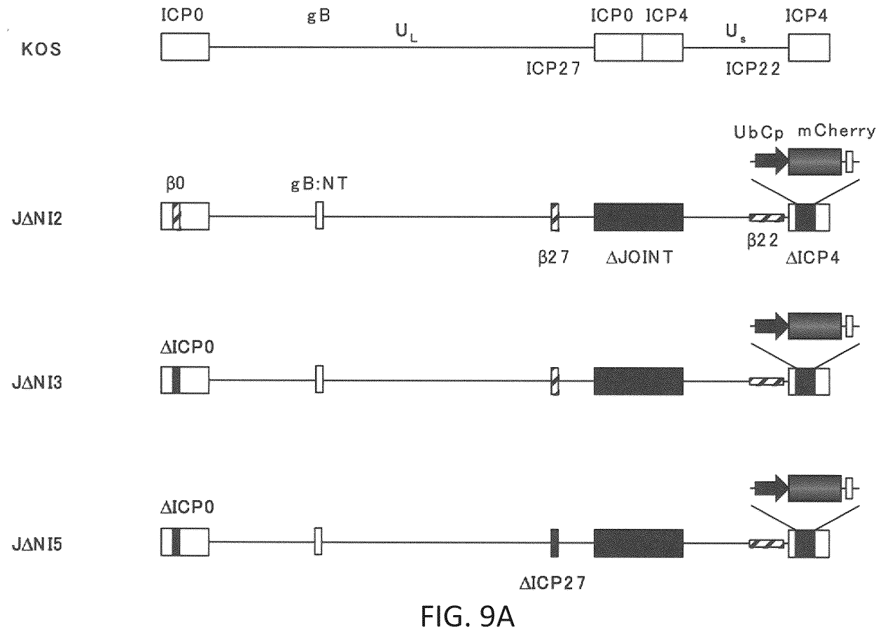

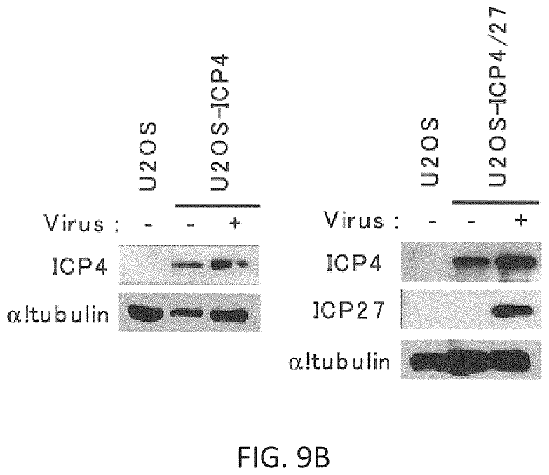

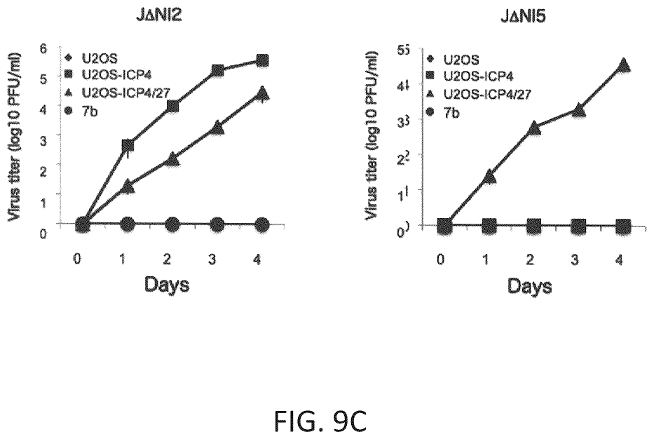

Efficient transduction of mouse embryonic stem cells (mESC) with a defective HSV vector, JDββ, has been reported (Craft et al., Stem Cells 26, 3119-25 (2008)), without detectable damage to the cells. JDBB was deleted for the internal repeat region (“joint”) of the HSV genome and for two IE genes (ICP4 and ICP22). In addition, the promoters of two other IE genes (ICP0 and ICP27) were replaced with copies of the viral thymidine kinase (TK) early (E or β) gene promoter. JDββ-infected non-complementing cells showed minimal IE gene expression, but the vector was capable of efficient transgene expression in mESC without interference with embryoid body formation or developmental transcription programs. To facilitate the utilization of this vector backbone for diverse gene transfer applications, Red-mediated recombineering was performed in bacteria (Tischer et al., Biotechniques 40, 191-97 (2006)) to derive backbone. JΔNI2 (FIG. 9A), from KOS-37 BAC, a bacterial artificial chromosome (BAC) containing the complete genome of HSV-1 strain KOS (Gierasch et al., Virol. Methods 135:197-206 (2006)). JΔNI2 was deleted for ICP4 and the joint, including the ICP47 promoter. contained the same ICP0 and ICP27 promoter replacements as JDββ, and the consensus VP16-binding (TAATGARAT) motifs in the ICP22 regulatory region were deleted to change the kinetics of ICP22 expression to that of an early gene. To visualize infection and monitor viral transcriptional activity. an mCherry reporter gene expression cassette was introduced at the position of the deleted ICP4 locus. In addition. the glycoprotein B gene was replaced with a hyper-active allele, gB:N/T (Uchida et al., J. Virol 84: 12200-9 (2010)). to enhance virus entry into the cells. To eliminate the potential for low-level production of the toxic ICP0 and ICP27 proteins in non-complementing cells, two derivatives of JΔNI2 also were constructed by deletion of the complete coding sequences for ICP0 (JΔNI3 ) or ICP0 and ICP27 (JΔNI5 ) (FIG. 9A).

To convert the different JΔNI vector constructs to infectious virus particles. a cell line capable of complementing ICP0. ICP4 and ICP27 was created. Human osteosarcoma U2OS cells naturally complement ICP0 (Yao et al., J. Virol. 69, 6249-58 (1995)), eliminating the need to express this toxic protein. U2OS cells were transduced with a lentivirus carrying the ICP4 gene under the control of its own regulatory sequences and a clonal line was isolated that permanently expresses ICP4 (U2OS-ICP4: FIG. 9B). Transduction of these cells with a second lentivirus carrying the ICP27 gene and control region (FIG. 6) was then used to select a cell line. U2OS-ICP4/27. that in addition expresses ICP27 on infection with an ICP4/ICP27-deleted virus (FIG. 9B and FIG. 6). While the abilities of U2OS cells to activate the ICP4 promoter in the absence of the viral VP16 protein and tolerate sustained ICP4 expression were unanticipated. it is unknown whether these features relate to the native ICP0-complementing activity of these cells. DNA from BAC constructs JΔNI2 and JΔNI3 could be converted to infectious virus by transfection of U2OS-ICP4 cells, while DNA from the JΔNI5 construct yielded infectious particles only when transfected into U2OS-ICP4/27 cells (data not shown). FIG. 9C illustrates the growth of ΔNI2 and JΔNI5 viruses initially produced by transfection of U2OS-ICP4 or U2OS-ICP4/27 cells with JΔNI2 or JΔNI5 BAC DNA, respectively. Neither virus could grow on unmodified U2OS cells or on Vero-7b cells that complement ICP4 and ICP27, but not ICP0. JΔNI2 was able to grow on U2OS-ICP4 cells without ICP27 complementation whereas JΔNI5 growth required this additional complementing activity. The stability of the JΔNI5-complementing properties of the U2OS-cells was tested by plaque assay at different cell passages and no significant decline was observed in plaquing efficiency through at least 20 passages (Table 3). These results were consistent with the engineered IE gene modifications in these viruses and allowed for comparisons of their biological properties.

### Standardization of Virus Input

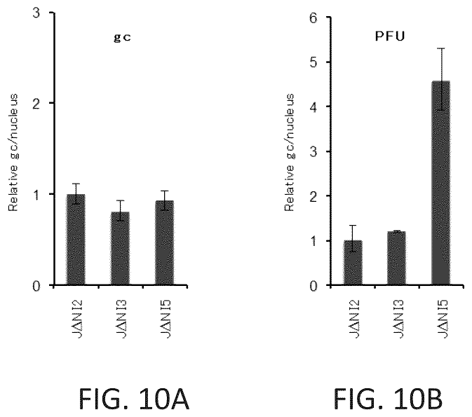

The relative amounts of each virus needed to deliver equal amounts of viral DNA to the infected cell nucleus was assessed. The biological and physical titers of the various virus stocks were determined by standard plaquing assay on U2OS-ICP4/27 cells and qPCR for the viral glycoprotein D gene, respectively: a difference in genome copy (gc) to plaque-forming units (PFU) ratios of approximately 3-fold was observed between the JΔNI2, JΔNI3 and JΔNI5 virus stocks (Table 2). HDFs were infected with equal PFU or equal gc of the 3 viruses and the number of HSV genomes in the cell nuclei at 2 hours (h) post infection (pi) was determined by qPCR. When equal gc were used for infection, the number of viral genomes in the nuclei at 2 hpi were similar between JΔNI2-, JΔNI3-and JΔNI5-infected cells (FIG. 10A). In contrast, infection with equal PFU resulted in a higher number of JΔNI5 genomes in the nuclei than JΔNI2 or JΔNI3 genomes (FIG. 10B). Thus in the remainder of this study, gc numbers were used to standardize virus input.

### Characterization of Vector Properties in Non-Complementing Cells

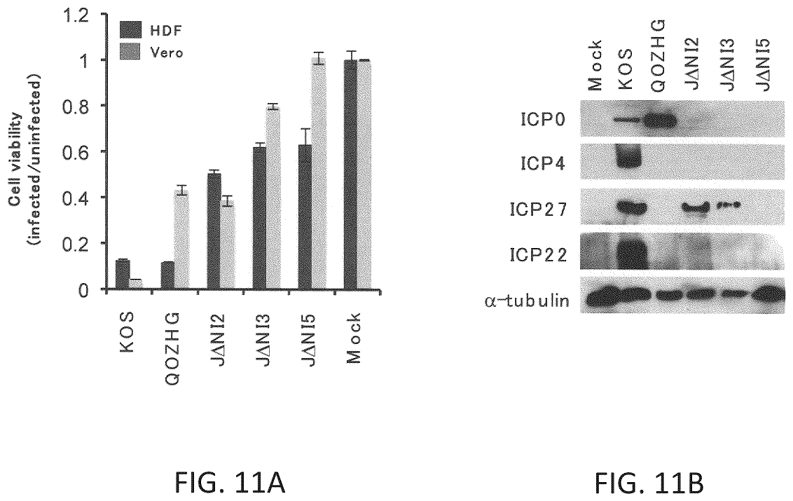

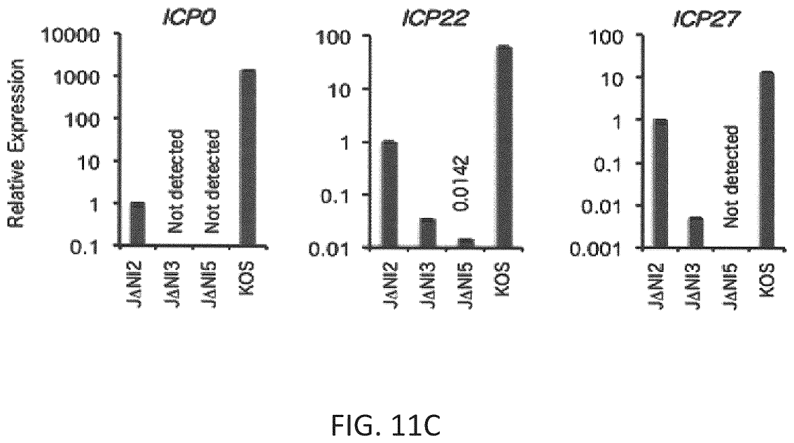

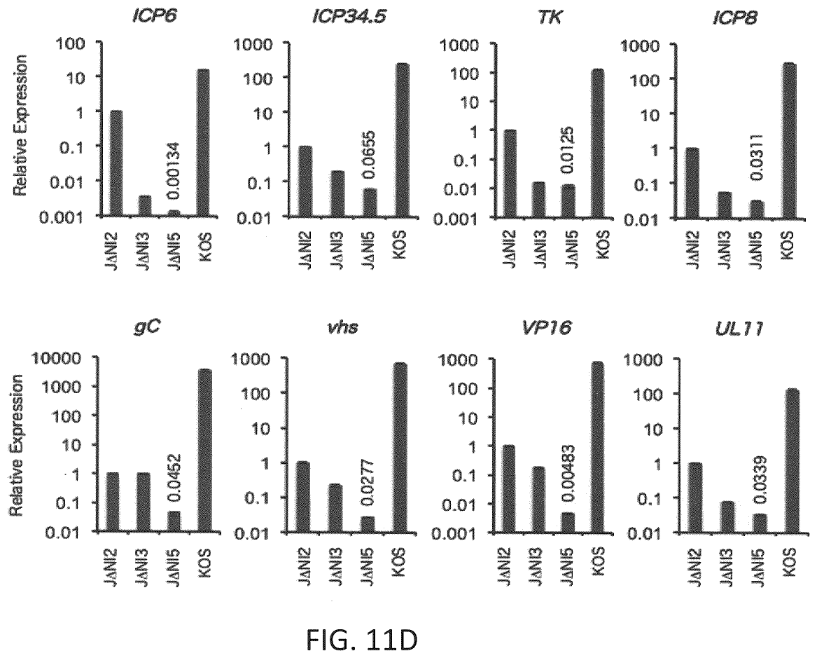

The effects of JΔNI2, JΔNI3 and JΔNI5 infection on the viability of non-complementing HDFs and Vero cells compared with wild-type KOS virus and a previous IE gene-deleted vector, QOZHG (ICP4/ICP27/β-ICP22/β-ICP47; (Chen et al., J. Virol. 74, 10132-41 (2000)) was first examined. At 5 days (d) pi with 25,000 gc/cell, approximately 4-5 times more viable cells remained in the JΔNI-infected HDF cultures than in the KOS- or QOZHG-infected cultures while smaller differences were seen between JΔNI-and QOZHG-infected Vero cells (FIG. 11A). Among the JΔNI viruses, JΔNI2 was somewhat more toxic to HDFs than JΔNI3 (p=0.0017) and 5 (p=0.0430) while toxicity for Vero cells decreased from JΔNI2 to JΔNI3 (p<0.001) to JΔNI5 (p<0.001). To correlate these findings with IE gene expression, Western blots of infected HDFs at 24 hpi were probed with antibodies to 4 of the 5 IE proteins (FIG. 11B). JΔNI2 and JΔNI3 both showed residual ICP27 expression, indicating that the TK promoter in front of the ICP27 gene in both vectors is not silent in these cells, but no IE proteins were detected in JANISJΔNI5-infected cells despite the use of 2-3 fold more gc/cell (equal PFU/cell, see Table 2). The KOS and QOZHG patterns were consistent with the reported down-regulation of ICPO expression by ICP4 (Douville et al., Virology 207, 107-16 (1995): Resnick et al., J. Virol. 63, 2497-503 (1989)). Using quantitative reverse transcription-PCR (qRT-PCR) analysis, ICPO mRNA was detected in JΔNI2-infected HDFs and decreasing levels of ICP22 and ICP27 mRNAs between JΔNI2-, JΔNI3-and JΔNI5-infected cells at 12 hpi with equal gc/cell (FIG. 11C). These results suggested that JΔNI2 produced sufficient levels of ICP0 to enhance ICP22 and ICP27 expression and confirmed that progressive deletion of IE genes reduces HSV vector cytotoxicity (Krisky et al., Gene Ther. 5, 1593-603 (1998): Samaniego et al., J. Virol. 72, 3307-20 (1998): Samaniego et al., J. Virol. 71, 4614-25 (1997)).

qRT-PCR was used to examine the expression of selected early and late viral genes (FIG. 11D). At 12 hpi, JΔNI2 generally expressed the highest levels of these genes and JΔNI5 the lowest, similar to the pattern observed for the IE genes. These results showed that substitution of the native ICP0 and ICP27 promoters with an early promoter was not sufficient to silence the viral genome in the absence of ICP4 while deletion of both genes drastically reduced residual gene expression.

### JΔANI Reporter Gene Expression

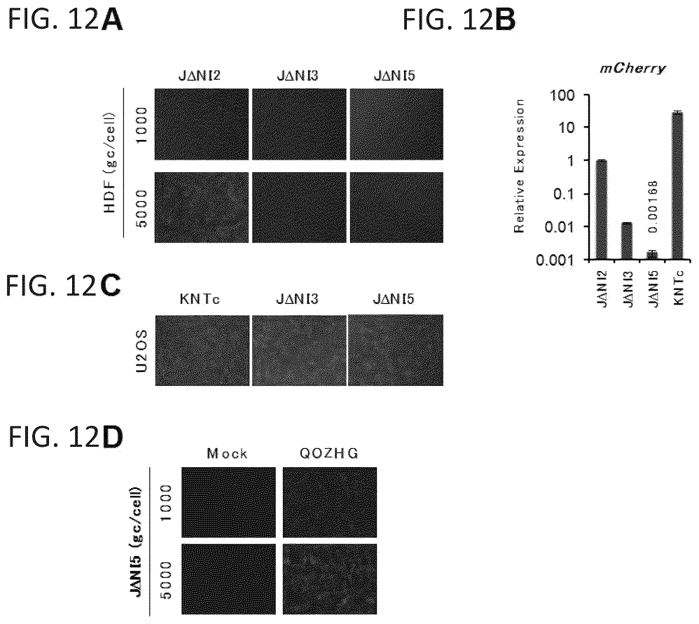

To determine whether the activity of the exogenous human ubiquitin C (UbC) promoter (p) introduced with the mCherry reporter gene into the deleted ICP4 locus of the JΔNI vectors was also down-regulated in JΔNI3-and JΔNI5-infected cells compared to JΔNI2-infected cells, mCherry expression in infected HDFs was examined. At 24 hpi with 5,000 gc/cell, mCherry fluorescence was readily detectable in JΔNI2-infected cells but not in JΔNI3-or JΔNI5-infected cells (FIG. 12A). Measurement of mCherry mRNA levels at 6 hpi by qRT-PCR revealed a reduction in transcriptional activity from the UbC promoter of some 100-fold between JΔNI2-and JΔNI3-infected cells, and an additional reduction of approximately 5-fold in JΔNI5-infected cells (FIG. 12B): the KNTc control virus used in this experiment was replication-competent and contained the UbCp-mCherry cassette in the intergenic region between UL3 and UL4. These data were consistent with the interpretation that the residual ICP0 and ICP27 expression from JΔNI2 (FIG. 11C) is sufficient to prevent global transcriptional silencing of the viral genome while deletion of these two genes essentially eliminates all native (FIG. 11D) as well as exogenous (FIG. 12A, 12B) promoter activity. In contrast to HDFs, standard U2OS cells infected with JΔNI3 or JΔNI5 S showed strong mCherry expression (FIG. 12C), suggesting that the ICPO-complementing activity of U2OS cells was sufficient to activate these otherwise silent genomes. To confirm that the viral ICP0 protein possessed the same activity. JΔNI5S-infected HDFs were superinfected with the ICP0+ QOZHG virus (see FIG. 11B). As illustrated in FIG. 12D. super-infection induced mCherry expression. while mock super-infection did not. Together. these results demonstrated that ICP27 was not required to derepress the silent JΔNI5 genome, at least in the presence of abundant ICP0 expression or complementing activity. and suggested that the minimal amount of ICP0 expressed in JΔNI2-infected HDFs sufficed to maintain limited transcriptional activity throughout the viral genome.

High-Level Transgene Expression from the Silent JΔNI5 Genome

Upon infection of neuronal cells. HSV enters a latent state in which the viral genome is transcriptionally silent except for the LAT locus. Whether the LAT locus would similarly remain active in non-neuronal cells in the context of an otherwise silent viral genome was investigated. To this end. an expression cassette consisting of the CAG enhancer/promoter and EGFP gene (CAGp-GFP) was introduced into the LAT 2-kb intron region of JΔNI5. creating a vector construct referred to as JΔNI7GFP (FIG. 13A). As a control. the same CAGp-GFP cassette was introduced into the UL3-UL4 intergenic region of JΔNI5. producing vector JΔNI6GFP (FIG. 13A): the UL3-UL4 intergenic region has been used for non-disruptive insertion of transgene cassettes (Baines et al., J. Virol. 65, 938-44(1991); Menotti et al., J. Virol. 76, 5463-71 (2002)) and is close to but outside the LAT locus. Elsewhere herein. “JΔNI6GFP” is referred to as “JΔNI6-CAGGFP” (See also FIG. 1A). Infectious viruses were produced on U2OS-ICP4/27 cells and the gc: PFU ratios of the new virus stocks were similar to that of JΔNI5 (Table 2). Both JΔNI6-CAGGFP and JΔNI7GFP produced abundant green and red fluorescence during amplification in U2OS-ICP4/27 cells. confirming the integrity of their transgene expression cassettes. To examine the abilities of these viruses to express the EGFP transgene in non-complementing cells. HDFs were infected with increasing gc/cell and EGFP fluorescence was recorded at 3 dpi (FIG. 13B). JΔNI7GFP-infected cells showed abundant. viral dose-dependent EGFP expression. whereas JΔNI6-CAGGFP infection produced limited expression even at the highest dose. Although a higher virus input was used here than in earlier JΔNI5 infections, mCherry expression remained minimal. These results were confirmed by qRT-PCR measurements of EGFP and mCherry mRNA levels at 3 and 5 dpi (FIG. 13C) and were consistent with the suggestion that the JΔNI6-CAGGFP and JΔNI7GFP virus genomes, like the JΔNI5 genome, were silent in infected HDFs while the LAT locus remained transcriptionally active. Since EGFP mRNA levels in JΔNI7GFP-infected cells were at least as high at 5 dpi as at 3 dpi (FIG. 13C), whether expression could be detected at later times when the cells are fully contact-inhibited was investigated. The results showed that expression could persist for at least 4 weeks in some of the cells (FIG. 13D). Together, these observations indicated that the LAT locus is a privileged site for durable transgene expression from an HSV genome that is non-toxic due to functional deletion of all IE gene expression.

CTRLs and LATP2 Support Transgene Expression from the LAT Locus

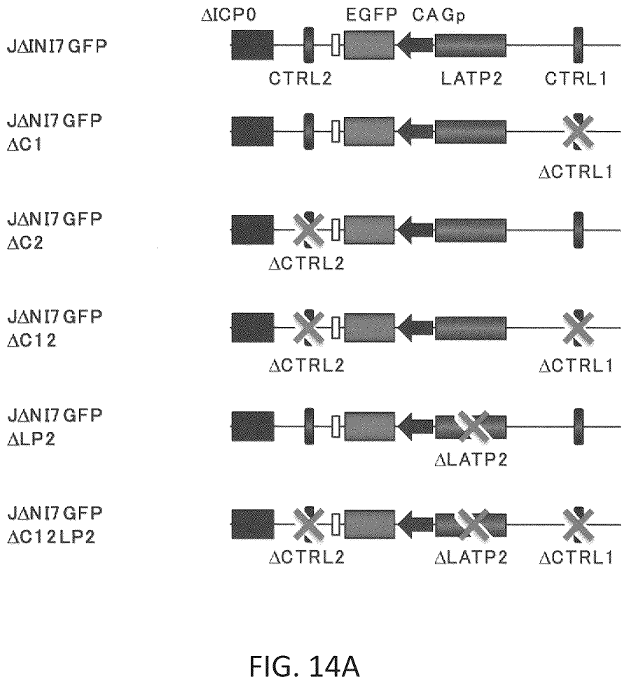

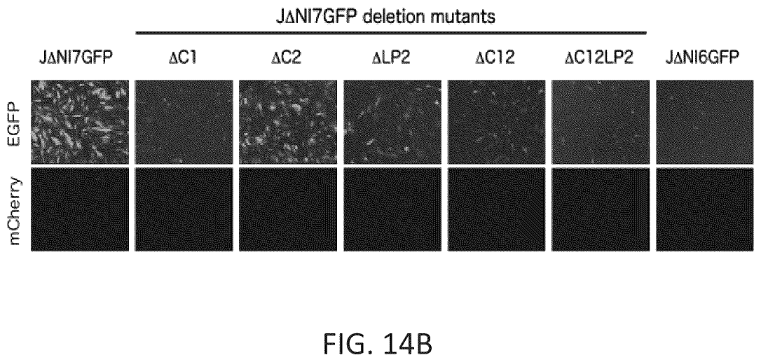

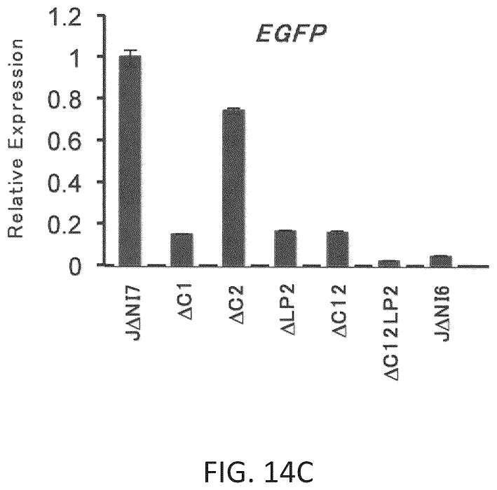

It has been demonstrated that LAT expression during latency is controlled by the latency-associated promoters (LAPs) 1 and 2 (Goins et al., J. Virol. 68, 2239-52 (1994); Zwaagstra et al., Virology 182, 287-97 (1991)). LAP1 is located upstream of the transcription initiation site of the ˜8.7-kb unstable primary LAT transcript that is processed to a stable 2-kb LAT intron, while LAP2 is located downstream of LAP1 in the first exon and extending into the 2-kb intron region. A LAP2-containing region extending somewhat further into the intron has been referred to as LATP2. In addition to acting as a promoter, it has been shown that the LAP2/LATP2 region can act as a position-independent long-term expression/enhancer element for transgene expression in neurons (Berthomme et al., J. Virol. 74, 3613-22 (2000); Palmer et al., J. Virol. 74, 5604-18 (2000)). However, it has also been reported that the LAT locus is protected from global silencing of the viral genome during latency by regions rich in CTCF binding sites, termed CTRLs, one located upstream of LAP1 (CTRL1) and the other in the 2-kb intron just downstream of LATP2 (CTRL2) (Blooom et al., Biochim. Biophys. Acta 1799, 246-56 (2010)). Thus, the potential roles of the LATP2 and CTRL elements in enabling the observed EGFP expression in JΔNI7GFP-infected HDFs was explored. The three elements were deleted separately and in combinations from the JΔNI7GFP genome (FIG. 14A) and reporter gene expression in infected HDFs was examined (FIGS. 14B, 14C). Deletion of either CTRL1 (ΔC1) or LATP2 (ΔLP2) caused a marked decrease in green fluorescence and EGFP mRNA levels whereas the deletion of CTRL2 (ΔC2) had only a minor effect. Deletion of both CTRLs (ΔC12) had the same effect as the deletion of CTRL1 alone while deletion of all three elements (ΔC12LP2) reduced expression further to the level observed in JΔNI6-CAGGFP-infected cells. No mCherry fluorescence was detectable in any of the infected cells (FIG. 14B), suggesting that the different deletions did not cause derepression of other sites in the viral genome. These results indicated that both CTRL1 and the LATP2 region play significant roles in protecting the linked transgene from transcriptional silencing in the context of a viral genome that is functionally devoid of all IE genes.

### LAT Locus Protection of Transgene Expression is Position-Independent

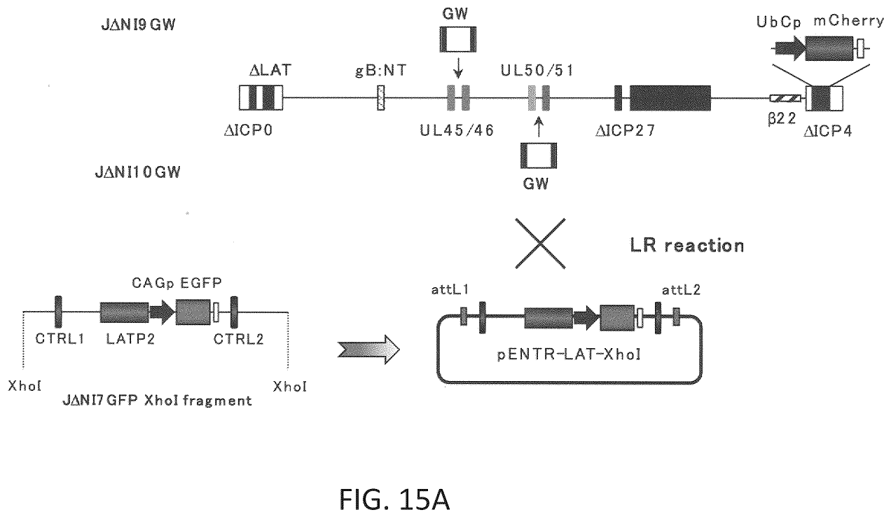

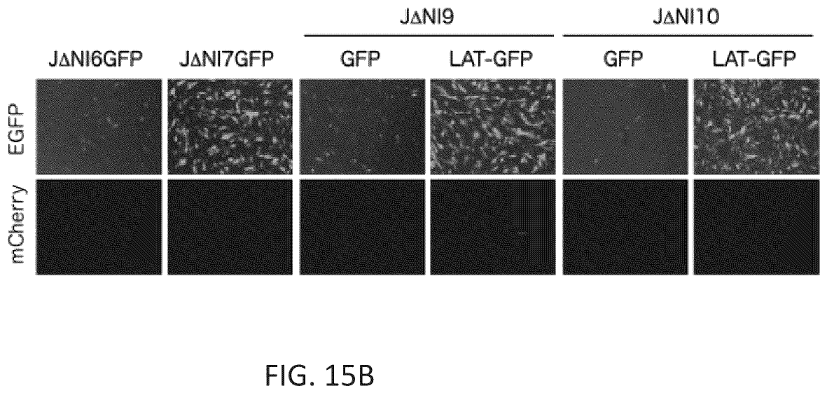

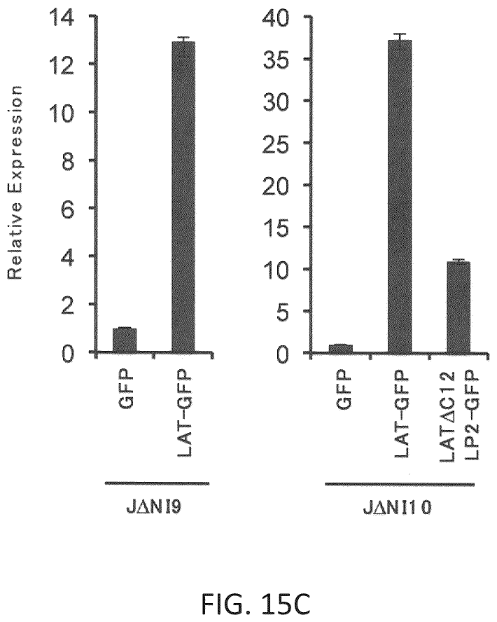

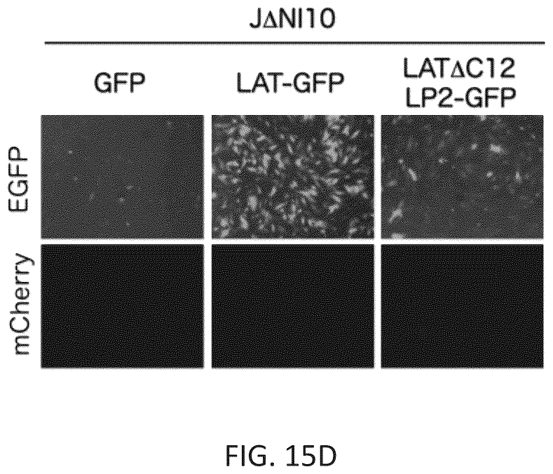

To determine whether sequences associated with the LAT locus are sufficient to protect an embedded transgene expression cassette against silencing in the absence of IE gene products, a restriction fragment corresponding to the LAT: CAGp-GFP region of JΔNI7GFP, including the two CTRLs, LAP1 and LATP2, was inserted at one of two ectopic positions in a JΔNI5 derivative that was deleted for the same LAT region to avoid recombination between the native and new sites. First, a Gateway (GW) recombination cassette was introduced into the intergenic region between UL45 and 46 (JΔNI9GW) or between UL50 and UL51 (JΔNI10GW) of the LAT-deleted JΔNI5 genome and then the LAT: CAGp-GFP fragment or just CAGp-GFP without LAT sequences were introduced by recombination with the GW cassette (FIG. 15A). Following virus production on U2OS-ICP4/27 cells (Table 2), the vectors were tested for reporter gene expression in infected HDFs. At 3 dpi, the vectors that lacked LAT sequences surrounding the reporter cassette (JΔNI9GFP and JJΔNI10GFP) showed low levels of EGFP fluorescence (FIG. 15B) and mRNA (FIG. 15C) in infected cells, similar to the JΔNI6-CAGGFP control vector, suggesting that the two intergenic insertion sites were transcriptionally repressed, like the intergenic region between UL3 and UL4 (FIG. 13). However, when the reporter cassette was flanked at both sides by LAT sequences (vectors JΔNI9LAT-GFP and JΔNI10LAT-GFP), EGFP expression increased to the level observed in JΔNI7GFP-infected cells (FIG. 15B), indicating that the anti-silencing activity of the LAT-derive regions is functional in a position-independent manner. To confirm the dependence of this activity on LATP2 and either or both CTRLs, a JΔNI2-and CTRL-deleted version of the LAT: CAGp-GFP fragment was introduced by GW recombination into JΔNI11GW genome: the deletions were the same as those in the LAT region of the earlier JΔNI7GFPΔC12LP2 vector (FIG. 14A). The deletions reduced EGFP expression in infected HDFs, although not fully down to the level observed with the LAT-free JΔNI10GFP vector (FIG. 15C, 15D): it is unclear why the ΔC12LP2 deletions appear to have a smaller effect here (˜3.5-fold) than in JΔNI7GFP (˜50-fold, FIG. 14C). However, these results clearly demonstrate that a portion of the LAT locus that includes the 2 CTRLs. LAP1 and LATP2 can protect an embedded transgene expression cassette in a position independent-manner against global silencing of the viral genome in the absence of IE gene expression and indicate that at least CTRL1 and LATP2 play a role in this activity.

### LAT Locus Elements Protect Transgene Expression in Other Cell Types

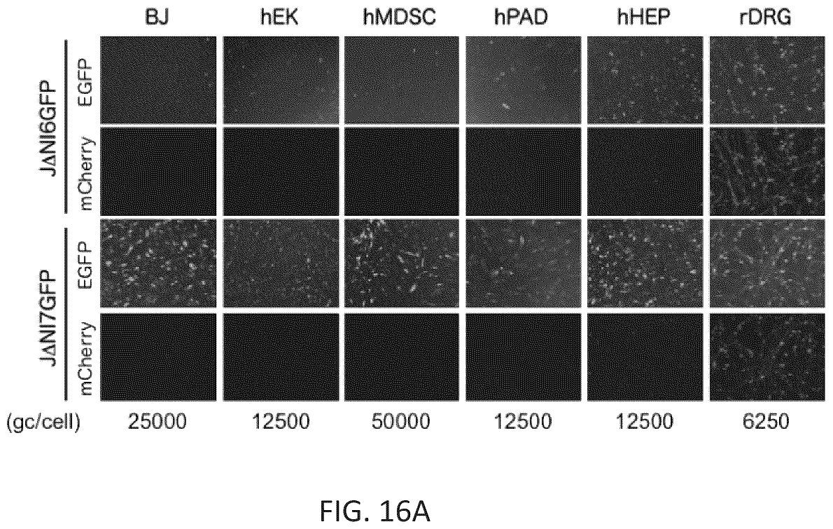

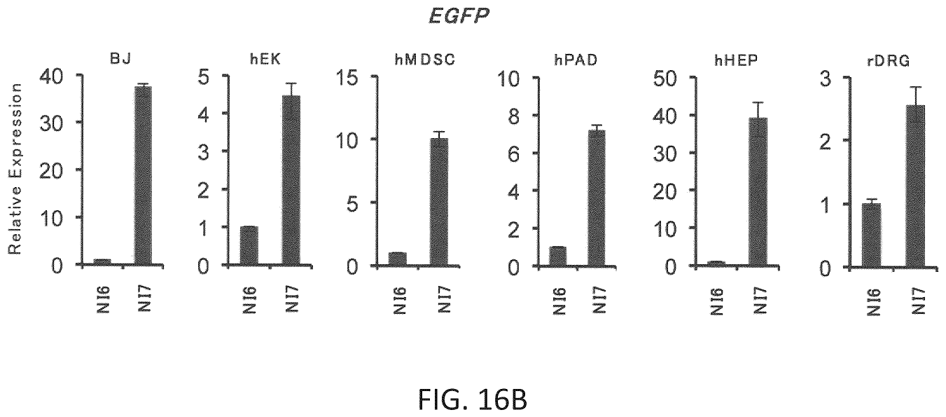

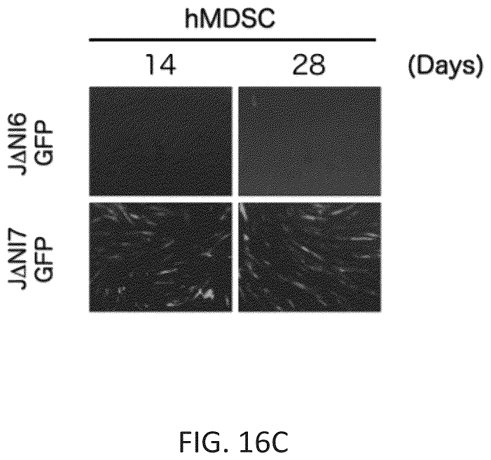

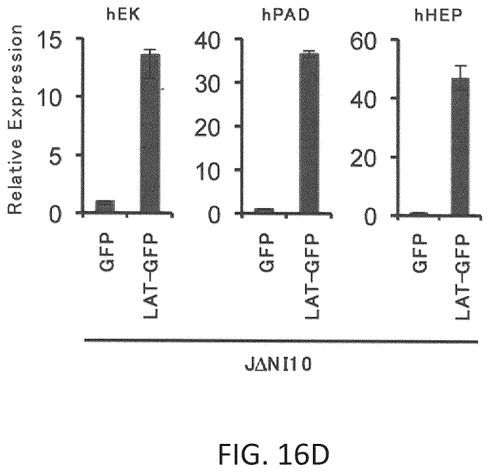

To evaluate the applicability of these findings beyond HDFs, EGFP and mCherry expression from selected vectors was tested in other cell types. By qRT-PCR, higher EGFP mRNA levels were observed in JΔNI7GFP-than in JΔNI6-CAGGFP-infected human cells at 3 dpi (FIG. 16B). The greatest differences were observed in BJ human foreskin fibroblasts (˜35-fold), similar to the difference in HDFs (˜30-fold, FIG. 13C), and in human hepatocytes (hHEP) (˜40-fold). Human neonatal keratinocytes (hEK) showed the smallest difference (˜4.5-fold) with intermediate values seen in muscle-derived stem cells (hMDSC) (˜10-fold) and preadipocytes (hPAD) (˜7-fold). Comparison of the 2 vectors in fetal rat dorsal root ganglion (rDRG) neurons revealed only a ˜2.5-fold difference. FIG. 16A shows representative fluorescence images at 3 dpi from an independent experiment performed under the same infection conditions. With the exception of hHEPs that were from a different donor here than in FIG. 16B, the results were largely consistent with the qRT-PCR data. Of interest, while none of the human cells showed significant mCherry fluorescence, abundant mCherry expression was observed in both the JΔNI6-CAGGFP-and JΔNI7GFP-infected rat DRG cultures. It is not yet known whether this observation is unique to neuronal cells and may be a function of the promoter in front of the mCherry gene, the location of the expression cassette in the viral genome, and/or the rat origin of the cells. hMDSCs were maintained for an extended period of time, allowing the monitoring of EGFP expression over time in JΔNI7GFP-infected cells. As shown in FIG. 16C, EGFP was readily detectable for at least 4 weeks after infection, similar to the observation with JΔNI7GFP-infected HDFs (FIG. 13D).

Lastly, for some of the cells, whether CAGp-GFP activity in the UL50/51 intergenic region was enhanced by flanking LAT sequences as in HDFs was determined. The results in FIG. 16D show a substantial enhancement in all three cell types that may, for unknown reasons, exceed the difference between JΔNI6-CAGGFP and JΔNI7GFP in the same cells. Overall, these results indicated that the position-independent anti-silencing activity of genetic elements in the LAT locus is not limited to HDFs, but is operative in a variety of non-neuronal human cell types.

### Tables for Example 8

TABLE 1
Primer Sequences
PCR primers for qRT-PCR

| SEQ | | | |
|---|---|---|---|
| ID | Target | Forward (F)/ | |
| NO | gene | Reverse (R) | Sequence |
| 2 | Human S18 | F | GCGGCGGAAAATAGCCTTTG |
| 3 | | R | GATCACACGTTCCACCTCATC |
| 4 | EGFP | F | ATCATGGCCGACAAGCAGAAGAAC |
| 5 | | R | GTACAGCTCGTCCATGCCGAGAGT |
| 6 | mCherry | F | CCTGTCCCCTCAGTTCATGT |
| 7 | | R | GCTTCAAGTAGTCGGGGATG |
| 8 | ICP0 | F | ACCACCATGACGACGACTC |
| 9 | | R | AGCCCCGTCTCGAACAGT |
| 10 | ICP22 | F | CGTGTGCAAGCTTCCTTGT |
| 11 | | R | TCTCGAGATTACTAAGATCACACTCC |
| 12 | ICP27 | F | AGTCGTGTCTGCGAGTTGAC |
| 13 | | R | AAAACAGGGAGTTGCAATAAAAA |
| 14 | ICP4 | F | ATGGGGTGGCTCCAGAAC |
| 15 | | R | CTGCCGGTGATGAAGGAG |
| 16 | ICP34.5 | F | GTAACCTCCACGCCCAACT |
| 17 | | R | AGCAGCAGCGAACAAGAAG |
| 18 | UL11 | F | ATGGGCCTCTCGTTCTCC |
| 19 | | R | CGGTGATGAGGACGTTGTT |
| 20 | UL23 (TK) | F | TCATCTTCGACCGCCATC |
| 21 | | R | TGCTGCCCATAAGGTATCG |
| 22 | UL27 (gB) | F | GTCAGCACCTTCATCGACCT |
| 23 | | R | CAGGGGGACAAACTCGTG |
| 24 | UL29 | F | AGCTGCAGATCGAGGACTG |
| | (ICP8) | | |
| 25 | | R | CCATCATCTCCTCGCTTAGG |
| 26 | UL30 | F | GTCTGCTCTACGACCTGTCCA |
| 27 | | R | AACCGAGCGAAAACAGGAG |
| 28 | UL41 (vhs) | F | TCCATCCCAATAACACCTACG |
| 29 | | R | GGGGTCCAGTGACATTCG |
| 30 | UL44 (gC) | F | GAGGGTCAGCCGTTCAAG |
| 31 | | R | AACTCCACGGGGTTACGC |
| 32 | UL48 | F | GCGCTCTCTCGTTTCTTCC |
| | (VP16) | | |
| 33 | | R | GGCCAACACGGTTCGATA |
| 34 | UL39 | F | CAATCTGGGAAGCGTGAATC |
| | (ICP6) | | |
| 35 | | R | CGCCCAAAGTCAAACGTC |
| 36 | Rat S18 | F | CCACAGGAGGCCTACACG |
| 37 | | R | CTGAAACTTCTCGGGGATCA |

Red mediated recombination

| SEQ | Target | | |
|---|---|---|---|
| ID | gene/ | Forward (F)/ | |
| NO | locus | Reverse (R) | |
| 38 | ΔICP0 | F1 | CCCGATATCCAATTGCGGGCGCTGGGTGGTCTCTGGCCGCGCCCACTACACCAGCCAATCCGTG |
| | | | TAGGATGACGACGATAAGTAGGGATA |
| 39 | | F2 | GATCTCGAGCTCAAGCTTCGAATTCTGCAGTCGACGGTACCGCGGGCCCCGATATCCAATTGCG |
| | | | GGC |
| 40 | | R | ACACGGATTGGCTGGTGTAGTGGGCGCGGCCAGAGACCACCCAGCGCCCGCAACCAATTAACC |
| | | | AATTCTGATTAG |
| 41 | ΔICP27 | F | TATGGATCCCGGACCTGGTTAACCACCCGCCGGTCCTACGCGAACTGGAGGATAAGCGCAGGAT |
| | | | GACGACGATAAGTAGGGATA |
| 42 | | R | CAGGAATTCGCGCTTATCCTCCAGTTCGCGTAGGACCGGCGGGTGGTTAACCAGGTCCGCAACC |
| | | | AATTAACCAATTCTGATTAG |
| 43 | Joint | F1 | GGGCCCTGGAAATGGCGGACACCTTCCTGGACACCATGCGGGTTGGGCCCAGGATGACGACGA |
| | deletion | | TAAGTAGGGATAACAGGG |
| 44 | | R | GGGCCCAACCCGCATGGTGTCCAGGAAGGTGTCCGCCATTTCCAGGGCCCCAACCAATTAACCA |
| | | | ATTCTGATTAG |
| 45 | | F2 | TTTATAACCCCGGGGGTCATTCCCAACGATCACATGCAATCTAACTGGCTGGGCCCTGGAAATG |
| | | | GCGGACACC |
| 46 | UbC- | F1 | TATGGATCCCCGCGGATGGTGAGCAAGGGCGAGGAGGATAACATGGCCATCATAGGATGACGA |
| | mCherry | | CGATAAGTAGGG |
| | insertion | | |
| 47 | | R1 | AATCTGCAGGGATCCCTACAACCAATTAACCAATTCTGATTAG |
| 48 | | F2 (For ΔICP4) | CTTGGGGCGGTCCCGCCCGCCGGCCAATGGGGGGGCGGCAAGGCGGGCGGTGGCGGCCGCTCT |
| | | | AGAAGATCTGGC |
| 49 | | R2 (For ΔICP4) | CCGCGGGGGGCCCGGGCTGCCACAGGTGAAACCAACAGAGCACGGCGCACGCTGGGTACCGGG |
| | | | CCCCCCCTCGAG |
| | | F3 (For UL3 and | CCTCACTGCCCGTCGCGCGTGTTTGATGTTAATAAATAACACATAAATTTTGGCGGCCGCTCTAG |
| 50 | | 4) | AAGATCTGGC |
| | | R3 (For UL3 and | CCGACACTGAAATGCCCCCCCCCCCTTGCGGGCGGTCCATTAAAGACAACGCTGGGTACCGGGC |
| 51 | | 4) | CCCCCCTCGAG |
| 52 | CAG-EGFP | F1 | TATTACGTATTAGTCATCGCTATTACCATGGTCGAGGTGAGCCCCACGTTCTGCTTAGGATGACG |
| | insertion | | ACGATAAGTAGGGATA |
| 53 | | R1 | AATCTGCAGTACGTACTACAACCAATTAACCAATTCTGATTAG |
| | | | GCAATTGGCTCTGCCCCGCCGTCCCCGTGTTCGTCC |
| 54 | | F2 | TTTTTGCAAAAGCCTAGGCCTCCAATAACTAGTCAATAATCAATGTCGACTTATTTATTAACATC |
| 55 | | R2 | AAACACGCGC |
| 56 | | F3 | GCACGCGTAGAGGTGCTGCGGGAGATTCAACTGAGC |
| 57 | | R3 | TATTGACTAGTTATTGGAGGCCTAGGCTTTTGCAAAAAAGCTTATAATGGGTCTTTAATGGACCG |
| | | | CCCGCAAGGG |
| 58 | gB:N/T | F | TATTACGTACAACCACATACAGCGCCATGTCAACGATATGTTGGGCCGCGTTGAGGATGACGAC |
| | insertion | | GATAAGTAGGG |
| 59 | | R | AATCTGCAGTACGTACTACAACCAATTAACCAATTCTGATTAG |
| 60 | ΔCTRL1 | F1 | TATACCCGTGACACCCGACGCTGGGGGGCGTGGCTGCCGGGAGGGGCCGCGTATGAGGATGAC |
| | | | GACGATAAGTAGGGATA |
| 61 | | F2 | CCACACAAGCCCCGTATCCCCGTTCCCGCGCTTTTCGTTGGTTTATATACCCGTGACACCCGACG |
| | | | CTGGG |
| 62 | | R | CATACGCGGCCCCTCCCGGCAGCCACGCCCCCCAGCGTCGGGTGTCACGGCAACCAATTAACCA |
| | | | ATTCTGATTAG |
| 63 | ΔCTRL2 | F1 | AATCCAACACAGACAGGGAAAAGATACAAAAGTAAACCTTTATTTCCCAACAAGGATGACGAC |
| | | | GATAAGTAGGGATAACA |
| 64 | | F2 | GTCAGGCAGCCCGGGCCGCGGCTCTGTGGTTAACACCAGAGCCTGCCCAATCCAACACAGACAG |
| | | | GGAAAA |
| 65 | | R | TGTTGGGAAATAAAGGTTTACTTTTGTATCTTTTCCCTGTCTGTGTTGGACAACCAATTAACCAA |
| | | | TTCTGATTAG |
| 66 | ΔLATP2 | F1 | CAGATAGTAATCAATTACGGGGTCATTAGTTCATAGCCCATATATGGAGTTCCAGGATGACGAC |
| | | | GATAAGTAGGGATAAC |
| 67 | | F2 | AGACTTTCCGGGCGCGTCCGGGTGCCGCGGCTCTCCGGGCCCCCCTGCAGATAGTAATCAATTA |
| | | | CGGGGT |
| 68 | | R | GGAACTCCATATATGGGCTATGAACTAATGACCCCGTAATTGATTACTATCAACCAATTAACCA |
| | | | ATTCTGATTAG |
| 69 | GW | F1 (For LAT) | TGACAATTGACTAGTTATACAAGTTTGTACAAAAAAGCTGAAC |
| 70 | | R1 (For LAT) | CCATTATAACAATTGAGATCTCCACTTTGTACAAGAAAGCTGAACG |
| | | F2 (For UL45 and | CGGACCCAAAATAATAAACACACAATCACGTGCGATAAAAAGAACACGCGACAAGTTTGTACA |
| 71 | | 46) | AAAAAGCTGAAC |
| | | R2 (For UL45 and | CCTGTTTGTCGACGAGATTTAATAAAAATAACCAAAAACACCACAGGGGACCACTTTGTACAAG |
| 72 | | 46) | AAAGCTGAACG |
| | | F3 (For UL50 and | AAACGTTTGTATCTCATCTTTCCTGTGTGTAGTTGTTTCTGTTGGATGCCATATCACAAGTTTGTA |
| 73 | | 51) | CAAAAAAGC |
| | | R3 (For UL50 and | TGCGTGTTTTCATCCAACCCGTGTGTTCTGTGTTTGTGGGATGGAGGGGCCGAGAAACGTAAAA |
| 74 | | 51) | TGATATAAATA |
| 75 | ΔLAT | F1 | CCGGGGACACCGCCAGCAAACGCGAGCAACGGGCCACGGGGATGAAGCAGAGGATGACGACG |
| | | | ATAAGTAGGGATA |
| 76 | | F2 | CCACACAAGCCCCGTATCCCCGTTCCCGCGCTTTTCGTTGGTTTATATACCCGGGGACACCGCCA |
| | | | GCAAACGCGA |
| 77 | | R | CTGCTTCATCCCCGTGGCCCGTTGCTCGCGTTTGCTGGCGGTGTCCCCGGCAACCAATTAACCAA |
| | | | TTCTGATTAG |
| 78 | Insertion | F1 (for ICP0 pro) | CCCGGCCAACCAGCGTCCGCCGAGTCTTCGG |
| 79 | of TK | R1 (for ICP0 pro) | GCCGGTTCCAGTGTAAGGGTCGGGGGTCCC |
| 80 | promoter | F2 (for ICP27 pro) | CCTGACAGAGCTGTATTGTA |
| | | R2 (for ICP27 | AGATCGCTGTCGGAGAGGTCC |
| 81 | | pro) | |
| 82 | | F3 (for ICP0 pro) | TATAGATCTCGAGCTCAAGCTTCGAATTCTGCAGTCGACGGTACCGCGGGCCCAGGATGACGAC |
| | | | GATAAGTAGGG |
| 83 | | R3 (for ICP0 pro) | AATCTGCAGAGATCTCTACAACCAATTAACCAATTCTGATTAG |
| 84 | | F4 (for ICP27 | TATGTTAACAACAACAATTGCATTCATTTTATGTTTCAGGTTCAGGGGGAGGTAGGATGACGAC |
| | | pro) | GATAAGTAGGG |
| | | R4 (for ICP27 | AATCTGCAGGTTAACCTACAACCAATTAACCAATTCTGATTAG |
| 85 | | pro) | |

TABLE 2
Viruses used in this example

| Virus | gc/ml | pfu/ml | gc/pfu | ICP0 | ICP4* | ICP22** | ICP27 |
|---|---|---|---|---|---|---|---|
| KOS | 5.33E + 11 | 1.30E + 10 | 4.10E + 01 | + | + | + | + |
| KNTc | 8.15E + 11 | 2.96E + 09 | 2.75E + 02 | + | + | + | + |
| QOZHG | 1.55E + 11 | 2.33E + 08 | 6.64E + 02 | + | − | β | CMV-GFP |
| JΔNI2 | 3.28E + 11 | 4.00E + 08 | 8.20E + 02 | B | − | β | β |
| JΔNI3 | 3.46E + 11 | 3.36E + 08 | 1.03E + 03 | − | − | β | β |
| JΔNI5 | 1.17E + 12 | 5.00E + 08 | 2.35E + 03 | − | − | β | − |
| JΔNI6GFP | 7.30E + 11 | 2.66E + 08 | 2.74E + 03 | − | − | β | − |
| JΔNI7GFP | 3.99E + 11 | 1.86E + 08 | 2.14E + 03 | − | − | β | − |
| JΔNI7GFPΔC1 | 1.66E + 11 | 5.00E + 07 | 3.31E + 03 | − | − | β | − |
| JΔNI7GFPΔC2 | 8.42E + 11 | 3.50E + 08 | 2.41E + 03 | − | − | β | − |
| JΔNI7GFPΔLP2 | 4.75E + 11 | 1.70E + 08 | 2.80E + 03 | − | − | β | − |
| JΔNI7GFPΔC12 | 5.97E + 11 | 1.86E + 08 | 3.21E + 03 | − | − | β | − |
| JΔNI7GFPΔC | 8.03E + 11 | 3.00E + 08 | 2.68E + 03 | − | − | β | − |
| 12LP2 | | | | | | | |
| JΔNI9GW | − | − | − | − | − | β | − |
| JΔNI9GFP | 6.70E + 11 | 2.00E + 08 | 3.35E + 03 | − | − | β | − |
| JΔNI9LAT-GFP | 5.58E + 11 | 2.66E + 08 | 2.10E + 03 | − | − | β | − |
| JΔNI10GW | − | − | − | − | − | β | − |
| JΔNI10GFP | 1.05E + 12 | 1.66E + 08 | 6.33E + 03 | − | − | β | − |
| JΔNI10LAT-GFP | 2.30E + 11 | 9.33E + 07 | 2.46E + 03 | − | − | β | − |
| JΔNI10LAT- | 2.69E + 11 | 1.23E + 08 | 2.19E + 03 | − | − | β | − |
| GFPΔC12LP2 | | | | | | | |
| | Virus | ICP47 | gB | LAT | UL₃₋₄ | UL₅₀₋₅₁ | UL₄₅₋₄₆ |
| | KOS | + | + | + | − | − | − |
| | KNTc | + | N/T*** | + | UbC-mCherry | − | − |
| | QOZHG | β | + | + | − | − | − |
| | JΔNI2 | − | N/T | + | − | − | − |
| | JΔNI3 | − | N/T | + | − | − | − |
| | JΔNI5 | − | N/T | + | − | − | − |
| | JΔNI6GFP | − | N/T | + | CAG-GFP | − | − |
| | JΔNI7GFP | − | N/T | CAG-GFP | − | − | − |
| | JΔNI7GFPΔC1 | − | N/T | CAG-GFP, | − | − | − |
| | | | | ΔCTRL1 | | | |
| | JΔNI7GFPΔC2 | − | N/T | CAG-GFP, | − | − | − |
| | | | | ΔCTRL2 | | | |
| | JΔNI7GFPΔLP2 | − | N/T | CAG-GFP, | − | − | − |
| | | | | ΔLATP2 | | | |
| | JΔNI7GFPΔC12 | − | N/T | CAG-GFP, | − | − | − |
| | | | | ΔCTRL1/2 | | | |
| | JΔNI7GFPΔC | − | N/T | CAG-GFP, | − | − | − |
| | 12LP2 | | | ΔCTRL1/2/ | | | |
| | | | | LATP2 | | | |
| | JΔNI9GW | − | N/T | − | − | − | GW**** |
| | JΔNI9GFP | − | N/T | − | − | − | CAG-GFP |
| | JΔNI9LAT-GFP | − | N/T | − | − | − | LAT-CAG-GFP |
| | JΔNI10GW | − | N/T | − | − | GW**** | − |
| | JΔNI10GFP | − | N/T | − | − | CAG-GFP | − |
| | JΔNI10LAT-GFP | − | N/T | − | − | LAT-CAG-GFP | − |
| | JΔNI10LAT- | − | N/T | − | − | LAT-ΔCTRL1/2/ | − |
| | GFPΔC12LP2 | | | | | LATP2-CAG-GFP | |

*All BAC-mediated ΔICP4 viruses have the same UbC promoter-driving mCherry expression cassette in the deleted ICP4 locus
**For all BAC-mediated ICP22 viruses, the regulatory sequences of ICP22 promoter (TAATGARAT) were deleted to convert the kinetics of ICP22 gene expression from IE to early gene
***gB:D285N/A549T, referred to herein as gB:N/T
****GW: Gateway recombination site

TABLE 3
JΔNI5 plaquing efficiency on U2OS-ICP4/27
cells at different passage numbers

| | |
|---|---|
| Passage number | Plaque number (mean ± S.D.) |

| | | |
|---|---|---|
| p5 | 17.33 | ±6.35 |
| p10 | 15 | ±2.64 |
| p20 | 16 | ±4 |

U2OS-ICP4/27 cells were infected with JΔNI5 virus at 0.1 gc/cell and the cells were overlayed with methylcellulose at 2 hpi.
Plaques were counted at 4 dpi.
Means +/− SD of 3 determinations.

### EXAMPLE 9

This example demonstrates the construction and properties of JΔNI8.

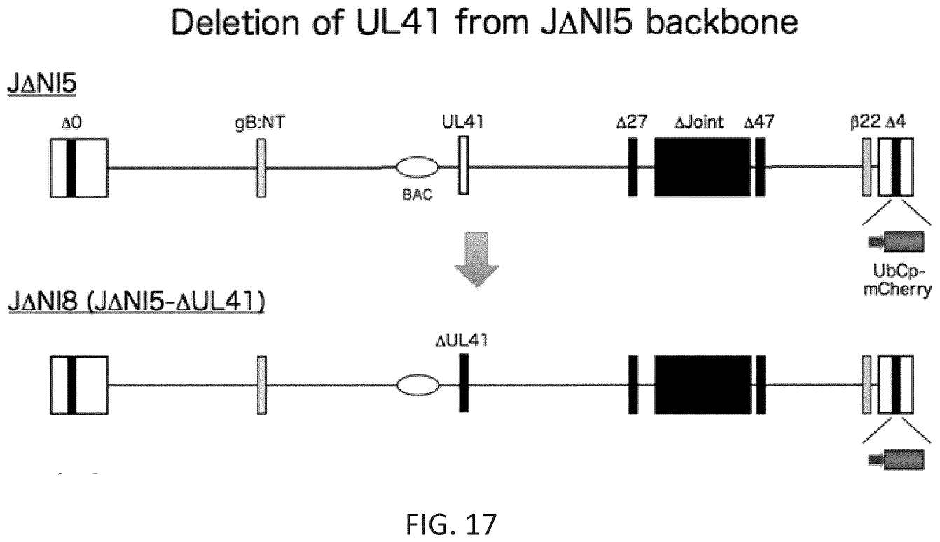

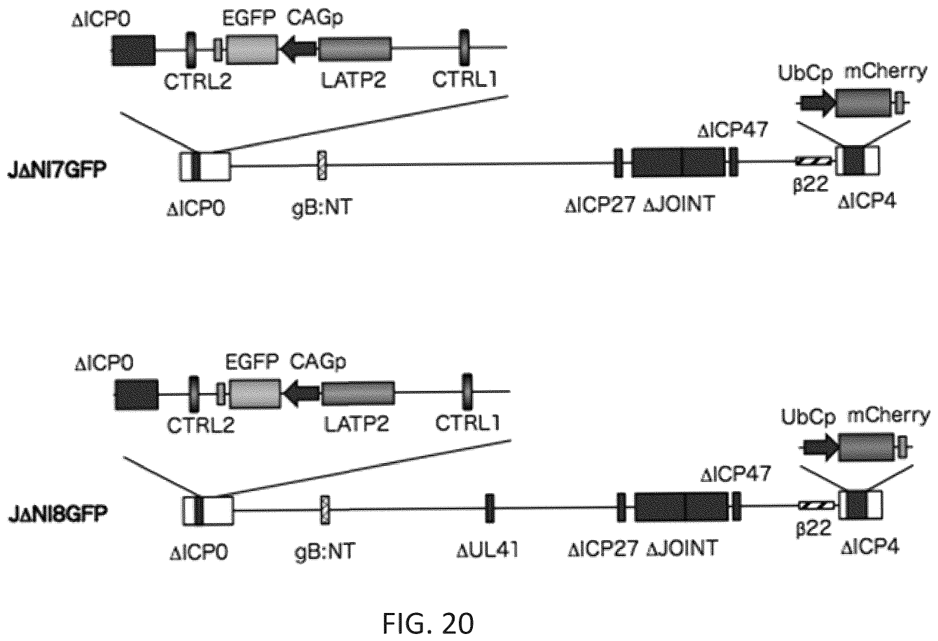

JΔNI8 was constructed by deletion of UL41 from the JΔNI5vector discussed above. This is presented graphically in FIG. 17. JΔNI7GFP essentially is JΔNI8GFP is identical to JΔNI7GFP except for the deletion of UL41. See also FIG. 20.

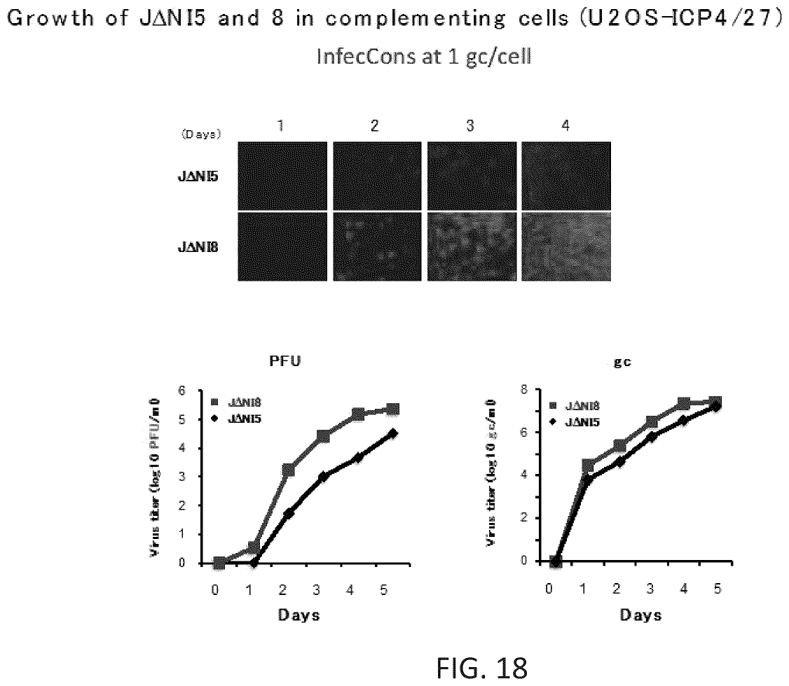

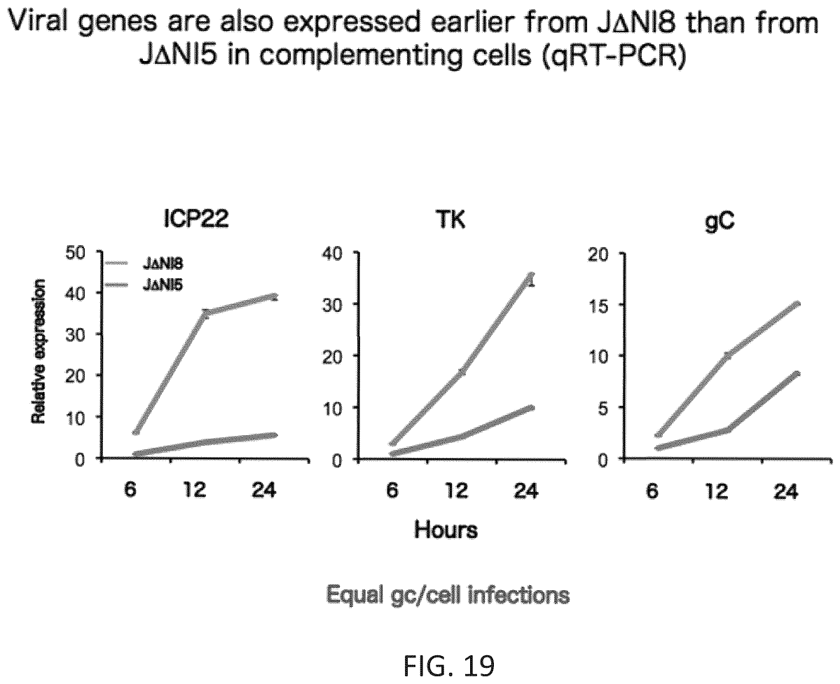

The growth of JΔNI8 was assessed relative to JΔNI5 in complementing cells, which, as described above, are U2OS cells engineered to express ICP4 and ICP27. The complementing cells were infected with the respective vector at 1 viral genome copy (gc) per cell. As depicted in FIG. 18, as measured by plaque forming units (PFU), genomic copy (gc) and expression of the mCherry transgene, JΔNI8 exhibited more robust growth than JΔNI5. Furthermore, native HSV genes were observed to be expressed earlier during infection in complementing cells from JΔNI8 as opposed to JΔNI5(FIG. 19, representing fold expression relative to each JDNI5 gene at 6 hpi arbitrarily assigned a value of 1).

As revealed in Table 9-1, JΔNI8 and JΔNI8GFP produced more plaques per genomic copy (gc) (on complementing cells) than JΔNI5 and JΔNI7GFP, respectively. This reveals that the ratio of functional over defective particles (a measure of quality) in JΔNI8 and JΔNI8GFP preparations is higher than in JΔNI5 and JΔNI7 GFP preparations.

TABLE 9-1

| Virus | gc/ml | pfu/ml | gc/pfu |
|---|---|---|---|
| JΔNI5 #1 | 1.98E+11 | 8.33E+07 | 2.38E+03 |
| JΔNI5 #2 | 1.17E+12 | 5.00E+08 | 2.35E+03 |
| JΔNI7GFP | 3.99E+11 | 1.86E+08 | 2.14E+03 |
| JΔNI8 #1 | 3.72E+10 | 4.66E+07 | 7.99E+02 |
| JΔNI8 #2 | 5.56E+11 | 1.06E+09 | 5.24E+02 |
| JΔNI8GFP | 5.33E+11 | 8.33E+08 | 6.40E+02 |

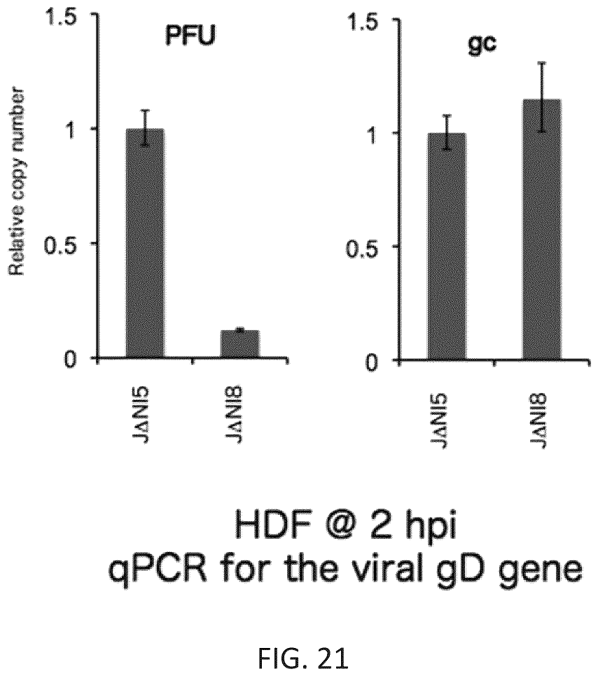

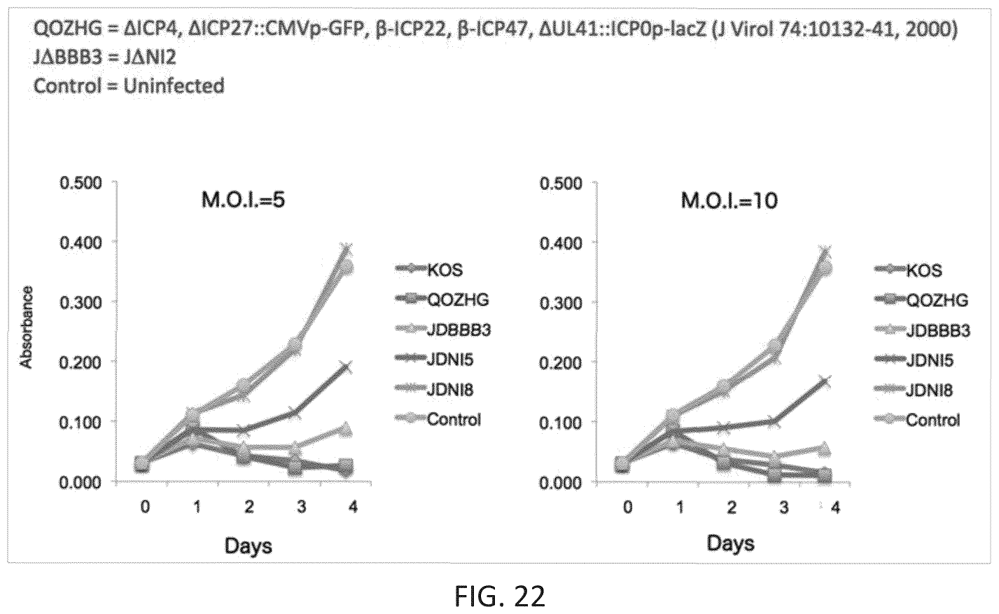

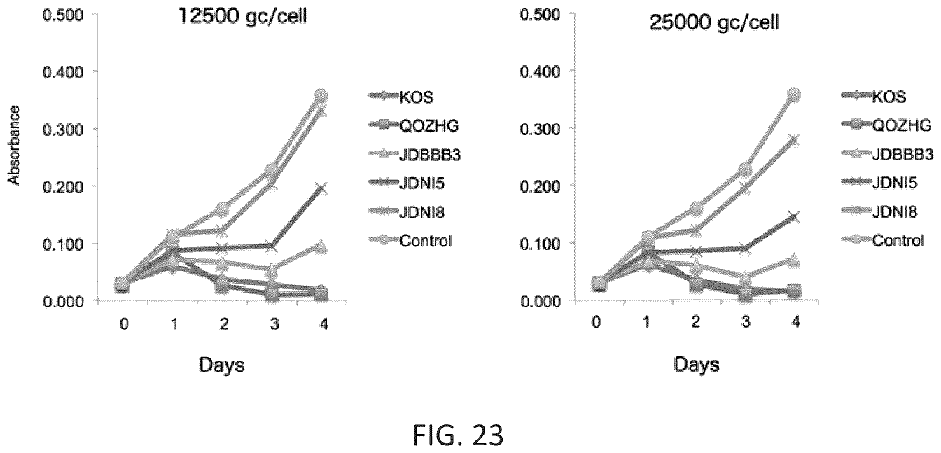

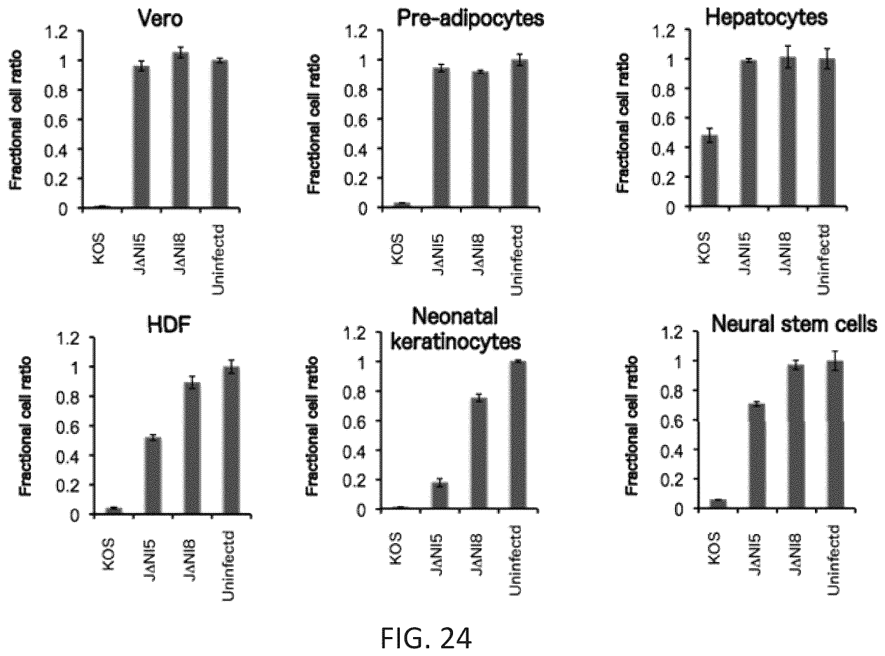

Also, it was observed that infection at equal levels of gc resulted in equal amounts of viral DNA in the nucleus of human dermal fibroblasts. However, using the protocol discussed in connection with FIG. 10, this was accomplished by much lower PFU for JΔNI8 vs. JΔNI5. (FIG. 21) These data reveal that infections at equal gc should be used for studies comparing JΔNI8 to JΔNI5 (or JΔNI7GFP vs JΔNI8GFP). MTT cell viability analysis (see FIG. 22) demonstrated that JΔNI8 (upper-X-line in each graph) was measurably less toxic (approaching or exceeding the viability of uninfected control cells) than any of the other vectors at 5 or 10 PFU/cell (M.O.I.=5 or =10). The lower-X-line in each graph represents the results for JΔNI5. These data also reveal that vectors that have an intact ICP0 gene, even under a β promoter (JΔBBB3 (also termed JDBBB3 or JDΔβββ3 or JDBβββ3)) or without UL41 (QOZHG), are more toxic than JΔNI5 or JΔNI8. As expected, replicating virus (KOS) kills the cells. Additional MTT experiments using HDFs reveal that, although JΔNI8 showed slight toxicity at higher MOIs it was much less than JΔNI5 at 3-fold lower MOIs (equal gc/cell) (FIG. 23). Other MTT data revealed that JΔNI8 is less toxic than JΔNI5 in some (HDF, human neonatal keratinocytes, and human neural stem cells) but not all (Vero, human preadipocytes, and human hepatocytes) cell types (see FIG. 24).

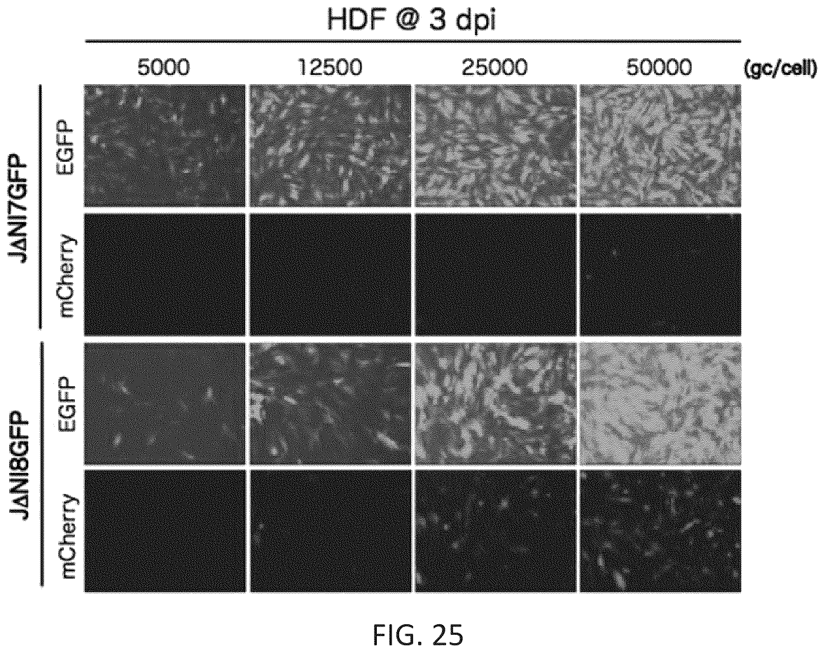

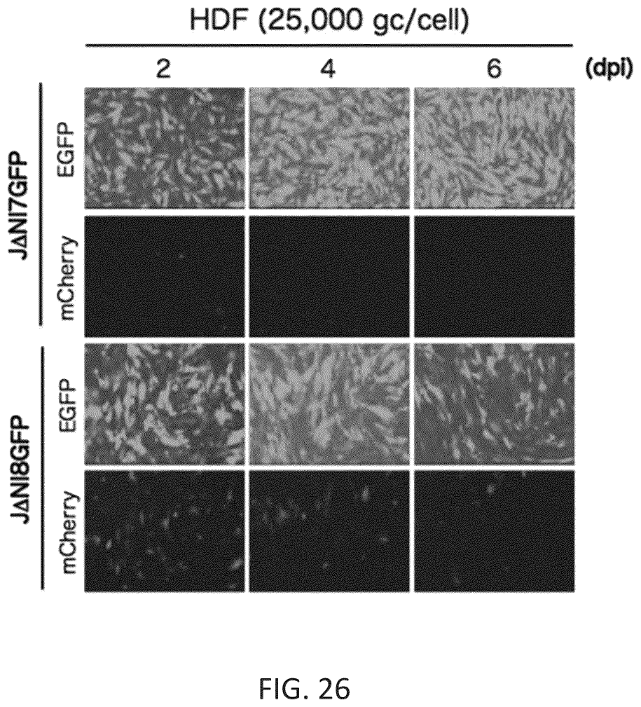

Reporter gene expression from both transgenes (i.e., EGFP and mCherry) present within JΔNI7GFP and JΔNI88GFP, respectively, were compared in HDF cells at 3 dpi. The results are presented in FIGS. 25 and 26. It was observed that GFP expression by JΔNI8GFP increased with dose (from 5000 to 50,000 gc/cell, FIG. 25), but leveled off for JΔNI7GFP. Without wishing to be bound by theory, it is believed that the leveling-off observed for expression from JΔNI7GFP may be attributed to high-dose toxicity of the vector in HDF cells, which implies that such toxity is lower in JΔNI8GFP. Also, notable mCherry was observed from the HDF cells infected with JΔNI8GFP but not by HDF cells infected with JΔNI7GFP. Similar experiments were conducted with a constant dose of either JΔNI7GFP or JΔNI8GFP (25,000 gc/cell) with the level of EGFP or mCherry fluorescence assayed over time (2, 4, and 6 days post infection (FIG. 26)). Reporter signals in JΔNI8GFP-infected cultures decreased between 4 and 6 dpi, likely a result of viral genome dilution by unimpaired cell division. Signals were more stable in JΔNI7GFP-infected cultures, likely due to reduced cell division.

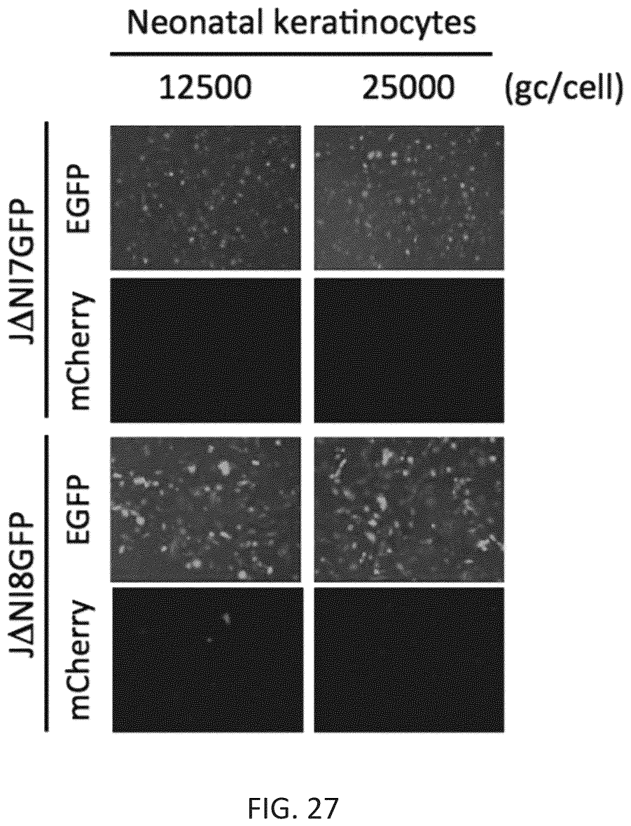

Transgene (EGFP or mCherry) expression from other cells infected with either JΔNI7GFP vs. JΔNI8GFP also was investigated. As depicted in FIG. 27, at two measured doses (12,500 gc/cell and 25,000 gc/cell) JΔNI8GFP expressed more GFP in human neonatal keratinocytes than JΔNI77GFP.

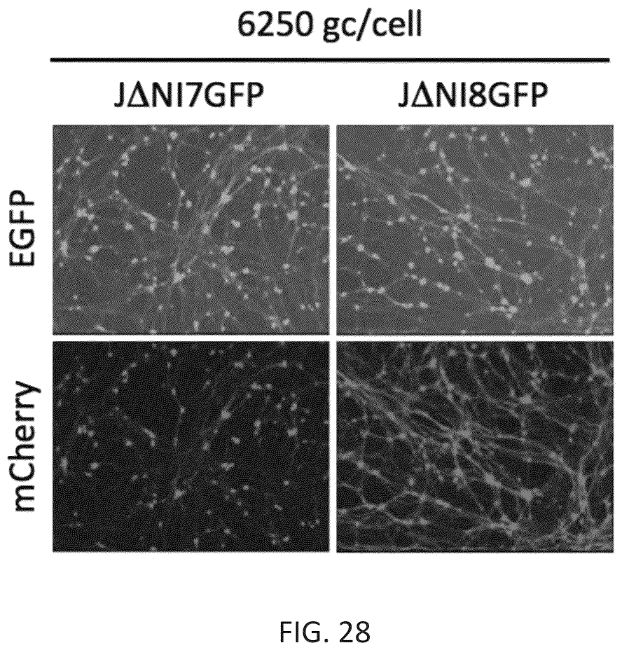

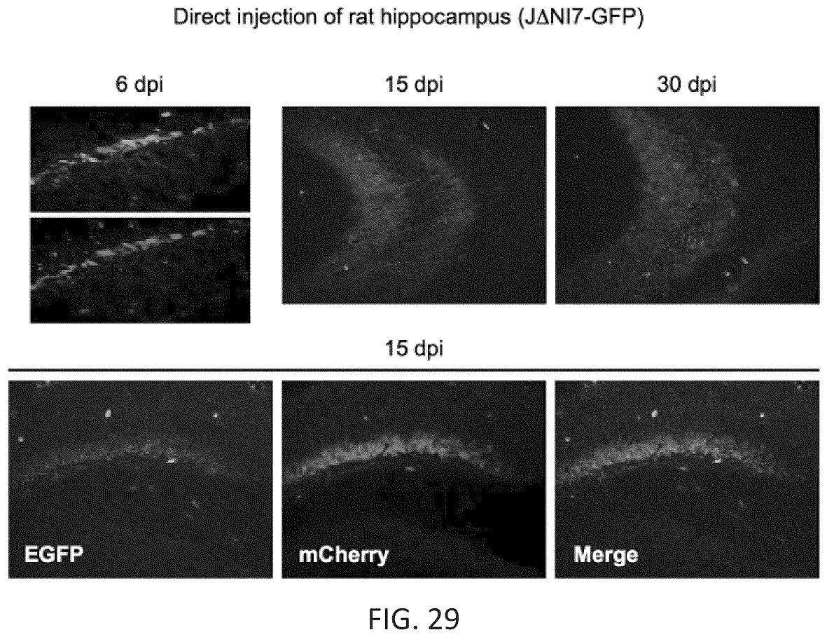

Also, transgene expression from rat dorsal root ganglion (DRG) cells infected with either either JΔNI7GFP or JΔNI8GFP (at 6250 gc/cell) was assayed. As depicted in FIG. 28, mCherry (inserted in the ICP4 deleted locus of both vectors) expression from JΔNI7GFP was enhanced in rat DRG neurons relative to GFP compared to non-neuronal cells, and relatively more in JΔNI8GFP—than in JΔNI7GFP-infected cells. Also, as depicted in FIG. 29 expression of both EGFP and mCherry was observed in rat hippocampus at at least 15 days following direct injection of the hippocampus with JΔNI7GFP These results reveal that JΔNI8 is non toxic and provides for transgene expression in neurons from both the LAT and the ICP4 locus.

### EXAMPLE 10

This example demonstrates in vivo expression from the JΔNI7-GFP vector in a long-term experiment.

JDNI7-GFP (8.0×10⁸ genome copies/2 ml; 3.7×10⁵ PFU) was sterotactically injected into the hippocampus of rats (AP: −1.8; ML: −1.7; P: +3.5) at a rate of 0.1 ul/min. Animals were euthanized and perfused at 6, 15 or 30 days post vector injection (dpi), and brain cryosections were imaged under a fluorescence microscope. FIG. 29 reveals robust expression of both EGFP and mCherry co-localized within the same cells within the hippocampus. In FIG. 29, separate (left: 40×) or merged (center, right: 20×) images of EGFP and mCherry fluorescence at the indicated days post infection (dpi); bottom, separate and merged images are depicted at 15 dpi (10×).

### EXAMPLE 11

This example demonstrates the expression of the mCherry transgene from the JΔNI7-GFP vector within infected primary astrocytes.

Mouse astrocytes were infected (M.O.I.=5) and cultured. After 25 days post infection, the cells were fixed and exposed to an antibody specific to glial fibrillary acidic protein (GFAP), positive binding of which identifies astrocytes. Immunofluorescence imaging reveals that JΔNI7-GFP is non toxic and provides for persistent expression in astrocytes.

### EXAMPLE 12

This example demonstrates the generation of a cell line for complementing the inventive HSV vectors and which also facilitates exision of a loxP-flanked BAC cassette during viral propagation.

The U2OS-ICP4/27 cell line discussed above was engineered to express Cre recombinase. The resulting cell line, U2OS-ICP4/27/Cre, complements the functions of all of the deficient HSV IE genes in vectors JDNI5, 7, 8 and their derivatives except that of the dispensable ICP47 gene, and removes the BAC cassette during virus growth.

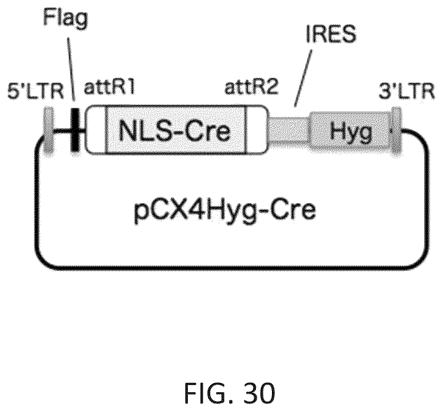

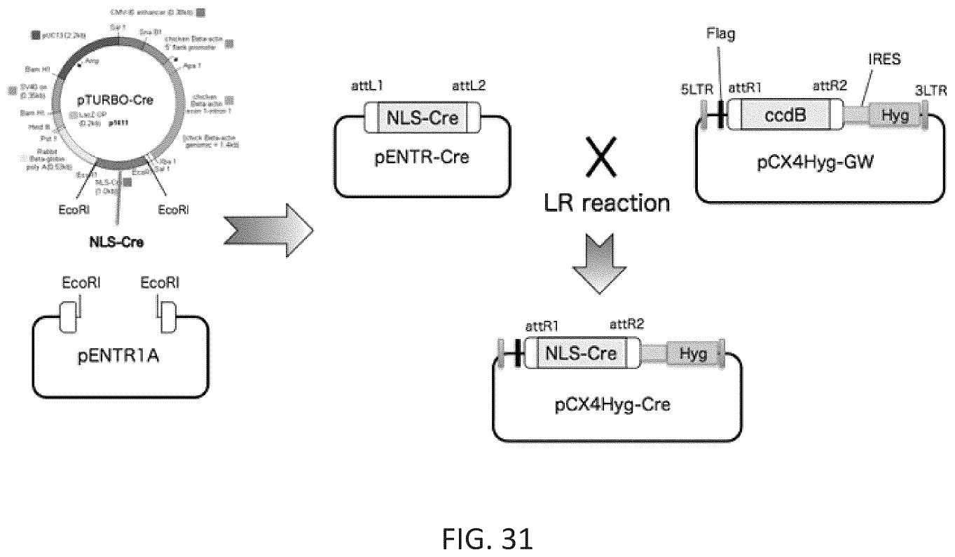

U2OS-ICP4/27/Cre cells were generated by infection of U2OS-ICP4/27 cells with a Cre-expressing retroviral vector. For construction of the retroviral vector, plasmid pCX4Hyg (PNAS 2003, 100:13567-13572; GenBank accession number AB086387) was modified by insertion of a Gateway recombination cassette into the multi cloning site to create pCX4Hyg-GW. The complete NLS-Cre coding sequence from plasmid pTurbo-Cre (GenBank accession number AF334827.1) was inserted between the attL1 and attL2 sites of pENTRIA (Invitrogen) and then transferred into pCX4Hyg-GW by LR Clonase-mediated Gateway recombination to create pCX4Hyg-Cre. This plasmid and its construction are schematically depicte in FIGS. 30 and 31. Retroviral particles were produced as described by Makino et al. (Exp Cell Res 2009, 315:2727-2740). Briefly, pCX4Hyg-Cre was co-transfected with Gag-Pol, and VSV-G expression plasmids into 293T cells and supernatant was collected 48 hours later, filtered through a 0.45 micron filter, and concentrated by centrifugation. Suitable Gag-Pol & VSV-G plasmids are commercially available (e.g., pCMV-Gag-Pol from Cell Biolabs (Cat. No. RV-111) and pCMV-VSV-G from Addgene).

U2OS-ICP4/27 cells were infected with the purified Cre retrovirus and cells were selected for resistance to puromycin, blasticidin and hygromycin. Resistant colonies were isolated, amplified, infected with JΔNI7-GFP at 0.001-1 PFU/cell, and stained for β-galactosidase activity at 2 days post infection (dpi). Clones showing few blue cells were found to undergo rapid cytopathic effect (100% CPE at 4 dpi after infection at MOI=0.01) whereas the parental U2OS-ICP4/27 cells showed plaque-forming clusters of blue cells and approximately 25% CPE at 9 dpi. These results indicated that Cre-mediated removal of the BAC elements along with the linked LacZ expression cassette in JΔNI7-GFP facilitated virus growth. Accurate removal of the BAC and LacZ sequences between the loxP sites of JΔNI8 -GFP has been confirmed by PCR on viral DNA from individual JΔNI7-GFP plaques on a selected U2OS-ICP4⁺/27⁺/Cre*⁺ clone.

It thus has been observed that passage through this U2OS-ICP4/27/Cre cell line removes the approximately 11KB BAC sequence, recovering room for transgenes, and markedly accelerates virus growth.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having.” “including.” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A method of expressing a transgene in a human dermal cell of a subject in need thereof, comprising administering to the subject a herpes simplex virus (HSV) vector, wherein said transgene is inserted into a latency-associated transcript (LAT) region of said HSV vector, and wherein said HSV vector comprises an inactivating mutation within an ICP0 gene, an inactivating mutation within an ICP4 gene, and an inactivating mutation within an ICP27 gene.

3. The method of claim 2, wherein said HSV vector comprises an inactivating mutation within an UL41 gene.

4. The method of claim 2, wherein said HSV vector comprises an inactivating mutation within an ICP22 gene.

5. The method of claim 2, wherein said HSV vector does not detectably express the ICP22 gene as an immediate early gene.

6. The method of claim 2, wherein said HSV vector comprises an inactivating mutation within an ICP47 gene.

7. The method of claim 2, wherein said HSV vector comprises a deletion within a joint region.

8. The method of claim 2, wherein said transgene is expressed for at least 28 days.

9. The method of claim 8, wherein said transgene is expressed for at least 60 days.

10. The method of claim 2, wherein said LAT region comprises an insulator sequence, an enhancer element, or a combination thereof.

11. The method of claim 10, wherein said transgene is operably linked to said insulator sequence, said enhancer element, or a combination thereof.

12. The method of claim 10, wherein said insulator sequence is a CTRL1 sequence, a CTRL2 sequence, or a combination thereof, and wherein said enhancer element is a LATP2 enhancer element, a LAP2 enhancer element, or a combination thereof.

13. The method of claim 12, wherein said transgene is inserted between said CTRL1 sequence and said CTRL2 sequence.

14. The method of claim 12, wherein said transgene is inserted between the said LATP2 enhancer element and said CTRL1 sequence.

15. The method of claim 12, wherein said transgene is inserted between said LATP2 enhancer element and said CTRL2 sequence.

16. The method of claim 2, wherein said transgene is operably linked to a constitutive promoter.

17. The method of claim 16, wherein said promoter is a constitutive mammalian promoter.

18. The method of claim 2, wherein said transgene encodes Oct4, Klf4, Sox2, c-Myc, L-myc, dominant-negative p53, Nanog, Glis1, Lin28, TFIID, GATA4, Nkx2.5, Tbx5, Mef2C, Myocd, Hand2, SRF, Mesp1, SMARCD3, SERCA2a, Pax3, MyoD, Lhx2, FoxG1, FoxP2, Is1l, Ctip2, Tbr1, Ebf1, Gsx2, Srebp2, Factor VIII, Factor IX, Dystrophin, CFTR, GlyRa1, enkephalin, a GAD isoform, a neurotrophic factor, Ascii, Nurr1, Lmx1A, Brn2, Myt1l, NeuroD1, FoxA2, Hnf4a, Foxa1, Foxa2 or Foxa3, a microRNA, combination of miRNAs, or one or more non-coding RNA(s) (ncRNA(s)).

19. The method of claim 2, wherein said human dermal cell is a non-complementing cell.

20. The method of claim 2, wherein said human dermal cell is a fibroblast.

21. The method of claim 2, wherein said HSV vector comprises a bacterial artificial chromosome (BAC).

22. A method of expressing a transgene in a human dermal cell, comprising infecting said human dermal cell with a herpes simplex virus (HSV) vector, wherein said transgene is inserted into a latency-associated transcript (LAT) region of said HSV vector, and wherein said HSV vector comprises an inactivating mutation within an ICP0 gene, an inactivating mutation within an ICP4 gene, and an inactivating mutation within an ICP27 gene.
